(19) United States
(12) Patent Application Publication
(10) Pub. No.: US 2025/0011396 A1
(43) Pub. Date: Jan. 9, 2025

(54) MULTIVALENT CARRIERS AND RELATED VACCINE COMPOSITIONS

(71) Applicant: California Institute of Technology, Pasadena, CA (US)

(72) Inventors: Alexander A. Cohen, Pasadena, CA (US); Pamela J. Bjorkman, Altadena, CA (US)

(21) Appl. No.: 18/811,229

(22) Filed: Aug. 21, 2024

Related U.S. Application Data
(63) Continuation-in-part of application No. 17/523,813, filed on Nov. 10, 2021.
(60) Provisional application No. 63/112,495, filed on Nov. 11, 2020.

(51) Int. Cl. C07K 16/10; A61K 39/00; A61K 39/215; A61P 37/04

(52) U.S. Cl. CPC C07K 16/1002; A61K 39/215; A61P 37/04; A61K 2039/70; C07K 2317/33

(57) ABSTRACT

Disclosed herein include multivalent carriers comprising a plurality of heterologous coronavirus proteins antigens derived from different coronaviruses. The multivalent carriers herein described can elicit heterologous binding and neutralization properties against coronaviruses that differ from the coronaviruses from which the coronavirus antigens are derived to produce the multivalent carriers. Also provided herein include vaccine compositions comprising the multivalent carriers and related methods using the vaccine compositions in various therapeutic and prophylactic applications.

### RELATED APPLICATIONS

The present application is a continuation-in-part of U.S. application Ser. No. 17/523,813, filed Nov. 10, 2021, which claims priority to U.S. Provisional Application No. 63/112,495, filed Nov. 11, 2020. The content of this related application is hereby expressly incorporated by reference in its entirety.

### STATEMENT REGARDING FEDERALLY SPONSORED R&D

This invention was made with government support under Grant No(s). AI138938, AI100148 awarded by the National Institutes of Health. The government has certain rights in the invention.

### REFERENCE TO SEQUENCE LISTING

The present application is being filed along with a Sequence Listing in electronic format. The Sequence Listing is provided as a file entitled Sequence_Listing_30KJ-302430-US2, created Aug. 13, 2024, which is 28,338 bytes in size. The information in the electronic format of the Sequence Listing is incorporated herein by reference in its entirety.

### BACKGROUND

### Field

The present disclosure relates generally to the field of immune medicine, in particular, to vaccines against viral disorders.

### Description of the Related Art

Severe acute respiratory syndrome (SARS) is a severe emerging viral disease caused by a coronavirus infection with high fatality characterized by fever, headache and severe respiratory symptoms including cough, dyspnea and pneumonia. Two zoonotic Betacoronaviruses, SARS-CoV and MERS-CoV, resulted in outbreaks within the last 20 years. A newly-emergent Betacoronavirus, SARS-CoV-2, resulted in a global pandemic in 2020, infecting millions and causing the respiratory disease COVID-19. Protection against SARS-CoV-2 and SARS-related emergent zoonotic coronaviruses is urgently needed.

### SUMMARY

Provided herein include multivalent carriers and related vaccine compositions, methods for using the multivalent carriers and the vaccine compositions, and kits for protecting a subject against coronavirus infections.

Disclosed herein include a vaccine composition. The vaccine composition can, for example, comprise a multivalent carrier associated with a plurality of coronavirus antigens, wherein the plurality of coronavirus antigens comprises a first coronavirus antigen of a first coronavirus and a second coronavirus antigen of a second coronavirus that is different from the first coronavirus. The plurality of coronavirus antigens can be displayed on the surface of the multivalent carrier, or partially (e.g., substantially) embedded in the multivalent carrier. The plurality of coronavirus antigens can comprise a coronavirus spike protein (S protein) or a portion thereof, a coronavirus nucleocapsid protein (N protein) or a portion thereof, a coronavirus hemagglutinin-esterase protein (HE protein) or a portion thereof, a coronavirus papain-like protease or a portion thereof, a coronavirus 3CL protease or a portion thereof, a coronavirus membrane protein (M protein) or a portion thereof, or a combination thereof.

The plurality of coronavirus antigens can comprise coronavirus proteins of a same protein type. In some embodiments, the plurality of coronavirus antigens comprise coronavirus proteins of different protein types. The plurality of coronavirus antigens can, for example, comprise a coronavirus S protein or a portion thereof. In some embodiments, the plurality of coronavirus antigens comprise a coronavirus S protein receptor binding domain (RBD) or a portion thereof. In some embodiments, the first coronavirus antigen, the second coronavirus antigen, or both comprise a coronavirus S protein or a portion thereof. In some embodiments, the first coronavirus antigen, the second coronavirus antigen, or both comprise a coronavirus S protein RBD or a portion thereof. One or more of the plurality of coronavirus antigens, or each of the plurality of coronavirus antigens, can have a sequence identity of, of about, of at least, or of at least about, 65%, 70%, 75%, 80%, 85%, 90%, 95%, 98%, or 99% with one another. In some embodiments, the plurality of coronavirus antigens each comprises a coronavirus S protein RBD or a portion thereof, the coronavirus S protein RBDs or portions thereof having a sequence identity of about, at least, or at least about 65%, 70%, 75%, 80%, 85%, 90%, 95%, 98%, or 99% with one another. In some embodiments, one or more of the plurality of coronavirus antigens, or each of the plurality of coronavirus antigens, comprise (1) an amino acid sequence having at least 65%, 70%, 75%, 80%, 85%, 90%, 95%, 98%, or 99% sequence identity to an amino acid sequence selected from SEQ ID NOs: 1-12 and 15-26; or (2) an amino acid sequence selected from SEQ ID NOs: 1-12 and 15-26. For example, the first coronavirus antigen can comprise (1) an amino acid sequence having at least 65%, 70%, 75%, 80%, 85%, 90%, 95%, 98%, or 99% sequence identity to an amino acid sequence selected from SEQ ID NOs: 1-12 and 15-26; or (2) an amino acid sequence selected from SEQ ID NOs: 1-12 and 15-26. The second coronavirus antigen can comprise, for example, (1) an amino acid sequence having at least 65%, 70%, 75%, 80%, 85%, 90%, 95%, 98%, or 99% sequence identity to an amino acid sequence selected from SEQ ID NOs: 1-12 and 15-26; or (2) an amino acid sequence selected from SEQ ID NOs: 1-12 and 15-26. The number of the first coronavirus antigen molecules and the number of the second coronavirus antigen molecules can be, for example, in a ratio from 1:100 to 100:1.

In some embodiments, the plurality of coronavirus antigens comprise three, four, five, size seven, or eight coronavirus antigens, each of a coronavirus different from one another. In some embodiments, the plurality of coronavirus antigens further comprise at least a third coronavirus antigen of a third coronavirus and a fourth coronavirus antigen of a fourth coronavirus, and wherein the first, second, third and fourth coronaviruses are different from one another. In some embodiments, the plurality of coronavirus antigens further comprise one or more of a third coronavirus antigen of a third coronavirus, a fourth antigen coronavirus of a fourth coronavirus, a fifth coronavirus antigen of a fifth coronavirus, a sixth coronavirus antigen of a sixth coronavirus, a seventh coronavirus antigen of a seventh coronavirus, and an eighth coronavirus antigen of an eighth coronavirus, and wherein the first, second, third, fourth, fifth, sixth, seventh and eighth coronaviruses are different from one another. In some embodiments, the vaccine composition comprises three, four, five, six, seven, or eight coronavirus S protein RBDs, and wherein each of the plurality of coronavirus S protein RBDs is different from one another.

One or more of the plurality of coronavirus antigens can be of coronaviruses in the genus of Alpha-coronavirus and/or Beta-coronavirus. In some embodiments, each of the plurality of coronavirus antigens are of coronaviruses in the genus of Beta-coronavirus. In some embodiments, the plurality of coronavirus antigens are of coronaviruses in the subgenus of Sarbecovirus. In some embodiments, the first coronavirus and the second coronavirus are in the genus of Beta-coronavirus, optionally in the subgenus of Sarbecovirus. The plurality of coronavirus antigens can be of coronaviruses selected from: SARS, SARS-2, WIV1, SHC014, Rf1, RmYN02, pang17, RaTG13, and Rs4081. For example, the first coronavirus, the second coronavirus, or both can be selected from: SARS, SARS-2, WIV1, SHC014, Rf1, RmYN02, pang17, RaTG13, and Rs4081.

The plurality of coronavirus antigens can be covalently attached to the multivalent carrier, and optionally the plurality of coronavirus antigens are conjugated to the multivalent carrier. In some embodiments, the plurality of coronavirus antigens are attached to the multivalent carrier through click chemistry. In some embodiments, the plurality of coronavirus antigens are non-covalently attached to the multivalent carrier. Non-limiting examples of the multivalent carrier include nanoparticles, nanotubes, nanowires, dendrimers, liposomes, ethosomes and aquasomes, polymersomes and niosomes, foams, hydrogels, cubosomes, quantum dots, exosomes, macrophages, and combinations thereof. In some embodiments, the multivalent carrier comprises a nanoparticle selected from: lipid-based nanoparticles, polymeric nanoparticles, inorganic nanoparticles, surfactant-based emulsions, nanowires, silica nanoparticles, virus-like particles, peptide or protein-based particles, lipid-polymer particles, nanolipoprotein particles, and combinations thereof. The multivalent carrier can, for example comprise a virus-like particle (VLP), for example the virus-like particle can be Ap205 VLP. In some embodiments, the multivalent carrier comprises a self-assembling nanoparticle. The self-assembling nanoparticle can be, for example, an i301 nanoparticle or a variant thereof, or an a mi3 nanoparticle or a variant thereof.

In some embodiments, the vaccine composition comprises a plurality of particle-forming proteins. One or more of the plurality of particle-forming proteins can, for example, comprise a 2-dehydro-3-deoxy-phosphogluconate (KDPG) aldolase or a variant thereof. In some embodiments, a coronavirus antigen of the plurality of coronavirus antigens is attached to a particle-forming protein of the plurality of particle-forming proteins. For example, the coronavirus antigen of the plurality of coronavirus antigens can be attached to the particle-forming protein of the plurality of particle-forming proteins through a Spy tag/SpyCatcher binding pair.

In some embodiments, the coronavirus antigen of the plurality of coronavirus antigens comprises a Spy tag at the C-terminal of the coronavirus antigen and the particle-forming protein of a plurality of particle-forming proteins comprises a SpyCatcher at the N-terminal of the particle-forming protein. In some embodiments, the coronavirus antigen of the plurality of coronavirus antigens comprises a coronavirus S protein RBD, and the coronavirus S protein RBD comprises a Spy tag at the C-terminal of the coronavirus S protein RBD and the particle-forming protein of a plurality of particle-forming proteins comprises a SpyCatcher at the N-terminal of the particle-forming protein. In some embodiments, the vaccine composition can further comprises an adjuvant. Non-limiting examples of adjuvant include: aluminum hydroxide, alhydrogel, Adda Vax, MF59, AS03, Freund's adjuvant, Montanide ISA51, CpG, Poly I:C, glucopyranosyl lipid A, flagellin, resiquimod, and a combination thereof.

Disclosed herein includes a method of stimulating an immune response in a subject in need thereof. The method comprises, for example, administering to the subject a pharmaceutically effective amount of any one of the vaccine compositions disclosed herein, thereby stimulating an immune response in the subject. Disclosed herein includes a method for treating or preventing a coronavirus infection in a subject in need thereof. The method can, for example, comprises: administering to the subject a pharmaceutically effective amount of any one of the vaccine compositions disclosed herein, thereby treating or preventing the coronavirus infection in the subject. Also disclosed herein includes a method for treating or preventing a disease or disorder caused by a coronavirus infection in a subject in need thereof. The method can, for example, comprise: administering to the subject a pharmaceutically effective amount of any one of the vaccine compositions disclosed herein, thereby treating or preventing the disease or disorder caused by the coronavirus infection in the subject. The vaccine composition can be administered to the subject, for example, once, two, three, four, or more times within two weeks, four weeks, one month, two months, three months, six months, a year, two years, three years, or more. The vaccine composition can be administered to the subject, for example, once, two, three, four, or more times at least two weeks, four weeks, one month, two months, three months, six months, a year, two years, three years, or more, apart.

In some embodiments, administering the vaccine composition induces neutralizing responses against coronaviruses different from the first coronavirus and the second coronavirus. In some embodiments, administering the vaccine composition induces neutralizing responses against additional coronaviruses different from the coronaviruses the plurality of coronavirus antigens are of. In some embodiments, administering the vaccine composition induces neutralizing responses against the coronaviruses the plurality of coronavirus antigens are of.

In some embodiments, administering the vaccine composition results in treating or preventing infection caused by a coronavirus different from the first coronavirus and the second coronavirus. In some embodiments, administering the vaccine composition results in treating or preventing infection caused by additional coronaviruses different from the coronaviruses the plurality of coronavirus antigens are of. In some embodiments, administering the vaccine composition results in treating or preventing infection caused by the coronaviruses the plurality of coronavirus antigens are of.

In some embodiments, administering the vaccine composition results in treating or preventing the disease or disorder caused by a coronavirus different from the first coronavirus and the second coronavirus. In some embodiments, administering the vaccine composition results in treating or preventing the disease or disorder caused by additional coronaviruses different from the coronaviruses the plurality of coronavirus antigens are of. In some embodiments, administering the vaccine composition results in treating or preventing the disease or disorder caused by the coronaviruses the plurality of coronavirus antigens are of.

Administering the vaccine composition can, for example, comprise administering to the subject a first vaccine composition and administering to the subject a second vaccine composition. In some embodiments, administering to the subject the second vaccine composition occurs about two, three, four weeks, or more, after administering to the subject the first vaccine composition.

Also disclosed herein include a kit comprising any of the vaccine compositions disclosed herein. In some embodiments, the kit comprises a manual providing instructions for one or more of (1) stimulating an immune response in a subject in need thereof, (2) treating or preventing a coronavirus infection in a subject in need thereof, and (3) treating or preventing a disease or disorder caused by a coronavirus infection in a subject in need thereof.

### BRIEF DESCRIPTION OF THE DRAWINGS

The patent or application file contains at least one drawing executed in color. Copies of this patent or patent application publication with drawing(s) will be provided by the Office upon request and payment of the necessary fee.

FIG. 1A-FIG. 1D depict non-limiting exemplary embodiments of RBDs chosen for this study. FIG. 1A left shows an exemplary structure of SARS-CoV-2 S trimer (PDB 6VXX) with one RBD (dashed circle) in an “up” position. FIG. 1A middle and right shows non-limiting exemplary data related to sequence conservation of 12 RBDs calculated by the ConSurf Database plotted on a surface representation of the RBD structure (PDB 7BZ5). Epitopes for representatives from defined classes of RBD-binding antibodies (class 1-class 4) indicated by dashed lines. FIG. 1B depicts non-limiting exemplary embodiments of the viral strains from which the 12 Sarbecovirus RBDs were derived. FIG. 1C shows a non-limiting exemplary phylogenetic tree of human and selected other coronaviruses based on RBD protein sequences. Red shading indicates strains known to use ACE2 as a receptor. FIG. 1D depicts a non-limiting exemplary heat map showing percent amino acid sequence identities between 12 Sarbecovirus RBDs.

FIG. 2A-FIG. 2C depict non-limiting exemplary embodiments of construction of RBD nanoparticles. FIG. 2A depicts non-limiting exemplary embodiments of SpyTagged RBDs attached to SpyCatcher003-mi3 to make a homotypic particle and three mosaic particles. 10 of 60 potential coupling sites on mi3 are shown for clarity. FIG. 2B shows non-limiting exemplary data of a SEC profile showing separation of RBD nanoparticles and free RBD proteins. FIG. 2C shows non-limiting exemplary data of coomassie-stained SDS-PAGE of RBD-coupled nanoparticles, free RBD proteins, and uncoupled SpyCatcher003-mi3 particles (SC3-mi3).

FIG. 3A-FIG. 3F depict non-limiting exemplary data showing RBD nanoparticles induce cross-reactive IgG responses in immunized mice. Red and gray rectangles below ELISA and neutralization data represent mismatched strains (red; RBD from that strain was not present on the immunized particle) or matched strains (gray; RBD was present on the immunized particle). FIG. 3A depicts a nonlimiting exemplary embodiment of the immunization schedule. Adjuvant=AddaVax (Invivogen). FIG. 3B-FIG. 3F depict non-limiting exemplary data showing mice that were immunized with soluble SARS-CoV-2 S trimer (SARS-2 S; brown bars), or the following nanoparticles: homotypic SARS-2 (red), mosaic-4a (green), mosaic-4b (yellow), mosaic-8 (blue), or unconjugated SpyCatcher003-mi3 (mi3; black). ELISA data from serum IgG responses to SARS-2 spike trimer (FIG. 3B) or RBDs (FIG. 3C-FIG. 3F) shown as area under the curve (AUC). For FIG. 3C-FIG. 3E, neutralization potencies are presented as half-maximal inhibitory dilutions (ID₅₀ values) of sera against the pseudoviruses from the indicated coronavirus strains. Dashed horizontal lines correspond to the lowest dilution representing the limit of detection. Each dot represents serum from one animal, with means and standard deviations for vaccinated cohorts represented by rectangles (mean) and horizontal lines (SD). Significant differences between groups linked by horizontal lines are indicated by asterisks and p-values. NS=not significant. (FIG. 3B-FIG. 3F) Neutralization and/or binding data for serum IgGs for recognition of (FIG. 3B) SARS-2 spike trimer, (FIG. 3C) SARS-2 RBD and SARS-2 pseudovirus, (FIG. 3D) SHC014 and WIV1 RBDs and corresponding pseudoviruses, (FIG. 3E) SARS RBD and SARS pseudovirus, (FIG. 3F) Yun11, BM-4831, and BtKY72 RBDs.

FIG. 4A-FIG. 4H depict non-limiting exemplary data showing IgGs from convalescent COVID-19 plasma show little to no cross-reactive responses. FIG. 4A-FIG. 4F show non-limiting exemplary data related to plasma IgG responses evaluated by ELISA (data shown as binding curves with plasma names listed) against RBDs from (FIG. 4A) SARS-2, (FIG. 4B) RaTG13, (FIG. 4C) SHC014, (FIG. 4D) WIV1, (FIG. 4E) Rs4081, and (FIG. 4F) BM-4831. Data points are plotted as the mean and standard deviation of duplicate measurements. IOMA, an anti-HIV-1 IgG, was used as a control. FIG. 4G shows non-limiting exemplary data related to ELISA results from FIG. 4A-FIG. 4F presented as area under the curve (AUC), where each dot represents one plasma sample, with means and standard deviations represented by rectangles (mean) and horizontal lines (SD). Significant differences between groups linked by horizontal lines are indicated by asterisks and p-values. FIG. 4H shows non-limiting exemplary data related to IC₅₀ values for pseudotyped neutralization assays using IgGs from COV7, COV21, and COV72 plasmas (evaluated at top concentrations of 1500 μg/mL) against the indicated strains. Mean=arithmetic mean IC₅₀; SD=standard deviation.

FIG. 5 shows non-limiting exemplary data related to alignment of RBD sequences used for making mosaic particles. Sequences shown are for the RBDs of SARS-CoV-2 (SARS-2, GenBank: MN985325.1, SEQ ID NO: 15), RaTG13 (QHR63300, SEQ ID NO: 16), SHC014 (RsSHC014, KC881005, SEQ ID NO: 17), Rs4081 (KY417143, SEQ ID NO: 18), PCoV_GX-P5L (pang17) (QIA48632, SEQ ID NO: 19), RmYN02 (GSAID EPI_ISL_412977, SEQ ID NO: 20), Rf1 (DQ412042, SEQ ID NO: 21), WIV1 (KF367457, SEQ ID NO: 22), SARS-CoV (AAP13441.1, SEQ ID NO: 23), Yun11 (Cp/Yunnan2011, JX993988, SEQ ID NO: 24), BM-4831 (BM48-31/BGR/2008, NC014470, SEQ ID NO: 25), and BtKY72 (KY352407, SEQ ID NO: 26). SARS-2 RBD residues that interact directly with ACE2 are indicated by an asterisk. Note that antibody neutralization by direct binding of ACE2-binding residues does not represent the only mechanism of neutralization for ACE2-tropic viruses. This has been shown for monoclonal human antibodies derived from COVID-19 patients: in some embodiments, neutralizing antibodies do not directly interact with the ACE2-binding site on RBD (for example, class 3 anti-SARS-CoV-2 neutralizing antibodies). Red shading indicates strains known to use ACE2 as a receptor.

FIG. 6A-FIG. 6B depict non-limiting exemplary data showing RBDs from the eight Sarbecovirus S proteins conjugate equivalently to SpyCatcher003-mi3, suggesting a statistical mixture of RBDs on mosaic particles. FIG. 6A depicts non-limiting exemplary data of SEC profiles showing separation of RBD nanoparticles and free RBD proteins. FIG. 6B depicts non-limiting exemplary data showing coomassie-stained SDS-PAGE of RBD-coupled nanoparticles, free RBD proteins, and uncoupled SpyCatcher003-mi3 particles (SC3-mi3).

FIG. 7A-FIG. 7F show non-limiting exemplary data related to day 14 serum IgG responses to RBDs evaluated by ELISA shown as area under the curve (AUC) from mice immunized with soluble SARS-CoV-2 S trimers (SARS-2 S) or RBDs on nanoparticles (homotypic SARS-2, mosaic-4a, mosaic-4b, mosaic-8, or unconjugated SpyCatcher003-mi3 (mi3)). Each dot represents serum from one animal, with means and standard deviations represented by rectangles (mean) and horizontal lines (SD). RBDs from strains that were not present on an immunized particle or were present on an immunized particle are indicated by red and gray rectangles, respectively, below the ELISA data. Significant differences between groups linked by horizontal lines are indicated by asterisks and p-values. NS=not significant. FIG. 7A-FIG. 7B depict non-limiting exemplary data related to binding of serum IgGs to (FIG. 7A) Rs4081 and (FIG. 7B) RaTG13 RBDs. FIG. 7C shows non-limiting exemplary data related to binding of serum IgGs to SARS-2 RBD (top left), a triple RBD mutant in a mouse-adapted SARS-CoV-2 that includes substitutions adjacent to the N501Y RBD mutation in an emergent UK SARS-CoV-2 lineage (https://virological.org/t/preliminary-genomic-characterisation-of-an-emergent-sars-cov-2-lineage-in-the-uk-defined-by-a-novel-set-of-spike-mutations/563) (top right), and Y453F, the “Danish mink variant” (bottom left). FIG. 7D depicts non-limiting exemplary data related to binding of serum IgGs to RBD from MERS-CoV (a non-ACE2-binding merbecovirus, representing a different subgenus from Sarbecoviruses). FIG. 7E-FIG. 7F show non-limiting exemplary data related to comparison of ELISA data for serum binding to selected RBDs presented as AUC, endpoint titers, midpoint titers, or binding curves. Day 14 serum IgG responses to (FIG. 7E) SARS-2 or (FIG. 7F) SARS RBDs evaluated by ELISA shown as AUC (top left), endpoint titers (top middle), midpoint (EC₅₀) titers (top right), or binding curves (bottom left). For AUC, each dot represents serum from one animal, with means and standard deviations represented by rectangles (mean) and horizontal lines (SD). For endpoint and midpoint titers, each dot represents serum from one animal, with geometric means and geometric standard deviations represented by rectangles (mean) and horizontal lines (SD). Binding curves are shown with data points representing the mean and SD of duplicate measurements fit to a binding model (see Materials and Methods of Example 1 below) for animals immunized with mosaic-8 and homotypic SARS-2.

FIG. 8A-FIG. 8D depict non-limiting exemplary data related to the correlation of ELISA and neutralization titers. Spearman correlation coefficients (rs) and p-values shown for graphs of anti-RBD ELISA titers (AUC) versus pseudovirus neutralization ID₅₀ values; significance indicated as asterisks. (FIG. 8A) SARS-2. (FIG. 8B) SARS. (FIG. 8C) SHC014. (FIG. 8D) WIV1.

FIG. 9A-FIG. 9E depict non-limiting exemplary data related to antigen-specific IgG⁺ B-cell analysis of splenocytes isolated from animals immunized with mosaic-RBD nanoparticles. FIG. 9A depicts non-limiting exemplary embodiments of a flow cytometry gating strategy for characterizing RBD-specific IgG B-cells isolated from splenocytes. Anti-CD4, anti-CD8, anti-F4/80, anti-Ly6G, and anti-IgM were used in the dump to remove T-cells, macrophages, and IgM⁺ B-cells. Antigen-specific IgG⁺ B-cells were isolated using labeled anti-CD19 and anti-IgG antibodies, and probed for binding RBD with a pair of fluorophore-conjugated RBD tetramers (SARS-2 RBD and Rs4081 RBD). FIG. 9B depicts non-limiting exemplary data related to flow cytometry analysis for antigen-specific IgG⁺ splenocytes isolated from animals immunized with mosaic-RBD particles. The 4-way gate shown for each animal separates each population of RBD single-positive and double-positive cells and was used for the % antigen-specific populations shown in FIG. 9C-FIG. 9E. Q1 represents the Rs4081 RBD⁺ population, Q2 represents the Rs4081 RBD⁺/SARS-2 RBD⁺ population, Q3 represents the SARS-2 RBD⁺ population, and Q4 represents the RBD⁻ population. FIG. 9C-FIG. 9E show non-limiting exemplary data related to the percent single-positive (SP) and double-positive (DP) cells for the indicated groups. Significant differences between groups linked by horizontal lines are indicated by asterisks and p-values. NS=not significant. (FIG. 9C) Percent SARS-2 RBD⁺ B-cells within the IgG⁺ B-cell population. (FIG. 9D) Percent Rs4081 RBD⁺ B-cells within the IgG⁺ B-cell population. (FIG. 9E) Percent SARS-2 RBD⁺/Rs4081 RBD⁺ B-cells within the IgG⁺ B-cell population.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

SARS-CoV-2, a newly-emergent Betacoronavirus, resulted in a global pandemic in 2020, infecting millions and causing the respiratory disease COVID-19. Two other zoonotic Betacoronaviruses, SARS-CoV and MERS-CoV, also resulted in outbreaks within the last 20 years. All three viruses presumably originated in bats, with SARS-CoV and MERS-CoV adapting to intermediary animal hosts before jumping to humans. SARS-like viruses circulate in bats and serological surveillance of people living near caves where bats carry diverse coronaviruses demonstrates direct transmission of SARS-like viruses with pandemic potential, suggesting a pan-coronavirus vaccine is needed to protect against future outbreaks and pandemics. In particular, the bat WIV1 and SHC014 strains are thought to represent an ongoing threat to humans.

Although existing vaccines may offer protection against one type of coronavirus such as SARS-CoV-2, coronavirus reservoirs in bats suggest future cross-species transmission, necessitating a vaccine that protects against emerging coronaviruses as well as SARS-CoV-2. There is a strong and urgent need for effective and longer-lasting vaccines that can elicit higher tiers of neutralizing antibodies and induce heterologous binding and neutralizing responses against a broad spectrum of coronaviruses. In addition to the need for a vaccine against SARS-CoV-2, there is an also urgent need to protect against related zoonotic coronaviruses that could spill-over into humans to cause future pandemics. Disclosed herein includes immunization compositions and method for protecting against infection from potentially emerging animal coronaviruses as well as against SARS-CoV-2.

As described herein, using for example a “plug and display” approach, a series of nanoparticles were made displaying the receptor-binding domain (RBD) of either SARS-CoV-2 alone (homotypic nanoparticles) or co-displaying the SARS-CoV-2 RBD along with a diverse set of RBDs from bat and pangolin Betacoronaviruses that represent threats to humans (mosaic nanoparticles). Going beyond recent reports describing autologous responses to SARS-CoV-2 homotypic nanoparticles (e.g., responses against SARS-CoV-2 virus after injection of SARS-CoV-2 nanoparticles), homotypic nanoparticle immunization produces responses that both bind and neutralize heterologous coronaviruses after boosting. Co-display of other RBDs along with the SARS-CoV-2 RBD does not diminish reactivity compared with responses to a homotypic nanoparticle, thereby showing there would be no disadvantages in using mosaic nanoparticles to protect against SARS-CoV-2. The advantage of mosaic RBD over homotypic SARS-CoV-2 nanoparticles is mosaic nanoparticles elicited antibodies with superior cross-reactive recognition of heterologous RBDs compared to immunizations with homotypic SARS-CoV-2-RBD-nanoparticles; in particular because cross-reactive responses were observed after only a prime with mosaic nanoparticles, whereas cross-reactive responses to the homotypic particle required both prime and boost injections. Inducing neutralizing responses after only a single injection would be a great advantage for a vaccine that needs to be delivered to millions/billions of people. In some embodiments, the type of mosaic nanoparticle described here can be adapted to present RBDs from new zoonotic coronaviruses as they are discovered, such that vaccine-induced recognition of zoonotic Sarbecoviruses provides protection from future coronavirus-related pandemic(s) while also being used for protection against SARS-CoV-2.

As described herein, IgGs from COVID-19 convalescent human plasmas were evaluated, showing that they exhibited little to no recognition of coronaviruses other than SARS-CoV-2. Thus infection-induced immunity in humans would unlikely to be protective against an outbreak of a new zoonotic coronavirus, another indication for the need to develop a vaccine to protect against emerging Sarbecoviruses.

Disclosed herein include a multivalent carrier and related vaccine composition, methods and kits. The multivalent carrier and the vaccine composition thereof can simultaneously present coronavirus antigens from human and animal coronaviruses of different taxonomical groups and elicit cross-reactive antibody responses. The multivalent carrier and the vaccine composition thereof can elicit substantially enhanced and broad binding and neutralizing responses, in contrast to plasma antibodies elicited in humans by, for example, SARS-CoV-2 infection. The multivalent carrier and the vaccine composition thereof also demonstrate enhanced heterologous binding and neutralization properties against human and bat Beta-coronaviruses (e.g., Sarbecoviruses) compared with homotypic nanoparticles (e.g., homotypic SARS-CoV-2 nanoparticles).

Disclosed herein includes a multivalent carrier. The multivalent carrier comprises a plurality of coronavirus antigens, the plurality of coronavirus antigens comprising at least a first coronavirus antigen of a first coronavirus and a second coronavirus antigen of a second coronavirus that is different from the first coronavirus. Disclosed herein also includes a vaccine composition comprising the multivalent carrier herein described.

Disclosed herein includes a method of stimulating an immune response in a subject in need thereof. In some embodiments, the method comprises administering to the subject a pharmaceutically effective amount of the vaccine composition described herein, thereby stimulating an immune response in the subject. Disclosed herein also includes a method of treating or preventing a coronavirus infection in a subject in need thereof. In some embodiments, the method comprises administering to the subject a pharmaceutically effective amount of the vaccine composition described herein, thereby treating or preventing the coronavirus infection in the subject. In addition, a method of treating or preventing a disease or disorder caused by a coronavirus infection in a subject in need thereof is disclosed herein. In some embodiments, the method comprises administering to the subject a pharmaceutically effective amount of the vaccine composition described herein, thereby treating or preventing the disease or disorder caused by the coronavirus infection in the subject.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, the terms “antigen” or “immunogen” are used interchangeably to refer to a substance, typically a protein, which is capable of inducing an immune response in a subject (e.g. a mammal, such as a human). The term also refers to proteins that are immunologically active in the sense that once administered to a subject, either directly or in the form of a nucleotide sequence or vector that encodes the protein, is able to evoke an immune response of the humoral and/or cellular type directed against that protein or a variant thereof.

As used herein, “sequence identity” or “identity” in the context of two nucleic acid or polypeptide sequences makes reference to the nucleotide bases or residues in the two sequences that are the same when aligned for maximum correspondence over a specified comparison window. Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; Pearson et al., Meth. Mol. Bio. 24:307-31, 1994; and Altschul et al., J. Mol. Biol. 215:403-10, 1990 (the content of each of these references is incorporated herein in its entirety).

When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule. A functionally equivalent residue of an amino acid used herein typically can refer to other amino acid residues having physiochemical and stereochemical characteristics substantially similar to the original amino acid. The physiochemical properties include water solubility (hydrophobicity or hydrophilicity), dielectric and electrochemical properties, physiological pH, partial charge of side chains (positive, negative or neutral) and other properties identifiable to one of skill in the art. The stereochemical characteristics include spatial and conformational arrangement of the amino acids and their chirality. For example, glutamic acid is considered to be a functionally equivalent residue to aspartic acid in the sense of the current disclosure. Tyrosine and tryptophan are considered as functionally equivalent residues to phenylalanine. Arginine and lysine are considered as functionally equivalent residues to histidine.

The term “substantially identical” as used herein in the context of two or more sequences refers to a specified percentage of amino acid residues or nucleotides that are identical or functionally equivalent, such as about, at least or at least about 65% identity, optionally, about, at least or at least about 70%, 75%, 80%, 85%, 90%, 95%, or 99% identity over a specified region or over the entire sequence.

As used herein, the term “variant” refers to a polynucleotide or polypeptide having a sequence substantially similar or identical to a reference (e.g., the parent) polynucleotide or polypeptide. In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least, or at least about, 50%, 55%, 60%, 65%, 70%, 75%, 80%, 85%, 90%, 91%, 92%, 93%, 94%, 95%, 96%, 97%, 98%, 99%, or more sequence identity to the reference polynucleotide as determined by sequence alignment programs known in the art. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. A variant of a polypeptide can have, for example, at least, or at least about, 60%, 65%, 70%, 75%, 80%, 85%, 90%, 91%, 92%, 93%, 94%, 95%, 96%, 97%, 98%, 99%, or more sequence identity to the reference polypeptide as determined by sequence alignment programs known in the art.

Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those commonly known and used in the art. Standard techniques can be used for chemical syntheses, chemical analyses, pharmaceutical preparation, formulation, and delivery, and treatment of patients.

### Coronaviruses and Coronavirus Antigens

The multivalent carriers, and related vaccine composition, methods and kits herein described comprise a plurality of heterologous coronavirus antigens. The term “heterologous antigens” means that the antigens are of different origins, such as derived from pathogens of different taxonomic groups such as different strains, species, subgenera, genera, subfamilies or families and/or from antigenically divergent pathogens (e.g., variants thereof). Multivalent carriers can also be referred to as mosaic carriers. Classification of viruses into various taxonomic groups is well understood by those skilled in the art. In the embodiments herein described, the heterologous coronavirus antigens of the multivalent carriers herein described are derived from coronaviruses of different strains, species, subgenera, genera, and/or subfamilies in the Coronaviridae family.

The term “coronavirus” as used herein refers to a virus in the family Coronaviridae, which is in turn classified within the order Nidovirales. The coronaviruses are large, enveloped, positive-stranded RNA viruses. The coronaviruses have the largest genomes of the RNA viruses known in the art and replicate by a unique mechanism that results in a high frequency of recombination. The coronaviruses include antigenic groups I, II, and III. Nonlimiting examples of coronaviruses include SARS coronavirus (e.g., SARS-CoV and SARS-CoV-2), MERS coronavirus, transmissible gastroenteritis virus (TGEV), human respiratory coronavirus, porcine respiratory coronavirus, canine coronavirus, feline enteric coronavirus, feline infectious peritonitis virus, rabbit coronavirus, murine hepatitis virus, sialodacryoadenitis virus, porcine hemagglutinating encephalomyelitis virus, bovine coronavirus, avian infectious bronchitis virus, and turkey coronavirus, as well as chimeras thereof. Additional information related to coronavirus including classification, virion structure, genome structure, genetics and pathology is described, for example, in KV Holmes, Encyclopedia of Virology, 1999: 291-298, the content of which is incorporated herein by reference.

In some embodiments, a coronavirus described herein is in the genus of Alpha-coronavirus and the coronavirus antigens can be of or derived from any species or strains in the genus of Alpha-coronavirus. In some embodiments, a coronavirus described herein is in the genus of Beta-coronavirus and the coronavirus antigens can be of or derived from any species or strains in the genus of Beta-coronavirus. Member viruses in the genus of Alpha-coronavirus and Beta-coronavirus are enveloped, positive-strand RNA viruses that can infect mammals.

A coronavirus described herein can be of any subgenus of Alpha-coronavirus genus, including but not limited to Colacovirus (e.g. Bat coronavirus CDPHE15), Decacovirus (e.g. Bat coronavirus HKU10 and Rhinolophus ferrumequinum Alphacoronavirus HuB-2013), Duvinacovirus (Human coronavirus 229E), Luchacovirus (e.g. Lucheng Rn rat coronavirus), Minacovirus (e.g. Mink coronavirus 1), Minunacovirus (e.g. Miniopterus bat coronavirus 1 and Miniopterus bat coronavirus HKU8), Myotacovirus (e.g. Myotis ricketti Alphacoronavirus Sax-2011), Nyctacovirus (e.g. Nyctalus velutinus Alphacoronavirus SC-2013 and Pipistrellus kuhlii coronavirus 3398), Pedacovirus (e.g. Porcine epidemic diarrhea virus and Scotophilus bat coronavirus 512), Rhinacovirus (e.g. Rhinolophus bat coronavirus HKU2), Setracovirus (e.g. Human coronavirus NL63 and NL63-related bat coronavirus strain BtKYNL63-9b), Soracovirus (e.g. Sorex araneus coronavirus T14), Sunacovirus (e.g. Suncus murinus coronavirus X74), and Tegacovirus (e.g. Alphacoronavirus 1).

Within the genus Beta-coronavirus, five subgenera or lineages have been recognized, including Embecovirus (lineage A), Sarbecovirus (lineage B), Merbecovirus (lineage C), Nobecovirus (lineage D), and Hibecovirus. Accordingly, in some embodiments, a coronavirus described herein can be any strain or species in any of the subgenera or lineages of Beta-coronavirus.

For example, a coronavirus antigen can be of or derived from any species or strains in the subgenus of Embecovirus, including but not limited to Beta-coronavirus 1 (e.g. Bovine coronavirus and human coronavirus OC43), China Rattus coronavirus HKU24, Human coronavirus HKU1, Murine coronavirus (e.g. mouse heptatitis virus), and Myodes coronavirus 2JL14. The coronavirus antigen can be of or derived from any species or strains in the subgenus of Sarbecovirus, including but not limited to SARS-CoV, SARS-CoV2, 16BO133, Bat SARS CoV Rf1, Bat coronavirus HKU3 (BtCoV HKU3), LYRa11, Bat SARS-CoV/Rp3, Bat SL-CoV YNLF_31C, Bat SL-CoV YNLF_34C, SHC014-CoV, WIV1, WIV16, Civet SARS-CoV, Rc-o319, SL-ZXC21, SL-ZC45, Pangolin SARSr-COV-GX, Pangolin SARSr-COV-GD, RshSTT182 , RshSTT200, RacCS203, RmYN02, RpYN06, RaTG13, Bat CoV BtKY72, and Bat CoV BM48-31. The coronavirus antigen can be of any species or strains in the subgenus of Merbecovirus, including but not limited to Hedgehog coronavirus 1, MERS-CoV, Pipistrellus bat coronavirus HKU5, and Tylonycteris bat coronavirus HKU4. The coronavirus antigen can be of any species or strains in the subgenus of Nobecovirus, including but not limited to Eidolon bat coronavirus C704, Rousettus bat coronavirus GCCDC1, and Rousettus bat coronavirus HKU9. The coronavirus antigen can be of any species or strains in the subgenus of Hibecovirus, including but not limited to Bat Hp-Betacoronavirus Zhejiang 2013.

The coronaviruses described herein can be, for example, phylogenetically clustered in functionally distinct clades. For example, the coronaviruses of lineage B Beta-coronavirus (Sarbecovirus) can be clustered into clade 1, clade 2, clade 1/2, or clade 3 using the nucleotide sequences of nonstructural protein gene ORF1a and ORF1b (see, for example, Hu et al., PLOS Pathog 13(11): e1006698). Accordingly, the coronavirus antigens can be of or derived from any species or strain in any one of these clades. For example, the coronavirus antigens can be of any species or strain in clade 1, including but not limited to SARS-CoV, WIV1, LYRa11, Rs7327, Rs4231, Rs4084, and SHC014. The coronavirus antigens can be of any species or strain in clade 2, including but not limited to As6526, Yunnan 2011, Shaanxi 2011, 279-2005, Rs4237, Rs4081, Rp3, Rs4247, HKU3-8, HKU3-13, GX2013, Longquan-140, YN2013, Rf4092, ZXC21, ZC45, JL2012, HuB2013, Rf1, HeB2013, and 273-2005. The coronavirus antigens can be of any species or strain in clade 1/2, including but not limited to SARS-CoV2. The coronavirus antigens can be of any species or strain in clade 3, including but not limited to BM48-31. The coronavirus antigen described herein can be of a coronavirus, for example, SARS, SARS-2, WIV1, SHC014, Rf1, RmYN02, pang17, RaTG13, and Rs4081.

As exemplified herein, SARS virus (e.g., SARS-CoV and SARS-CoV-2) is an enveloped coronavirus carrying a single-stranded positive-sense RNA genome (˜30 kb), belonging to the genus Betacoronavirus from the Coronaviridae family. The virus RNA encodes four structural proteins including spike (S), envelope (E), membrane (M), and nucleocapsid (N) proteins, 16 non-structural proteins, and nine accessory proteins. The S glycoprotein contains an ectodomain that can be processed into S1 and S2 subunits, a transmembrane domain, and an intracellular domain. Both SARS-CoV and SARS-CoV-2 bind the human ACE2 via the receptor binding domain within the S1 subunit to facilitate entry into host cells, followed by membrane fusion mediated by the S2 subunit.

A coronavirus antigen of a coronavirus herein described can be any of a variety of coronavirus proteins capable of inducing an immune response against a coronavirus. Suitable coronavirus antigens are those that can elicit a protective immune response, such as producing broadly neutralizing antibodies. For example, the coronavirus antigen can comprise a coronavirus spike(S) protein, spike receptor binding domain (RBD), S1 subunit, S2 subunit, spike full ectodomain proteins, papain-like proteases, 3CL proteases, nucleocapsid proteins, envelope proteins, membrane proteins, or any of the structural, non-structural or accessory proteins that form a coronavirus.

In some embodiments, a coronavirus antigen used herein comprises a spike (S) protein or a portion thereof. A S protein is one of four major structural proteins covering the surface of each virion. The S protein, comprising a S1 subunit and a S2 subunit, is a highly glycosylated, type I transmembrane protein capable of binding to a host-cell receptor and mediates viral entry. The S protein comprises a domain referred to as the RBD that mediates the interaction with the host-cell receptor to enter the host cell after one or more RBDs adopts an “up” position to bind the host receptor. It is believed that after binding the receptor, a nearby host protease cleaves the spike, which releases the spike fusion peptide, facilitating virus entry. Known host receptors for coronaviruses (e.g., Beta-coronaviruses) include antiotensin-converting enzyme 2 (ACE2), dipeptidyl peptidase-4 (DPP4) or sialic acids. For example, the RBDs of human coronaviruses SARS-CoV-2, SARS-CoV, HCoV-NL63, and related animal coronaviruses (WIV1 and SCH014) use ACE2 as their host receptor, while MERS-CoV uses DPP4 as its host receptor.

The coronavirus antigen used herein can, for example, comprise a coronavirus nucleocapsid protein (N protein) or a portion thereof. The N protein is a multifunctional RNA-binding protein required for viral RNA transcription, replication, and packaging. The N protein consists of three domains, an N-terminal RNA-binding domain, a central intrinsically disordered region, followed by a C-terminal dimerization domain. The RNA-binding domain contains multiple positively charged binding surfaces that form charged interactions with RNA promoting its helical arrangement. The coronavirus antigen used herein can comprise any of these N protein domains or a portion thereof.

In some embodiments, the coronavirus antigen used herein comprises a coronavirus membrane protein (M protein) or a portion thereof. The M protein is the most abundant structural protein and defines the shape of the viral envelope. The M protein is regarded as the central organizer of the viral assembly, interacting with other major coronaviral structural proteins.

In some embodiments, the coronavirus antigen used herein comprises a coronavirus envelope protein (E protein) or a portion thereof. The E protein is a small membrane protein and minor component of the virus particles. Without being bound to any theory, it is believed that the E protein plays roles in virion assembly and morphogenesis, alteration of the membrane of host cells and virus-host cell interaction.

In some embodiments, the coronavirus antigen used herein comprises a coronavirus hemagglutinin-esterase protein (HE protein) or a portion thereof. The HE protein, which is another envelope protein, mediates reversible attachment to O-acetylated sialic acids by acting both as lectins and receptor-destroying enzymes.

In some embodiments, the coronavirus antigen used herein comprises a coronavirus papain-like protease or a portion thereof. The coronavirus papain-like protease is one of several nonstructural proteins, and is responsible for processing of viral proteins into functional, mature subunits during maturation. For example, the coronavirus papain-like protease can cleave a site at the amino-terminal end of the viral replicase region. In addition to its role in viral protein maturation, papain-like protease exhibits both a deubiquitinating and deISG15ylating activity. In vivo, this protease antagonizes innate immunity by acting on IFN beta and NF-kappa B signaling pathways.

In some embodiments, the coronavirus antigen used herein comprises a coronavirus 3CL protease or a portion thereof. The 3CL protease is another main protease in addition to the papain-like protease and is required for processing of viral polypeptides into distinct, functional proteins. In some embodiments, the 3CL protease is a SARS-CoV-2 3CL Protease, which is a C30-type cysteine protease located within the non-structural proteins 3 (NS3) region of the viral polypeptide. Analysis of the Coronavirus genome reveals at least 11 sites of cleavage for the 3CL protease, many containing the amino acid sequence LQ [S/A/G].

The coronavirus antigen disclosed herein can, in some embodiments, comprise a S protein or a portion thereof, a N protein or a portion thereof, a HE protein or a portion thereof, a papain-like protease or a portion thereof, a coronavirus 3CL protease or a portion thereof, a M protein or a portion thereof, or a combination thereof.

In some embodiments, the coronavirus antigen can be an immunogenic portion of a coronavirus protein herein described. It will be appreciated by those skilled in the art that an immunogenic portion of a coronavirus antigen can be fragments of the S protein (e.g., spike protein RBD), N protein, HE protein, papain-like protease, 3CL protease, or M protein capable of eliciting an immune response against one or more coronaviruses. The immunogenic portion can comprise about, at least or at least about, at most or at most about, 50, 60, 70, 80, 90, 100, 110, 120, 130, 140, 150, 160, 170, 180, 190, 200, 210, 220, 230, 240, 250, or a number or a range between any two of these values, contiguous amino acid residues from the coronavirus proteins. In some embodiments, the immunogenic portion comprises a S protein RBD or a portion thereof. The portion of the S protein RBD can comprise the receptor binding motif of the S protein RBD.

In some embodiments, the coronavirus antigen is a S protein or a portion thereof comprising or consisting of an amino acid sequence having, having about, having at least, or having at least about, 80%, 85%, 90%, 95%, 98%, 99%, or more sequence identity to the amino acid sequence of any of the coronavirus S proteins from one or more coronaviruses selected from SARS-CoV, SARS-CoV2, WIV1, LYRa11, Rs7327, Rs4231, Rs4084, SHC014, As6526, Yunnan 2011, Shaanxi 2011, 279-2005, Rs4237, Rs4081, Rp3, Rs4247, HKU3-8, HKU3-13, GX2013, Longquan-140, YN2013, Rf4092, ZXC21, ZC45, JL2012, HuB2013, Rf1, HeB2013, 273-2005, and BM48-31, optionally selected from SARS, SARS-2, WIV1, SHC014, Rf1, RmYN02, pang17, RaTG13, and Rs4081.

In some embodiments, the coronavirus antigen is a N protein or a portion thereof comprising or consisting of an amino acid sequence having, having about, having at least, or having at least about, 80%, 85%, 90%, 95%, 98%, 99% or more, sequence identity to the amino acid sequence of any of the coronavirus N proteins from one or more coronaviruses selected from SARS-CoV, SARS-CoV2, WIV1, LYRa11, Rs7327, Rs4231, Rs4084, SHC014, As6526, Yunnan 2011, Shaanxi 2011, 279-2005, Rs4237, Rs4081, Rp3, Rs4247, HKU3-8, HKU3-13, GX2013, Longquan-140, YN2013, Rf4092, ZXC21, ZC45, JL2012, HuB2013, Rf1, HeB2013, 273-2005, and BM48-31, optionally selected from SARS, SARS-2, WIV1, SHC014, Rf1, RmYN02, pang17, RaTG13, and Rs4081.

In some embodiments, the coronavirus antigen is a HE protein or a portion thereof comprising or consisting of an amino acid sequence having, having about, having at least, having at least about, 80%, 85%, 90%, 95%, 98%, 99% or more, sequence identity to the amino acid sequence of any of the coronavirus HE proteins from one or more coronaviruses selected from SARS-CoV, SARS-CoV2, WIV1, LYRa11, Rs7327, Rs4231, Rs4084, SHC014, As6526, Yunnan 2011, Shaanxi 2011, 279-2005, Rs4237, Rs4081, Rp3, Rs4247, HKU3-8, HKU3-13, GX2013, Longquan-140, YN2013, Rf4092, ZXC21, ZC45, JL2012, HuB2013, Rf1, HeB2013, 273-2005, and BM48-31, optionally selected from SARS, SARS-2, WIV1, SHC014, Rf1, RmYN02, pang17, RaTG13, and Rs4081.

In some embodiments, the coronavirus antigen is a papain-like protease or a portion thereof comprising or consisting of an amino acid sequence having, having about, having at least, having at least about, 80%, 85%, 90%, 95%, 98%, 99% or more, sequence identity to the amino acid sequence of any of the coronavirus papain-like proteases from one or more coronaviruses selected from SARS-CoV, SARS-CoV2, WIV1, LYRa11, Rs7327, Rs4231, Rs4084, SHC014, As6526, Yunnan 2011, Shaanxi 2011, 279-2005, Rs4237, Rs4081, Rp3, Rs4247, HKU3-8, HKU3-13, GX2013, Longquan-140, YN2013, Rf4092, ZXC21, ZC45, JL2012, HuB2013, Rf1, HeB2013, 273-2005, and BM48-31, optionally selected from SARS, SARS-2, WIV1, SHC014, Rf1, RmYN02, pang17, RaTG13, and Rs4081.

In some embodiments, the coronavirus antigen is a 3CL protease or a portion thereof comprising or consisting of an amino acid sequence having, having about, having at least, having at least about, 80%, 85%, 90%, 95%, 98%, 99% or more, sequence identity to the amino acid sequence of any of the coronavirus 3CL proteases from one or more coronaviruses selected from SARS-CoV, SARS-CoV2, WIV1, LYRa11, Rs7327, Rs4231, Rs4084, SHC014, As6526, Yunnan 2011, Shaanxi 2011, 279-2005, Rs4237, Rs4081, Rp3, Rs4247, HKU3-8, HKU3-13, GX2013, Longquan-140, YN2013, Rf4092, ZXC21, ZC45, JL2012, HuB2013, Rf1, HeB2013, 273-2005, and BM48-31, optionally selected from SARS, SARS-2, WIV1, SHC014, Rf1, RmYN02, pang17, RaTG13, and Rs4081.

In some embodiments, the coronavirus antigen is a M protein or a portion thereof comprising or consisting of an amino acid sequence having, having about, having at least, having at least about, 80%, 85%, 90%, 95%, 98%, 99% or more, sequence identity to the amino acid sequence of any of the coronavirus M proteins from one or more coronaviruses selected from SARS-CoV, SARS-CoV2, WIV1, LYRa11, Rs7327, Rs4231, Rs4084, SHC014, As6526, Yunnan 2011, Shaanxi 2011, 279-2005, Rs4237, Rs4081, Rp3, Rs4247, HKU3-8, HKU3-13, GX2013, Longquan-140, YN2013, Rf4092, ZXC21, ZC45, JL2012, HuB2013, Rf1, HeB2013, 273-2005, and BM48-31, optionally selected from SARS, SARS-2, WIV1, SHC014, Rf1, RmYN02, pang17, RaTG13, and Rs4081.

In some embodiments, a coronavirus antigen comprises a coronavirus spike RBD or a portion or a variant thereof. The coronavirus spike RBD is known to have a three-dimensional structure of a twisted five-stranded antiparallel β sheet (e.g. β1, β2, β3, β4 and β7 in SARS-CoV-2) with short connecting helices and loops that form a core. Between two β strands (e.g. β4 and β7 strands in SARS-CoV-2) in the core, there is an extended insertion containing two short β strands (e.g. β5 and β6 in SARS-CoV-2), two α helices (e.g., α4 and α5 in SARS-CoV-2) and loops connecting the two β strands and/or the two α helices. This extended insertion is referred to as receptor binding motif (RBM), which contains residues that are capable of binding to host-cell receptors (e.g., ACE2). In the embodiments herein described, the coronavirus spike RBD, and particularly RBM, is capable of recognizing and binding to a host-cell receptor in order to initiate the spike protein-mediated viral entry. Detailed information of the coronavirus spike RBD and the host-cell receptor (e.g., ACE2) interface and related contacting residues is described, for example, in Lan et al., Nature volume 581, pages 215-220 (2020), the content of which is incorporated by reference it its entirety.

The coronavirus spike RBD or a portion thereof used herein can be of, or derived from, any viral species or strains in the family of Coronaviridae. The coronavirus spike RBD or a portion thereof used herein can be of any species or strains in the genus of Alpha-coronavirus. The coronavirus spike RBD or a portion thereof used herein can be of any species or strains in the genus of Beta-coronavirus. The coronavirus spike RBD or a portion thereof used herein can be of any viral species or strains in the subgenera Embecovirus (lineage A), Sarbecovirus (lineage B), Merbecovirus (lineage C), Nobecovirus (lineage D), and Hibecovirus of Beta-coronavirus.

For example, the coronavirus spike RBD protein or a portion thereof can be of any species or strains in the subgenus of Embecovirus, including but not limited to Beta-coronavirus 1 (e.g., Bovine coronavirus and human coronavirus OC43), China Rattus coronavirus HKU24, Human coronavirus HKU1, Murine coronavirus (e.g., mouse heptatitis virus), and Myodes coronavirus 2JL14. The coronavirus spike RBD protein or a portion thereof can be of any viral species or strains in the subgenus of Sarbecovirus, including but not limited to SARS-CoV, SARS-CoV2, 16BO133, Bat SARS COV Rf1, Bat coronavirus HKU3 (BtCoV HKU3), LYRa11, Bat SARS-CoV/Rp3, Bat SL-CoV YNLF_31C, Bat SL-CoV YNLF_34C, SHC014-CoV, WIV1, WIV16, Civet SARS-CoV, Rc-o319, SL-ZXC21, SL-ZC45, Pangolin SARSr-COV-GX, Pangolin SARSr-COV-GD, RshSTT182, RshSTT200, RacCS203, RmYN02, RpYN06, RaTG13, Bat CoV BtKY72, and Bat CoV BM48-31. The coronavirus spike RBD protein or a portion thereof can be of any viral species or strains in the subgenus of Merbecovirus, including but not limited to Hedgehog coronavirus 1, MERS-CoV, Pipistrellus bat coronavirus HKU5, and Tylonycteris bat coronavirus HKU4. The coronavirus spike RBD protein or a portion thereof can be of any viral species or strains in the subgenus of Nobecovirus, including but not limited to, Eidolon bat coronavirus C704, Rousettus bat coronavirus GCCDC1, and Rousettus bat coronavirus HKU9. The coronavirus spike RBD protein or a portion thereof can be of any viral species or strains in the subgenus of Hibecovirus, including but not limited to Bat Hp-Betacoronavirus Zhejiang 2013.

The coronavirus spike RBD protein or a portion thereof can be of any viral species or strain in any one of the phylogenetically clustered clades of lineage B coronavirus (Sarbecovirus). For example, the coronavirus spike RBD protein or a portion thereof can be of any species or strain in clade 1, including but not limited to SARS-CoV, WIV1, LYRa11, Rs7327, Rs4231, Rs4084, and SHC014. The coronavirus spike RBD protein or a portion thereof can be of any species or strain in clade 2, including but not limited to As6526, Yunnan 2011, Shaanxi 2011, 279-2005, Rs4237, Rs4081, Rp3, Rs4247, HKU3-8, HKU3-13, GX2013, Longquan-140, YN2013, Rf4092, ZXC21, ZC45, JL2012, HuB2013, Rf1, HeB2013, and 273-2005. The coronavirus spike RBD protein or a portion thereof can be of any species or strain in clade 1/2, including but not limited to SARS-CoV2. The coronavirus spike RBD protein or a portion thereof can be of any species or strain in clade 3, including but not limited to BM48-31.

In some embodiments, the coronavirus spike RBD protein or a portion thereof can be from a coronavirus selected from: SARS-CoV, SARS-CoV2, WIV1, LYRa11, Rs7327, Rs4231, Rs4084, SHC014, As6526, Yunnan 2011, Shaanxi 2011, 279-2005, Rs4237, Rs4081, Rp3, Rs4247, HKU3-8, HKU3-13, GX2013, Longquan-140, YN2013, Rf4092, ZXC21, ZC45, JL2012, HuB2013, Rf1, HeB2013, 273-2005, and BM48-31. In some embodiments, the coronavirus spike RBD protein or a portion thereof is from a coronavirus selected from SARS, SARS-2, WIV1, SHC014, Rf1, RmYN02, pang17, RaTG13, and Rs4081.

The coronavirus spike RBD protein or a portion or a variant thereof can, for example, comprise, or consist of, an amino acid sequence having at least 65% sequence identity to an amino acid sequence of any of the coronavirus spike protein RBDs disclosed herein (e.g., the coronavirus spike protein RBD of any one of SEQ ID NOs: 1-12 and 15-26). In some embodiments, the coronavirus spike protein RBD or a variant thereof comprises, or consists of, an amino acid sequence having, having about, having at least, having at least about, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, sequence identity to an amino acid sequence selected from SEQ ID NOs: 1-12 and 15-26. In some embodiments, the coronavirus spike protein RBD or a variant thereof comprises, or consists of, an amino acid sequence having at least, or having at least about 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a range between any two of these values, sequence identity to an amino acid sequence selected from SEQ ID NOs: 1-12 and 15-26. In some embodiments, the coronavirus spike protein RBD or a variant thereof comprises, or consists of, an amino acid sequence having, having about, having at least, or having at least about, 90%, 91%, 92%, 93%, 94%, 95%, 96%, 97%, 98%, 99%, 100%, or a range between any two of these values, sequence identity to an amino acid sequence selected from SEQ ID NOs: 1-12 and 15-26. In some embodiments, the coronavirus spike protein RBD herein described comprises or consists of an amino acid sequence selected from the group consisting of one of SEQ ID NOs: 1-12 and 15-26. In some embodiments, the amino acid sequence of the coronavirus spike protein RBD is selected from SEQ ID NOs: 1-12 and 15-26.

The coronavirus spike RBD or a variant thereof can, for example, comprise, or consist of, an amino acid sequence having, or having about, one, two, three, four, five, six, seven, eight, nine, ten, eleven, twelve, thirteen, fourteen, fifteen, sixteen, seventeen, eighteen, nineteen, twenty, twenty-two, twenty-three, twenty-four, twenty-five, twenty-six, twenty-seven, twenty-eight, twenty-nine, thirty, or a range between any two of these values, mismatch compared to an amino acid sequence of any of the coronavirus spike RBDs disclosed herein (e.g., the coronavirus spike RBDs having an amino acid sequence of any one of SEQ ID NOs: 1-12 and 15-26). In some embodiments, the coronavirus spike RBD or a variant thereof comprises, or consists of, an amino acid sequence having at most, or having at most about, one, two, three, four, five, six, seven, eight, nine, ten, eleven, twelve, thirteen, fourteen, fifteen, sixteen, seventeen, eighteen, nineteen, twenty, twenty-two, twenty-three, twenty-four, twenty-five, twenty-six, twenty-seven, twenty-eight, twenty-nine, thirty mismatches compared to an amino acid sequence selected from SEQ ID NOs: 1-12 and 15-26. In some embodiments, the mismatch(es) herein described occurs in the RBM of the coronavirus spike RBD.

The coronavirus spike RBD or a variant thereof can, for example, comprise, or consist of, an amino acid sequence having, having about, having at most, or having at most about one, two, three, four, five, six, seven, eight, nine, ten, eleven, twelve, thirteen, fourteen, fifteen, sixteen, seventeen, eighteen, nineteen, twenty, twenty-two, twenty-three, twenty-four, twenty-five, twenty-six, twenty-seven, twenty-eight, twenty-nine, thirty, or a range between any two of these values, deletions or insertions compared to an amino acid sequence of any of the coronavirus spike RBDs disclosed herein (e.g., the coronavirus spike RBDs having an amino acid sequence of any one of SEQ ID NOs: 1-12 and 15-26).

Accordingly, the coronavirus antigens in the context of the present disclosure can contain amino acid substitutions relative to the coronavirus proteins disclosed herein. Any amino acid substitution is permissible so long as the immunogenic activity of the protein is not significantly altered (e.g., at most 10%, 20%, 30%, 40% or 50% decrease relative to the coronavirus protein antigens disclosed herein) and the variants retain the desired activity. Preferred variants typically contains substitutions with one or more amino acids substituted with their functional equivalents.

### Carriers

A carrier as used herein can be generally referred to a biocompatible molecular system having the capability of incorporating and transporting molecules (e.g., therapeutic agents such as coronavirus antigens) to enhance their selectivity, bioavailability and efficiency. The carriers used in the methods, compositions, and systems herein described can be a biocompatible molecular system, either naturally occurring or synthetic, that can be functionalized or conjugated for coupling (e.g., covalently or non-covalently) to a plurality of protein antigens or immunogen polypeptides described herein. The carriers can comprise nanoparticles, nanotubes, nanowires, dendrimers, liposomes, ethosomes and aquasomes, polymersomes and niosomes, foams, hydrogels, cubosomes, quantum dots, exosomes, macrophages, and others identifiable to a person skilled in the art.

In some embodiments, the carrier used herein can be a nanosized carrier such as a nanoparticle. As used herein, the term “nanoparticle” can refer to a nanoscopic particle having a size measured in nanometers (nm). Size of the nanoparticles may be characterized by their maximal dimension. The term “maximal dimension” as used herein nanoparticle can refer to the maximal length of a straight line segment passing through the center of a nanoparticle and terminating at the periphery. In the case of nanospheres, the maximal dimension of a nanosphere corresponds to its diameter. The term “mean maximal dimension” can refer to an average or mean maximal dimension of the nanoparticles, and may be calculated by dividing the sum of the maximal dimension of each nanoparticle by the total number of nanoparticles. Accordingly, value of maximal dimension may be calculated for nanoparticles of any shape, such as nanoparticles having a regular shape such as a sphere, a hemispherical, a cube, a prism, or a diamond, or an irregular shape. The nanoparticles provided herein can need not be spherical and can comprise, for example, a shape such as a cube, cylinder, tube, block, film, and/or sheet. In some embodiments, the maximal dimension of the nanoparticles is in the range from about 1 nm to about 5000 nm, such as between about 20 nm to about 1000 nm, about 20 nm to about 500 nm, about 50 nm to about 300 nm, or about 100 nm to about 200 nm.

The nanoparticle can be, but is not limited to, any one of lipid-based nanoparticles (nanoparticles where the majority of the material that makes up their structure are lipids, e.g., liposomes or lipid vesicles), polymeric nanoparticles, inorganic nanoparticles (e.g., magnetic, ceramic and metallic nanoparticles), surfactant-based emulsions, silica nanoparticles, virus-like particles (particles primarily made up of viral structural proteins that are not infectious or have low infectivity), peptide or protein-based particles (particles where the majority of the material that makes up their structure are peptides or proteins) and/or nanoparticles that are developed using a combination of nanomaterials such as lipid-polymer hybrid nanoparticles formed by polymer cores and lipid shells or nanolipoprotein particles formed by a membrane forming lipid arranged in a membrane lipid bilayer stabilized by a scaffold protein as will be understood by a person skilled in the art.

In some embodiments, a carrier is made up of a plurality of monomeric subunits which assemble with one another through covalent and/or non-covalent forces to form the carrier. In some embodiments, the carrier described herein is a protein nanoparticle comprising a plurality of particle-forming proteins, which are the monomeric subunit proteins that form the protein nanoparticle. Protein nanoparticles can be categorized into non-viral protein nanoparticles and viral-like particles. Examples of non-viral protein nanoparticles include but are not limited to ferritins, vaults, heat-shock proteins, chaperonins, lumazine synthase, encapsulins, and bacterial microcompartments. Viral-like particles can be derived from viruses including but not limited to adenovirus, cowpea mosaic virus, cowpea chlorotic mottle virus, brome mosaic virus, broad bean mottle virus, bacteriophage lambda (e.g., bacteriophage lambda procapsid), MS2 bacteriophage, Qβ bacteriophage, P22 phage capsid, and others identifiable to a person skilled in the art.

In some embodiments, the nanoparticles described herein comprise a virus-like particle (VLP). VLP refers to a non-replicating, viral shell, derived from any of several viruses. VLPs can be naturally occurring or synthesized through the individual expression of viral structural proteins, which can then self-assemble into the virus-like structure. VLPs are generally composed of one or more viral proteins, such as particle-forming proteins referred to as capsid, coat, shell, surface and/or envelope proteins, or particle-forming polypeptides derived from these proteins. In some embodiments, VLPs can form spontaneously upon recombinant expression of the protein in an appropriate expression system. VLPs can differ in morphology, size and number of subunits. Methods for producing particular VLPs are known in the art. The presence of VLPs following recombinant expression of viral proteins can be detected using conventional techniques also known in the art, such as by electron microscopy, biophysical characterization, and the like (See e.g., Baker et al. (1991) Biophys. J. 60:1445-1456; and Hagensee et al. (1994) J. Virol. 68:4503-4505). For example, VLPs can be isolated by density gradient centrifugation and/or identified by characteristic density banding. Alternatively, cryoelectron microscopy can be performed on vitrified aqueous samples of the VLP preparation in question, and images recorded under appropriate exposure conditions. Any of a variety of VLPs known in the art can be used herein, including but not limited to, Aquifex aeolicus lumazine synthase, Thermotoga maritima encapsulin, Myxococcus xanthus encapsulin, bacteriophage Qbeta virus particle, Flock House Virus (FHV) particle, ORSAY virus particle, and infectious bursal disease virus (IBDV) particle. In some embodiments, the nanoparticle used herein can be a bacteriophage VLP, such as Ap205 VLP.

In some embodiments, the nanoparticles described herein comprise a self-assembling nanoparticle. A self-assembling nanoparticle typically refers to a ball-shape protein shell with a diameter of tens of nanometers and well-defined surface geometry that is formed by identical copies of a non-viral protein capable of automatically assembling into a nanoparticle with a similar appearance to VLPs. Examples of self-assembling nanoparticles include but are not limited to ferritin (FR) (e.g., Helicobacter pylori ferritin), which is conserved across species and forms a 24-mer, as well as B. stearothermophilus dihydrolipoyl acyltransferase (E2p), Aquifex aeolicus lumazine synthase (LuS), and Thermotoga maritima encapsulin, which all form 60-mers.

In some embodiments, the self-assembling nanoparticles comprise a plurality of particle-forming proteins of 2-keto-3-deoxy-phosphogluconate (KDPG) aldolase from the Entner-Doudoroff pathway of the hyperthermophilic bacterium Theremotoga maritima or a variant thereof. In some embodiments, mutations are introduced to the KDPG aldolase for improved particle yields, stability, and uniformity. For example, in some embodiments mutations can introduced to alter the interface between the wild-type protein trimer of KDPG aldolase. In some embodiments, the nanoparticle used herein is an i301 nanoparticle or a variant thereof. In some embodiments, the nanoparticle used herein is a mutated i301 nanoparticle (for example, mi3 nanoparticle). The self-assembling nanoparticles can form spontaneously upon recombinant expression of the protein in an appropriate expression system. Methods for nanoparticle production, detection, and characterization can be conducted using the same techniques developed for VLPs.

In some embodiments, the multivalent carriers and the related vaccine composition, methods and kits disclosed herein can employ any of a variety of known nanoparticles, their conservatively modified variants in which some amino acid residues are substituted with a functionally equivalent residue, as well as variants with substantially identical sequences (e.g., at least 90%, 95%, or 99% identical).

In some embodiments herein described, the carriers used herein are multivalent carriers. As opposed to a monovalent carrier which presents a single species of a coronavirus antigen, a multivalent nanoparticle presents a heterologous population of immunogens, comprising at least two coronavirus antigens of or derived from different coronavirus strains or species in the family of Coronaviridae including subfamilies, genera, or subgenera described herein. Accordingly, the heterologous immunogens presented on a multivalent carrier herein described have different protein sequences.

The term “present” as used herein with reference to a compound (e.g., an antigen) or functional group indicates attachment performed to maintain the chemical reactivity of the compound or functional group attached. Accordingly, a functional group presented on a carrier is able to perform under the appropriate conditions the one or more chemical reactions that chemically characterize the functional group. A compound presented on a carrier is able to perform under the appropriate conditions the one or more chemical reactions that chemically characterize the compound. For example, where the compound is, or comprises, a coronavirus antigen, the coronavirus antigen presented by a carrier maintains the complex of reactions that are associated with the immunological activity characterizing the coronavirus antigen. Accordingly, presentation of a coronavirus antigen indicates an attachment such that the immunological activity associated to the coronavirus antigen attached is maintained.

The heterologous coronavirus antigens presented on the multivalent carrier herein described can be displayed on its surface. Alternatively, the heterologous coronavirus antigens presented on the multivalent carrier herein described can be partially encapsulated or embedded such that at least an immunogenic portion of the coronavirus antigen is exposed and accessible by a host cell receptor so as to induce an immune response.

### Coupling of Coronavirus Antigens and Carriers

The coronavirus antigens can be covalently or non-covalently attached to a carrier. The terms “attach”, “attached”, “couple” and “coupled” are used interchangeably to refer to a chemical association of one entity (e.g., a chemical moiety) with another. The attachment can be direct or indirect such that for example where a first entity is directly bound to a second entity or where a first entity is bound to a second entity via one or more intermediate entity. In some embodiments, the C-terminus of a coronavirus antigens is attached to the N-terminus of a subunit forming the carrier.

In some embodiments, the attachment or coupling is covalent such that the attachment occurs in the context of the presence of a covalent bond between two entities. In some embodiments, the attachment or coupling is mediated by non-covalent interactions including but not limited to charge interactions, affinity interactions, metal coordination, hydrophobic interactions, hydrogen bonding interactions, van der Waals interactions, magnetic interactions, electrostatic interactions, dipole-dipole interactions, or combinations thereof. In some embodiments, encapsulation is a form of attachment. In some embodiments, the plurality of coronavirus antigens are conjugated to the multivalent carrier.

The carrier herein described can, for example, be functionalized with a functional group or a reactive moiety that is presented for binding with a corresponding functional group or a corresponding reactive moiety of a coronavirus antigen. Accordingly, the attachment between the coronavirus antigen and the multivalent carrier can occur through the binding between the functional group pair or reactive moiety pair. Exemplary functional group pairs or reactive moiety pairs include but are not limited to avidin (e.g., streptavidin, NeutrAvidin, CaptAvidin) and biotin pair, Strep-Tactin and Strep-tag pair, a thiol and a thiol-reactive moiety (e.g., maleimide, haloacetamide, iodoacetamid, benzylic halides and bromomethylketones) pair, and an amine and an amine-reactive moiety (e.g., active esters such as succinimidyl, tetrafluorophenyl, Carbodiimide, isothiocyanates, sulfonyl chlorides, dichlorotriazines, acryl halides, acyl azides).

In some embodiments, the coronavirus antigen can be attached to the multivalent carrier via chemical and/or photoreactive crosslinkers (e.g., crosslinking reagents) that contain two reactive groups, thereby providing a means of covalently linking the antigen and the carrier. The reactive groups in a chemical crosslinking reagent typically belong to the classes of functional groups, including succinimidyl esters, maleimides and iodoacetamides and others identifiable to a skilled person. Additional examples of crosslinking and photoactivatable reagents are described, for example, in thermofisher.com/us/en/home/references/molecular-probes-the-handbook/crosslinking-and-photoactivatable-reagents.html, the content of which is incorporated by reference.

In some embodiments, the coronavirus antigen can be attached to the multivalent carrier via a click chemistry moiety. The term “click chemistry,” as used herein, can refer to a chemical philosophy introduced by K. Barry Sharpless of The Scripps Research Institute, describing chemistry tailored to generate covalent bonds quickly and reliably by joining small units comprising reactive groups together. Click chemistry does not refer to a specific reaction, but to a concept including reactions that mimic reactions found in nature. In some embodiments, click chemistry reactions are modular, wide in scope, give high chemical yields, generate non-toxic byproducts, are stereospecific, exhibit a large thermodynamic driving force >84 kJ/mol to favor a reaction with a single reaction product, and/or can be carried out under physiological conditions. In particular, click chemistry reactions that can be carried out under physiological conditions and that do not produce toxic or otherwise harmful side products are suitable for the generation of hydrogels in situ. Reactive moieties that can partake in a click chemistry reaction are well known to those of skill in the art, and include, but are not limited to alkyne and azide, alkene and tetrazole or tetrazine, or diene and dithioester. Other suitable reactive click chemistry moieties suitable for use in the context of antigen binding are known to those of skill in the art.

In some embodiments, the coronavirus antigen is attached to the multivalent carrier (e.g., particle-forming proteins of the multivalent carrier) through a Spy tag/SpyCatcher binding pair. The Spy tag/SpyCatcher binding pair refers to a protein ligation system that is based on the internal isopeptide bond of the CnaB2 domain of FbaB from Streptococcus pyogenes (see, e.g., Zakeri et al., Proc. Natl. Acad. Sci. USA. 2012; 109:E690-E697). CnaB2 is split and engineered into two complementary fragments, such that the first fragment (SpyCatcher) is able to bind and form a covalent isopeptide bond with the second fragment (SpyTag) through the side chains of a lysine in SpyCatcher and an aspartate in SpyTag. Multivalent carriers presenting a plurality of coronavirus antigens can then be generated as a result of SpyTag/SpyCatcher mediated conjugation of the antigens to the carriers. The SpyTag/SpyCatcher binding system can in some embodiments provide improved stability and specificity of the interaction between the coronavirus antigens and the particle-forming proteins of the multivalent carrier.

In some embodiments, the particle-forming protein of the multivalent carrier is a fusion protein containing amino acid sequences from at least two unrelated proteins that have been joined together, via peptide bond, to make a single protein. For example, the coronavirus protein antigen can be fused to a SpyTag motif and the carrier subunit sequence can be fused to a SpyCatcher motif. Alternatively, the coronavirus protein antigen can be fused to a SpyCatcher motif and the carrier subunit sequence can be fused to s SpyTag motif. The coronavirus antigen of the plurality of coronavirus antigens can comprise a SpyTag at the C-terminal of the coronavirus antigen and the particle-forming protein of a plurality of particle-forming proteins comprises a SpyCatcher at the N-terminal of the particle-forming protein.

In some embodiments, the particle-forming protein can be a fusion protein containing a mi3 monomeric subunit protein at the C-terminal of the particle-forming protein and a SpyCatcher protein at the N-terminal of the particle-forming protein or a fusion protein containing a AP205-CP3 monomeric subunit protein at the C-terminal of the particle-forming protein and a SpyCatcher protein at the N-terminal of the particle forming protein such that the SpyCatcher proteins are presented or displayed for binding to the SpyTag of a coronavirus antigen.

In some embodiments, the coronavirus antigen of the plurality of coronavirus antigens comprises a coronavirus S protein RBD, and the coronavirus S protein RBD comprises a Spy tag at the C-terminal of the coronavirus S protein RBD and the particle-forming protein of a plurality of particle-forming proteins comprises a SpyCatcher at the N-terminal of the particle-forming protein.

### Multivalent Carriers

In some embodiments herein described, the multivalent carrier used herein can comprise a plurality of coronavirus antigens derived from a plurality of coronaviruses, the plurality of coronaviruses being different from one another. The plurality of coronavirus antigens can comprise at least a first coronavirus antigen of a first coronavirus and a second coronavirus antigen of a second coronavirus that is different from the first coronavirus.

One coronavirus is considered being different from another coronavirus when the two coronaviruses are from different taxonomic groups, including from different strains, species, subgenera, genera, subfamilies in the Coronaviridae family. One coronavirus is also considered being different from another coronavirus when the two coronaviruses are antigenically divergent viruses. The term “antigenically divergent coronavirus” refers to a strain of coronavirus that has a tendency to mutate or has developed mutations over time, thus changing the amino acids that are displayed to the immune system. Such mutation over time can also be referred to as “antigenic drift”.

In some embodiments, the at least first coronavirus and second coronavirus are from different genera within the Coronaviridae family. In some embodiments, the at least first coronavirus and second coronavirus are from different subgenera within the same Coronaviridae family. In some embodiments, the at least first coronavirus and second coronavirus are from different species within the same Coronaviridae family. In some embodiments, the at least first coronavirus and second coronavirus are different strains within the same Coronaviridae family.

In some embodiments, the multivalent carrier comprises a plurality of coronavirus antigens, the plurality of coronavirus antigens comprising at least two, three, four, five, six, seven, or eight heterologous coronavirus antigens, each of which is of or derived from a coronavirus different from one another.

For example, the multivalent carrier can comprise a plurality of heterologous coronavirus antigens, the plurality of coronavirus antigens comprising at least a first coronavirus antigen of a first coronavirus, a second coronavirus antigen of a second coronavirus, and a third coronavirus antigen of a third coronavirus, in which the first coronavirus, the second coronavirus and the third coronavirus are different from one another.

The multivalent carrier can comprise a plurality of heterologous coronavirus antigens, the plurality of coronavirus antigens comprising at least a first coronavirus antigen of a first coronavirus, a second coronavirus antigen of a second coronavirus, a third coronavirus antigen of a third coronavirus, and a fourth coronavirus antigen of a fourth coronavirus, in which the first coronavirus, the second coronavirus, the third coronavirus, and the four coronavirus are different from one another.

The multivalent carrier can comprise a plurality of heterologous coronavirus antigens, the plurality of coronavirus antigens comprising at least a first coronavirus antigen of a first coronavirus, a second coronavirus antigen of a second coronavirus, a third coronavirus antigen of a third coronavirus, a fourth coronavirus antigen of a fourth coronavirus, and a fifth coronavirus antigen of a fifth coronavirus, in which the first coronavirus, the second coronavirus, the third coronavirus, the four coronavirus, and the fifth coronavirus are different from one another.

The multivalent carrier can comprise a plurality of heterologous coronavirus antigens, the plurality of coronavirus antigens comprising at least a first coronavirus antigen of a first coronavirus, a second coronavirus antigen of a second coronavirus, a third coronavirus antigen of a third coronavirus, a fourth coronavirus antigen of a fourth coronavirus, a fifth coronavirus antigen of a fifth coronavirus, and a sixth coronavirus antigen of a sixth coronavirus, in which the first coronavirus, the second coronavirus, the third coronavirus, the four coronavirus, the fifth coronavirus, and the sixth coronavirus are different from one another.

The multivalent carrier can comprise a plurality of heterologous coronavirus antigens, the plurality of coronavirus antigens comprising at least a first coronavirus antigen of a first coronavirus, a second coronavirus antigen of a second coronavirus, a third coronavirus antigen of a third coronavirus, a fourth coronavirus antigen of a fourth coronavirus, a fifth coronavirus antigen of a fifth coronavirus, a sixth coronavirus antigen of a sixth coronavirus, and a seventh coronavirus antigen of a seventh coronavirus, in which the first coronavirus, the second coronavirus, the third coronavirus, the four coronavirus, the fifth coronavirus, the sixth coronavirus, and the seventh coronavirus are different from one another.

The multivalent carrier can comprise a plurality of heterologous coronavirus antigens, the plurality of coronavirus antigens comprising at least a first coronavirus antigen of a first coronavirus, a second coronavirus antigen of a second coronavirus, a third coronavirus antigen of a third coronavirus, a fourth coronavirus antigen of a fourth coronavirus, a fifth coronavirus antigen of a fifth coronavirus, a sixth coronavirus antigen of a sixth coronavirus, a seventh coronavirus antigen of a seventh coronavirus, and an eighth coronavirus antigen of an eight coronavirus, in which the first coronavirus, the second coronavirus, the third coronavirus, the four coronavirus, the fifth coronavirus, the sixth coronavirus, the seventh coronavirus, and the eighth coronavirus are different from one another.

In some embodiments, the multivalent carrier can comprise a plurality of coronavirus antigens, the plurality of coronavirus antigens comprising more than eight heterologous coronavirus antigens, each of which is of or derived from a coronavirus different from one another (i.e. coronaviruses of different taxonomic groups and/or antigenically divergent viruses).

The number of heterologous coronavirus antigens presented by a multivalent carrier can be different in different embodiments. In some embodiments, the multivalent carrier herein described can present at least 2, 3, 4, 5, 6, 7, 8, 9, 10, 11, 12, 13, 14, 15, 16, 17, 18, 19, 20, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, heterologous coronavirus antigens.

The total number of coronavirus antigens presented by a multivalent carrier can be different in different embodiments. In some embodiments, the multivalent carrier can comprise a total number of coronavirus antigens about, at least, at least about, at most, or at most about 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500, or a number or a range between any two of these values.

It should be understood that in some embodiments the total number of coronavirus antigens presented by a nanoparticle is limited by the number of particle-forming subunits that make up the nanoparticle, such as the number of particle-forming lipids in lipid-based nanoparticles and the number of particle-forming proteins in protein-based nanoparticles. For example, encapsulin proteins from Thermotoga maritima form nanoparticles having 60-mers. Therefore, encapsulin-based nanoparticles (e.g., mi3 nanoparticle and i301 nanoparticle) can present a maximum of 60 protein antigens. In some embodiments, a particle-forming subunit of a carrier can be attached with more than one coronavirus antigen.

The plurality of coronavirus antigens attached to a multivalent carrier can be of a same protein type or corresponding proteins. Coronavirus antigens of a same protein type may or may not have identical amino acid sequences, but generally share some sequence homology. For example, the coronavirus S proteins of different coronaviruses are of a same protein type or corresponding proteins. As another example, envelope proteins from different coronaviruses are considered the same protein type or corresponding proteins. In some embodiments, proteins of different coronavirus taxonomic groups having the same function are considered the same protein type or corresponding proteins. In some embodiments, coronavirus antigens of a same protein type have at least 50% sequence identity, for example at least 65%, 70%, 80%, 90%, 95%, 98%, 99%, or more sequence identity.

Alternatively, in some embodiments the plurality of coronavirus antigens attached to a multivalent carrier can comprise coronavirus proteins of different protein types. Coronavirus proteins of different protein types typically have different functions. For example, the plurality of coronavirus antigens attached to a multivalent carrier can comprise coronavirus S proteins or portions thereof as well as other coronavirus proteins such as a coronavirus N protein or a portion thereof, a coronavirus HE protein or a portion thereof, a coronavirus papain-like protease or a portion thereof, a coronavirus 3CL protease or a portion thereof, and/or a coronavirus M protein or a portion thereof.

One or more of the plurality of coronavirus antigens, or each of the plurality of coronavirus antigens, can a sequence identity of about, at least, or at least about 65%, 70%, 75%, 80%, 85%, 90%, 95%, 98%, or 99% with one another. In some embodiments, the plurality of coronavirus antigens each comprise a coronavirus S protein RBD or a portion thereof, the coronavirus S protein RBDs or portions thereof having a sequence identity of about, at least, or at least about, 65%, 70%, 75%, 80%, 85%, 90%, 95%, 98%, or 99% with one another. In some embodiments, the plurality of coronavirus antigens each comprise an amino acid sequence having at least 65%, 70%, 75%, 80%, 85%, 90%, 95%, or 98% sequence identity to an amino acid sequence selected from SEQ ID NOs: 1-12 and 15-26. In some embodiments, the plurality of coronavirus antigens each comprise an amino acid sequence selected from SEQ ID NOs: 1-12 and 15-26.

The number of attached coronavirus antigens of different coronaviruses can be the same or different. For example, the number of the first coronavirus antigens of the first coronavirus and the number of the second coronavirus antigens of the second coronavirus can be in a ratio from 1:50 to 50:1. In some embodiments, the ratio can be, be about, be at least, be at least about, be at most, be at most about, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any two of the values. In some embodiments, the ratio can be, be about, be at least, be at least about, be at most, be at most about, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any two of the values.

In some embodiments, the number of the coronavirus antigens of a coronavirus and the number of the coronavirus antigens of another coronavirus can be in a ratio from 1:50 to 50:1. In some embodiments, the ratio can be, be about, be at least, be at least about, be at most, be at most about, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any two of the values. In some embodiments, the ratio can be, be about, be at least, be at least about, be at most, be at most about, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any two of the values.

The multivalent carrier herein described can induce broadly protective anti-coronavirus responses by eliciting broadly neutralizing antibodies. Broadly neutralizing antibodies are antibodies that can neutralize coronaviruses from a taxonomic group that is not only the same as but also differs from the taxonomic groups of the coronaviruses from which the coronavirus antigens used to elicit the antibodies are derived. Broadly neutralizing response can also be referred to as heterologously neutralizing response. In some embodiments, the multivalent carriers herein described can elicit broadly neutralizing antibodies that neutralize one or more coronaviruses from a subfamily, genus, subgenus, species, and/or strain that differ from the subfamily, genus, subgenus, species, and/or strain of the coronaviruses from which the coronavirus antigens are derived to produce the multivalent carriers.

In some embodiments, the multivalent carrier comprising heterologous coronavirus antigens from a plurality of coronaviruses including a first coronavirus and a second coronavirus can induce heterologous binding and neutralizing responses against not only the first coronavirus and the second coronavirus, but also against one or more coronaviruses different from the first coronavirus and the second coronavirus (e.g., a third coronavirus, a fourth coronavirus, etc.). In particular, the multivalent carrier comprising heterologous coronavirus antigens from a plurality of coronaviruses not including one or more particular coronaviruses can induce heterologous binding and neutralizing responses against the one or more particular coronaviruses.

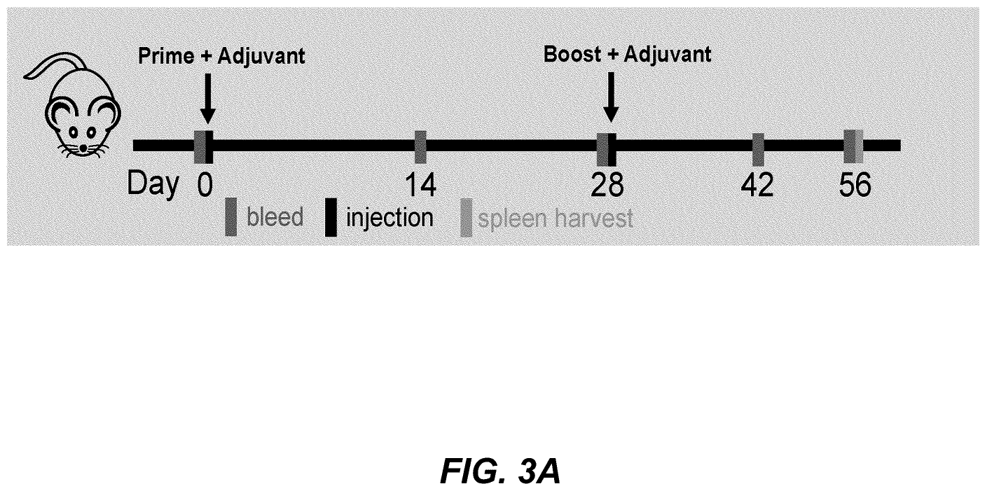

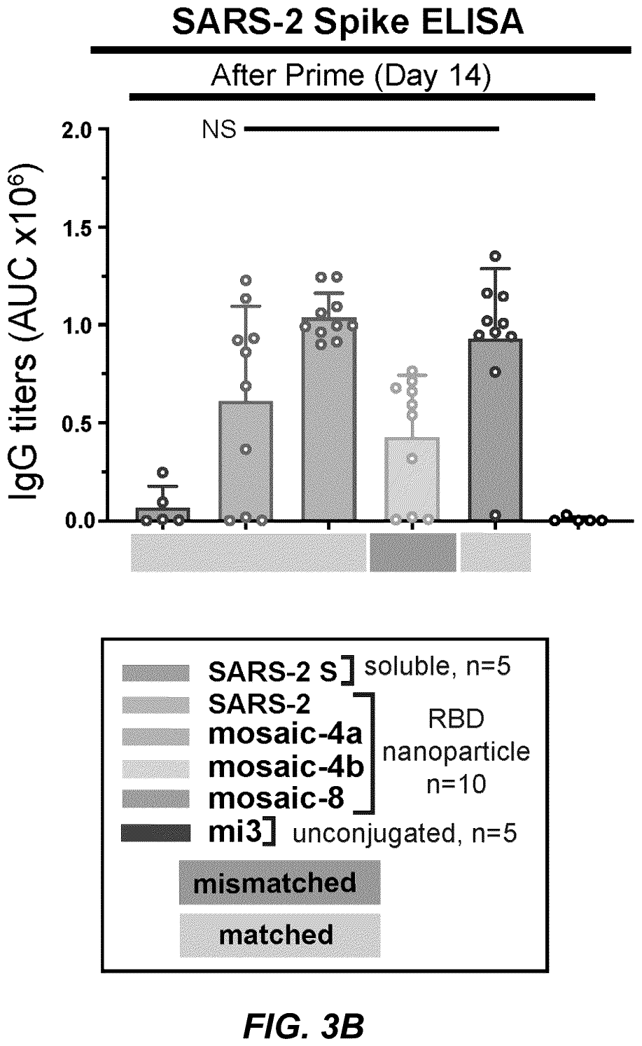

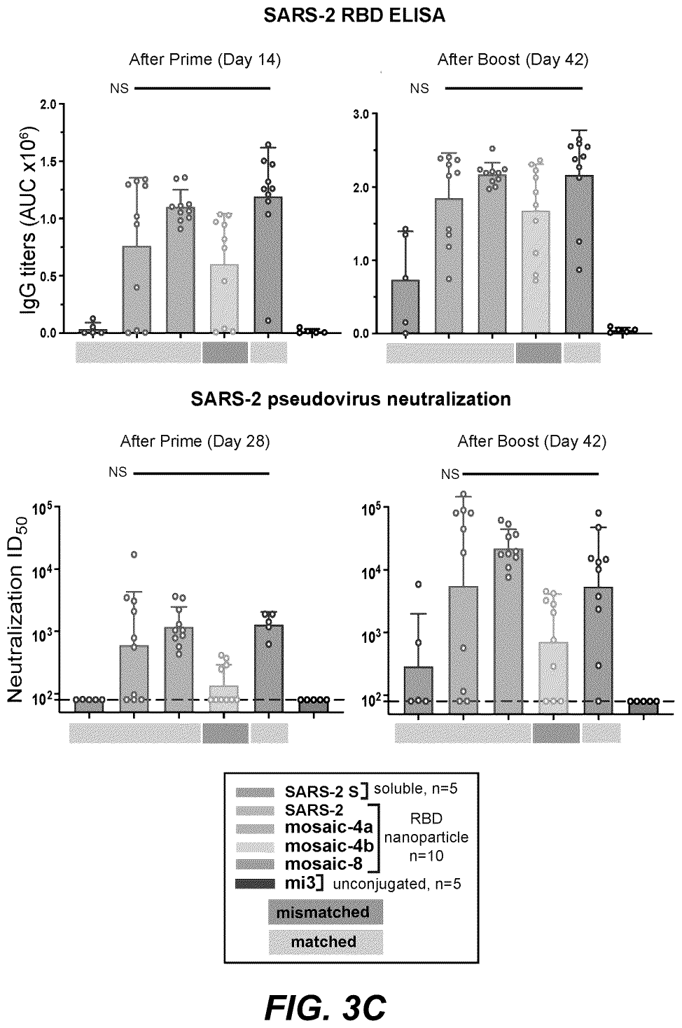

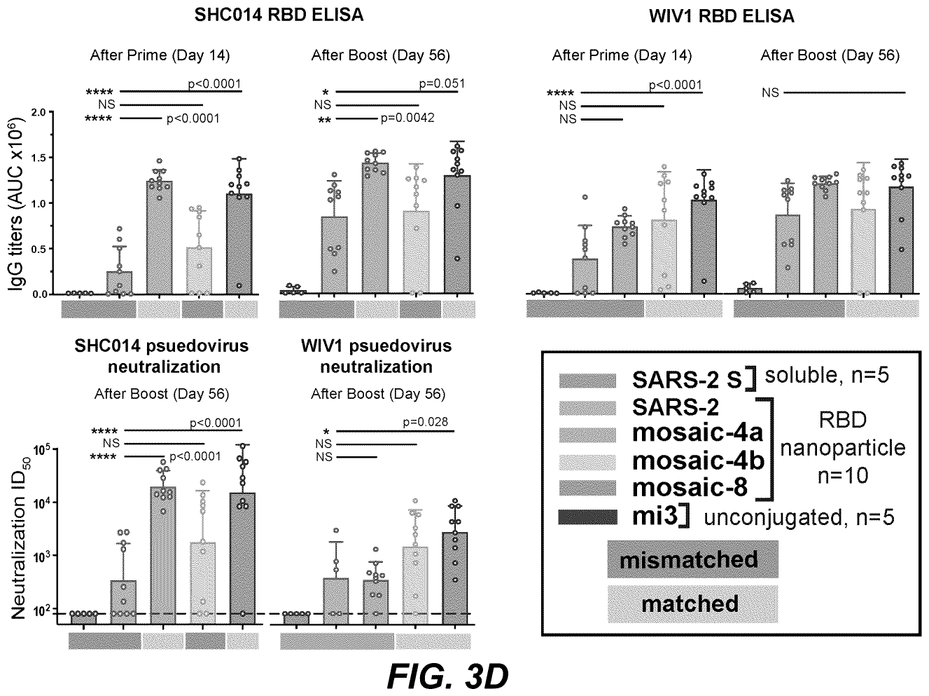

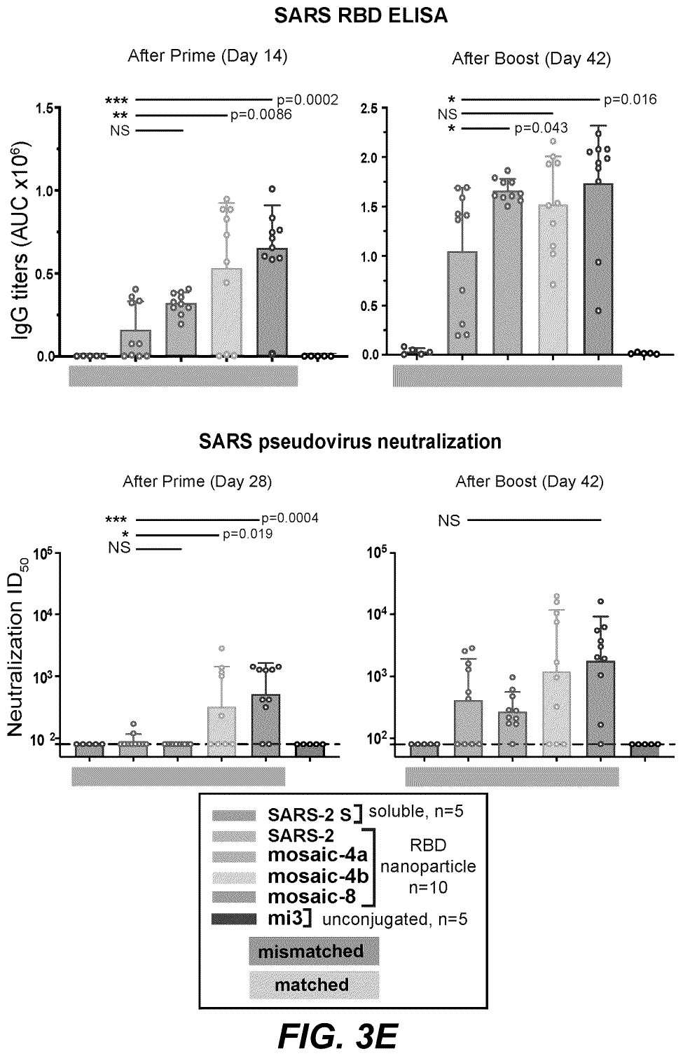

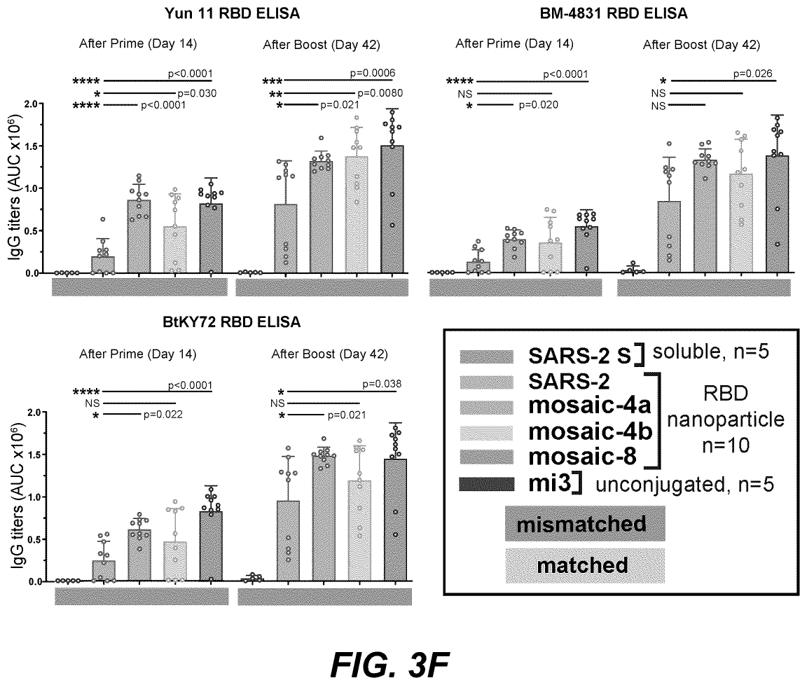

For example, if a multivalent carrier is constructed using coronavirus antigens from SARS-CoV2 and SHC014-CoV, antibodies elicited by such multivalent carrier are able to neutralize one or more coronaviruses of genera, subgenera, species and/or strains other than SARS-CoV2 and SHC014-CoV. As another example, a multivalent carrier constructed using coronavirus antigens from WIV1, Rf1, RmYN02 and pang17 can elicit heterologous binding and neutralizing responses against not only WIV1, Rf1, RmYN02 and pang17, but also other coronaviruses such as SARS-CoV2, SHC014, SARS-CoV, Yun 11, BM-4831 and BtKY72 (see, for example, FIGS. 3C-F). In yet another example, a multivalent carrier constructed using coronavirus antigens from SARS-CoV-2, SHC014, RaTG13, and Rs4081 can elicit heterologous binding and neutralizing responses against not only SARS-CoV-2, SHC014, RaTG13, and Rs4081, but also other coronaviruses such as WIV1, SARS-CoV, Yun 11, BM-4831 and BtKY72 (see, for example, FIGS. 3C-F).

In some embodiments, the multivalent carrier comprising heterologous coronavirus antigens from a plurality of coronaviruses including a first coronavirus and a second coronavirus can induce about the same or comparable magnitude (e.g., about, at least, at least about 80%, 85%, 90%, 95%, 100%, 105%, 110%, 115%, 120%, 125%, 130%, 140%, 150%, or a number or a range between any two of these values, relative to one another) of immune response against the first coronavirus and/or the second coronavirus when compared to a monovalent carrier (also referred to as a “homotypic nanoparticle”) comprising a homologous population of a single coronavirus antigen from the first coronavirus or the second coronavirus. In other words, co-display of coronavirus antigens from coronaviruses of different taxonomic groups does not diminish the immune response against a coronavirus (e.g., SARS-CoV2) relative to homotypic carriers presenting antigens from the coronavirus (e.g., homotypic SARS-CoV2 nanoparticle). In a non-limiting example, in terms of the magnitude of immune response against SARS-CoV2, it can be advantageous for conducting immunization with a mosaic nanoparticle that includes SARS-CoV2 protein antigen as well as other coronavirus protein antigens versus a homotypic SARS-CoV2 nanoparticle (see e.g., FIG. 3C).

In some embodiments, the multivalent carrier comprising heterologous coronavirus antigens from a plurality of coronaviruses including a first coronavirus and a second coronavirus can induce an increased magnitude of immune response against the first coronavirus and/or the second coronavirus when compared to a monovalent carrier comprising a homologous population of a single coronavirus antigen from the first coronavirus or the second coronavirus. The magnitude of immune response induced by the multivalent carrier can be about, at least, or at least about 0.2, 0.5, 0.9, 1.1, 1.5, 1.7, 2, 3, 4, 5, 6, 7, 8, 9, 10, fold(s), or a number or a range between any of these values, greater than by the monovalent carrier. In some embodiments, the magnitude of immune response induced by the multivalent carrier can be increased by about, at least, or at least about 5%, 10%, 20%, 30%, 50%, 75%, 100%, 110%, 120%, 150%, 200%, 250%, 300%, 400%, 500%, 600%, 700%, 800%, 900%, 1000%, or a number or a range between any of these values, as compared to that by the monovalent carrier.

In some embodiments, the multivalent carrier does not present a coronavirus antigen from a particular coronavirus, but can still produce broadly neutralizing antibodies against that particular coronavirus, for example, at a comparable or even enhanced magnitude as compared to a monovalent carrier presenting coronavirus antigens from that particular coronavirus. For example, the multivalent carrier comprising heterologous coronavirus antigens from a plurality of coronaviruses not including a first coronavirus can induce about the same or comparable magnitude (e.g., about, at least, at least about 80%, 85%, 90%, 95%, 100%, 105%, 110%, 115%, 120%, 125%, 130%, 140%, 150%, or a number or a range between any of these values, relative to one another) of immune response against the first coronavirus when compared to a monovalent carrier comprising a homologous population of a single coronavirus antigen from the first coronavirus.

In some embodiments, the multivalent carrier comprising heterologous coronavirus antigens from a plurality of coronaviruses not including a first coronavirus can elicit an enhanced heterologous binding and neutralizing response against the first coronavirus when compared to a monovalent carrier comprising a homologous population of a single coronavirus antigen from a second coronavirus. The first and second coronaviruses are different from one another. The magnitude of neutralizing response induced by the multivalent carrier can be about, at least, or at least about 0.2, 0.5, 0.9, 1.1, 1.5, 1.7, 2, 3, 4, 5, 6, 7, 8, 9, 10 fold, or a number or a range between any of these values, greater than by the monovalent carrier. In some embodiments, the magnitude of immune response induced by the multivalent carrier can be increased by about, at least, or at least about 5%, 10%, 20%, 30%, 50%, 75%, 100%, 110%, 120%, 150%, 200%, 250%, 300%, 400%, 500%, 600%, 700%, 800%, 900%, 1000%, or a number or a range between any of these values, as compared to that by the monovalent carrier.

In some embodiments, the multivalent carrier comprising heterologous coronavirus antigens from a plurality of coronaviruses including a first coronavirus and a second coronavirus can elicit a substantially enhanced neutralizing response against the first coronavirus and/or the second coronavirus when compared to a soluble coronavirus antigen from the first coronavirus or the second coronavirus. The magnitude of neutralizing response induced by the multivalent carrier can be about, at least, or at least about 1, 2, 3, 4, 5, 6, 7, 8, 9, 10, 50, 100, 200, 300, 400, 500, 600, 700, 800, 900, 1000 fold, or a number or a range between any of these values, greater than by the soluble coronavirus antigen.

The multivalent carriers herein described can be prepared using any standard molecular biology procedures known to the person skilled in the art as well as the protocols exemplified herein (see e.g., Example 2). In some embodiments, particle-forming subunits and/or the coronavirus protein antigens can be produced by liquid-phase or solid-phase chemical protein synthetic methods known to those of skill in the art.

Production of the particle-forming subunits and/or the coronavirus protein antigens can use recombinant DNA technology well known in the art. For example, a tagged coronavirus protein antigen or a coronavirus protein antigen functionalized with a protein tag can be synthesized using biosynthetic methods such as cell-based or cell-free methods known to the person skilled in the art. A tagged coronavirus protein antigen can be produced using an expression vector comprising a nucleic acid molecule encoding the coronavirus protein antigen. The nucleic acid molecule can be operably linked to appropriate regulatory elements including, but not limited to, a promoter, enhancer, transcription initiation site, termination site, and translation initiation site. The vector can also comprise a nucleic acid molecule encoding one or more protein tags (e.g., a poly(His) tag, SpyTag). In some embodiments, the vector can additionally include a nucleic acid molecule encoding a trimerization motif (e.g., a foldon trimerization domain from T4 fibritin or viral capsid protein SHP). The vector can also comprise a nucleic acid molecule encoding a signal peptide that directs the protein into the proper cellular pathway, such as a signal peptide for secretion of the expressed protein into supernatant medium. The vector may comprise one or more selectable marker genes such as gene providing ampicillin resistance or kanamycin resistance. Methods for the construction of nucleic acid constructs are well known. See, for example, Molecular Cloning: a Laboratory Manual, 3^{rd} edition, Sambrook et al. 2001 Cold Spring Harbor Laboratory Press, and Current Protocols in Molecular Biology, Ausubel et al. eds., John Wiley & Sons, 1994. Protein biosynthesis of tagged coronavirus protein antigens can be performed by providing cell-based or cell-free protein translation systems with the expression vectors encoding the tagged coronavirus protein antigens. Similarly, a tagged particle-forming protein can be produced using an expression vector comprising a nucleic acid molecule encoding a particle-forming subunit and a nucleic acid molecule encoding a protein tag (e.g., SpyCatcher). In an exemplary embodiment, the multivalent carriers are produced following the protocols described in Cohen A A et al, 2021, PLOS ONE 16(3): e0247963, the content of which is incorporated herein by reference.

In some embodiments, constructs expressing the carrier subunit and the coronavirus antigens can be introduced together into a host or transformation-competent cell. Multivalent carriers can be generated as a result of conjugation of the expressed coronavirus antigens to the self-assembled nanoparticles through a functional group pair or a reactive moiety pair described herein (e.g., SpyTag/SpyCatcher).

Carriers (e.g., nanoparticles with SpyCatcher) and coronavirus antigens (e.g., SpyTagged protein antigens) can, for example, be prepared separately and then incubated under a condition (e.g., in a TBS buffer at room temperature) for a certain time period (e.g., about, at least, or at least about 1 hour, 2 hours, 5 hours, 10 hours, 12 hours, 15 hours) to allow for the conjugation of the carriers and the coronavirus antigens. In some embodiments, the coronavirus protein antigens are provided in an excess amount as compared to the particle-forming subunits of the carriers, such as 1-fold, 2-fold, 3-fold, 4-fold, 5-fold or greater than the particle-forming subunits.

### Pharmaceutical Compositions and Therapeutic Applications

### Vaccine Compositions

Also provided herein include a vaccine composition comprising the multivalent carrier as herein described, in combination with one or more compatible and pharmaceutically acceptable carriers. A vaccine composition is a pharmaceutical composition that can elicit a prophylactic (e.g., to prevent or delay the onset of a disease, or to prevent the manifestation of clinical or subclinical symptoms thereof) or therapeutic (e.g., suppression or alleviation of symptoms) immune response in a subject.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth: (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In some embodiments, pharmaceutically acceptable carrier comprise a pharmaceutical acceptable salt. As used herein, a “pharmaceutical acceptable salt” includes a salt of an acid form of one of the components of the compositions herein described. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids.

The vaccine composition can further comprise appropriate adjuvants. Adjuvant refers to any immunomodulating substance capable of being combined with the protein antigens herein described to enhance, improve or otherwise modulate an immune response in a subject. The adjuvants can be covalently or non-covalently attached or coupled to the surface of the multivalent carrier via any of a variety approaches known in the art. Exemplary adjuvants that can be attached to the carrier include, but are not limited to, immunostimulatory peptides, oligonucleotide CpG motifs, immunostimulatory carbohydrates and polysaccharides, and immunostimulatory protein or peptide molecules (e.g. cytokines, chemokines, flagellin, and derivatives thereof), Freund's adjuvant, sapanin (e.g., Matrix M1), lecithin, aluminum hydroxide, monophosphoryl lipid A, interleukin-12, STING agonist, Advax, and AS01^{B}, STING agonist (e.g., bis-(3′,5′)-cyclic dimeric guanosine monophosphate (c-di-GMP or cdGMP)).

In some embodiments herein described, the vaccine composition comprises one or more adjuvant selected from the group comprising, or consisting of, aluminum salt-based adjuvants (e.g., aluminum hydroxide, alhydrogel), emulsion adjuvants (e.g., AddaVax™, MF59®, AS03, Freund's adjuvant, Montanide ISA51), and toll-like receptor agonists (e.g., CpG, Poly I:C, glucopyranosyl lipid A (GLA), flagellin, and resiquimod (R848)). As a skilled person will understand, both MF58® and Adda Vax™ are squalene-based oil-in-water nano-emulsion.

The vaccine composition can be formulated for a variety of modes of administration. Techniques for formulation and administration can be found, for example, in “Remington's Pharmaceutical Sciences”, 18^{th} ed., 1990, Mack Publishing Co., Easton, Pa. In some embodiments, the vaccine compositions of the present disclosure may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension: (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the hydrogel composition. The pharmaceutical compositions can comprise one or more pharmaceutically-acceptable carriers.

Formulations useful in the methods of the present disclosure include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of active ingredient which can be combined with a pharmaceutically acceptable carrier to produce a single dosage form will vary depending upon the host being treated, the particular mode of administration. The amount of active ingredient, which can be combined with a pharmaceutically acceptable carrier to produce a single dosage form will generally be that amount of the multivalent carrier which produces a therapeutic effect or an immune response. Generally, out of one hundred percent, this amount will range from about 1% to about 99% of active ingredient, preferably from about 5% to about 70%, most preferably from about 10% to about 30%.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like.

In solid dosage forms for oral administration (capsules, tablets, pills, dragees, powders, granules and the like), the multivalent carrier is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

The vaccine composition can be formulated for parenteral administration by injection, e.g. by bolus injection or continuous infusion. Formulations for injection can be presented in a unit dosage form, e.g. in ampoules or in multi-dose containers, with an optionally added preservative. The pharmaceutical compositions can further be formulated as suspensions, solutions or emulsions in oily or aqueous vehicles, and may contain other agents including suspending, stabilizing and/or dispersing agents.

### Applications

The vaccine compositions disclosed herein can be employed in a variety of therapeutic or prophylactic applications to stimulate an immune response in a subject in need, to treat or prevent a coronavirus infection in a subject in need, and/or to treat or prevent a disease or disorder caused by a coronavirus in a subject in need.

As used herein, the term “treatment” or “treat” refers to an intervention made in response to a disease, disorder or physiological condition (e.g., a coronavirus infection) manifested by a patient. The aim of treatment may include, but is not limited to, one or more of the alleviation or prevention of symptoms, slowing or stopping the progression or worsening of a disease, disorder, or condition and the remission of the disease, disorder or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. As used herein, the term “prevention” refers to any activity that reduces the burden of the individual later expressing those symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications. The term “prevent” does not require the 100% elimination of the possibility of an event. Rather, it denotes that the likelihood of the occurrence of the event has been reduced in the presence of the compound or method.

The term “condition” as used herein indicates a physical status of the body of an individual (as a whole or as one or more of its parts), that does not conform to a standard physical status associated with a state of complete physical, mental and social well-being for the individual. Conditions herein described include but are not limited disorders and diseases wherein the term “disorder” indicates a condition of the living individual that is associated to a functional abnormality of the body or of any of its parts, and the term “disease” indicates a condition of the living individual that impairs normal functioning of the body or of any of its parts and is typically manifested by distinguishing signs and symptoms.

Signs and symptoms manifesting a disease or disorder caused by a coronavirus infection can include, but not limited to, fever, cough, tiredness, a loss of taste or smell, shortness of breath or difficulty breathing, muscle aches, chills, sore throat, runny nose, headache, chest pain, pink eye (conjunctivitis), nausea, vomiting, diarrhea, rash, pneumonia and acute respiratory distress syndrome. Diseases or disorders caused by a coronavirus infection may also include severe complications including but not limited to heart disorders including arrhythmias, cardiomyopathy, acute cardiac injury, coagulation disorders including thromboembolism and pulmonary emboli, disseminated intravascular coagulation (DIC), hemorrhage, and arterial clot formation, Guillain-Barre syndrome, sepsis, shock, multiorgan failure, and multisystem inflammatory syndrome, and any combination thereof.

The terms “subject”, “subject in need”, and “individual” as used herein refer to an animal and in particular higher animals and in particular vertebrates such as mammals and more particularly human beings. In some embodiments, the subject or individual has been exposed to a coronavirus. The term “exposed” indicates the subject has come in contact with a person or an animal that is known to be infected with a coronavirus. In some embodiments, a subject in need can be a healthy subject exposed to or at risk of being exposed to a coronavirus. In some embodiments, subjects in need include those already suffering from the disease or disorder caused by a coronavirus infection or those diagnosed with a coronavirus infection.

Accordingly, the vaccine composition can be administered in advance of any symptom, for example, in advance of a coronavirus infection. The vaccine composition can also be administered at or after the onset of a symptom of disease or infection, for example, after development of a symptom of infection or after diagnosis of the infection.

The phrase “therapeutically effective amount” as used herein means that amount of multivalent carriers disclosed herein which is effective for producing some desired therapeutic effect and/or generating a desired response, such as reduce or eliminate a sign or symptom of a condition or disease, such as pneumonia, at a reasonable benefit/risk ratio. The therapeutically effective amount also varies depending on the structure and antigens of the multivalent carrier, the route of administration utilized, and the specific diseases or disorders to be treated as will be understood to a person skilled in the art. For example, if a given clinical treatment is considered effective when there is at least a 20% reduction in a measurable parameter associated with a disease or disorder, a therapeutically effective amount of the multivalent carriers for the treatment of that disease or disorder is the amount necessary to achieve at least a 20% reduction in that measurable parameter.

In some embodiments, a therapeutically effective amount is necessary to inhibit coronavirus replication or to measurably alleviate outward symptoms of the viral infection or inhibiting further development of the disease, condition, or disorder. In some embodiments, a therapeutically effective amount is an amount that prevents one or more signs or symptoms that can be caused by a coronavirus infection. In some embodiments, a therapeutically effective amount can be an amount that prevents one or more signs or symptoms of a particular disease or condition from developing, such as one or more signs or symptoms associated with coronavirus infections.

A therapeutically effective amount of the vaccine composition herein described can be estimated from data obtained from cell culture assays and further determined from data obtained in animal studies, followed up by human clinical trials. For example, toxicity and therapeutic efficacy of the vaccine compositions described herein can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices are preferred.

In some embodiments, the determination of a therapeutically effective amount of the vaccine composition can be measured by measuring the titer of antibodies produced against a coronavirus. Methods of determining antibody titers and methods of performing virus neutralization arrays are known to those skilled in the art as well as exemplified in the example section of the present disclosure (see, for example, Example 2).

In some embodiments, a method of stimulating an immune response in a subject in need is disclosed herein, the method comprising administering to the subject a pharmaceutically effective amount of the vaccine composition, thereby stimulating an immune response in the subject in need. In some embodiments, administering the vaccine composition induces neutralizing responses against coronaviruses different from the first coronavirus and the second coronavirus. In some embodiments, administering the vaccine composition induces neutralizing responses against additional coronaviruses different from the coronaviruses from which the plurality of coronavirus antigens are derived to produce the vaccine composition. In some embodiments, administering the vaccine composition induces neutralizing responses against the coronaviruses from which the plurality of coronavirus antigens are derived to produce the vaccine composition.

In some embodiments, a method for treating or preventing a coronavirus infection in a subject in need thereof is disclosed, the method comprising administering to the subject a pharmaceutically effective amount of the vaccine composition herein described, thereby treating or preventing the coronavirus infection in the subject. In some embodiments, administering the vaccine composition results in treating or preventing infection caused by a coronavirus different from the first coronavirus and the second coronavirus. In some embodiments, administering the vaccine composition results in treating or preventing infection caused by additional coronaviruses different from the coronaviruses from which the plurality of coronavirus antigens are derived to produce the vaccine composition. In some embodiments, administering the vaccine composition results in treating or preventing infection caused by the coronaviruses from which the plurality of coronavirus antigens are derived to produce the vaccine composition.

In some embodiments, a method of treating or preventing a disease or disorder caused by a coronavirus infection in a subject in need thereof is disclosed, the method comprising administering to the subject a pharmaceutically effective amount of the vaccine composition herein described, thereby treating or preventing the disease or disorder caused by the coronavirus infection in the subject. In some embodiments, administering the vaccine composition results in treating or preventing the disease or disorder caused by a coronavirus different from the first coronavirus and the second coronavirus. In some embodiments, administering the vaccine composition results in treating or preventing the disease or disorder caused by additional coronaviruses different from the coronaviruses from which the plurality of coronavirus antigens are derived to produce the vaccine composition. In some embodiments, administering the vaccine composition results in treating or preventing the disease or disorder caused by the coronaviruses from which the plurality of coronavirus antigens are derived to produce the vaccine composition.

In some embodiments, the vaccine composition can be used for treating and preventing a broad spectrum of coronavirus infections or a disease and disorder caused by such infections by inducing broadly protective anti-coronavirus responses. For example, the vaccine composition herein described can elicit broadly neutralizing antibodies that neutralize one or more coronaviruses from a subfamily, genus, subgenus, species, and/or strain that differ from the subfamily, genus, subgenus, species, and/or strain of the coronaviruses from which the coronavirus antigens are derived to produce the vaccine composition.

The vaccine compositions herein described can be administered using techniques well known to those skilled in the art, such as injection, inhalation or insulation or by oral, parenteral or rectal administration. The vaccine composition can be administered by means including, but not limited to, traditional syringes and needleless injection devices. Suitable routes of administration include, but are not limited to, parenteral delivery, such as intramuscular, intradermal, subcutaneous, intramedullary injections, as well as, intrathecal, direct intraventricular, intravenous, intraperitoneal, intranasal, or intraocular injections. For injection, the vaccine composition herein described can be formulated in aqueous solutions, preferably in physiologically compatible buffers such as Hanks' solution, Ringer's solution, or physiological saline buffer.

In some embodiments, the multivalent carriers and vaccine compositions thereof can be administered to a subject systematically. The wording “systemic administration” as used herein indicates any route of administration by which a vaccine composition is brought in contact with the body of the individual, so that the resulting composition location in the body is systemic (i.e. non limited to a specific tissue, organ or other body part where the vaccine is administered). Systemic administration includes enteral and parenteral administration. Enteral administration is a systemic route of administration where the substance is given via the digestive tract, and includes but is not limited to oral administration, administration by gastric feeding tube, administration by duodenal feeding tube, gastrostomy, enteral nutrition, and rectal administration. Parenteral administration is a systemic route of administration where the substance is given by route other than the digestive tract and includes but is not limited to intravenous administration, intra-arterial administration, intramuscular administration, subcutaneous administration, intradermal, administration, intraperitoneal administration, and intravesical infusion.

The vaccine composition herein disclosed can be administered to a subject using a prime/boost protocol. In such protocol, a first vaccine composition is administered to the subject (prime) and then after a period of time, a second vaccine composition can be administered to the subject (boost). Administration of the second composition (boost composition) can occur days, weeks or months after administration of the first composition (prime composition). For example, the boost composition can be administered about three days, 1 week, 2 weeks, 3 weeks, 4 weeks, 5 weeks, 6 weeks, 7 weeks, 8 weeks, 12 weeks, 16 weeks, 20 weeks, 24 weeks, or 28 weeks, or a number or a range between any two of these values, after the prime composition is administered. In some embodiments, the boost composition can be administered about 4 weeks after administration of the prime composition.

Therefore, the vaccine composition can be administered to the subject in need two or more times. For example, the methods herein described can comprise administering to the subject a first vaccine composition, and after a period of time, administering to the subject a second vaccine composition.

The prime vaccine composition and the boost vaccine composition can be, but need not be, the same composition. In some embodiments, the prime vaccine composition and the boost vaccine composition can contain same or different coronavirus antigens attached to the multivalent carrier. In some embodiments, the prime vaccine composition and the boost vaccine composition can contain the same coronavirus antigens attached to the multivalent carrier, but with the multivalent carrier in different pharmaceutically effective amounts. In some embodiments, the prime vaccine composition and the boost vaccine composition can contain different adjuvants. In some embodiments, the prime vaccine composition comprises the multivalent carrier disclosed herein.

The multivalent carrier and the vaccine composition thereof can be used to protect a subject against infection by heterologous coronaviruses (e.g., coronaviruses of different taxonomic groups). In other words, a vaccine composition made using coronavirus antigens of a first coronavirus and a second coronavirus is capable of protecting an individual against infection by not only the first and second coronaviruses (i.e., the matched strains), but also coronaviruses from different taxonomic groups (i.e., mismatched strains or coronavirus strains different from the first and second coronaviruses). For example, a vaccine composition made using coronavirus antigens from WIV1, Rf1, RmYN02 and pang17 can elicit broadly neutralizing antibodies, thereby protecting the subject against infection by not only WIV1, Rf1, RmYN02 and pang17 at a comparable magnitude, but also coronavirus SARS-CoV2, SHC014, SARS-CoV, Yun 11, BM-4831 and BtKY72 (see e.g., FIGS. 3C-F).

In some embodiments, the multivalent carrier and the vaccine composition thereof can protect an individual against infection by an antigenically divergent coronavirus. Therefore, in some embodiments, a vaccine composition made using coronavirus antigens of a first coronavirus and a second coronavirus is also capable of protecting an individual against infection by emerging coronavirus variants of the first and second coronaviruses. For example, a vaccine composition made using coronavirus antigens of SARS-CoV2 and SHC014 can protect an individual against infection by antigenically divergent coronavirus strains of Sarbecovirus and by diverging coronavirus strains of the future.

### Kits

The multivalent carrier and the vaccine composition containing the multivalent carrier as described herein can be provided as components of a kit.

Kits can include multivalent carriers or vaccines of the present disclosure as well components for making such multivalent carriers and vaccines. As such, kits can include, for example, primers, nucleic acid molecules, expression vectors, nucleic acid constructs encoding protein antigens and/or particle-forming subunits described herein, cells, buffers, substrates, reagents, administration means (e.g., syringes), and instructions for using any of said components. Kits can also include pre-formed carriers and coronavirus protein antigens herein described. It should be appreciated that a kit may comprise more than one container comprising any of the aforementioned, or related, components. For example, certain parts of the kit may require refrigeration, whereas other parts can be stored at room temperature. Thus, as used herein, a kit can comprise components sold in separate containers by one or more entity, with the intention that the components contained therein be used together.

### Method for Isolating Cross-Reactive Antibodies by Vaccination With Mosaic Nanoparticles

Provided herein include methods for increasing the incidence of monoclonal antibodies raised in immunization that are cross-reactive to multiple antigens. In some embodiments of the disclosed methods, multiple antigens are co-displayed randomly on a protein nanoparticle, which is used as an immunogen. Cross-reactive antibodies to multiple immunogens (both present on the nanoparticles as well as related but unmatched) can be identified from immunized animals or humans through methods provided herein, using multiple antigens as screen or bait. In some embodiments, the number of cross-reactive mAbs resulting from immunization with mosaic nanoparticles is increased compared to nanoparticles containing only a single antigen of interest. This method of generating monoclonal antibodies can be used for any combination of antigens.

### EXAMPLES

Some aspects of the embodiments discussed above are disclosed in further detail in the following examples, which are not in any way intended to limit the scope of the present disclosure.

### Example 1

### Exemplary Embodiments of Mosaic Nanoparticles Elicit Cross-Reactive Immune Responses to Zoonotic Coronaviruses in Mice

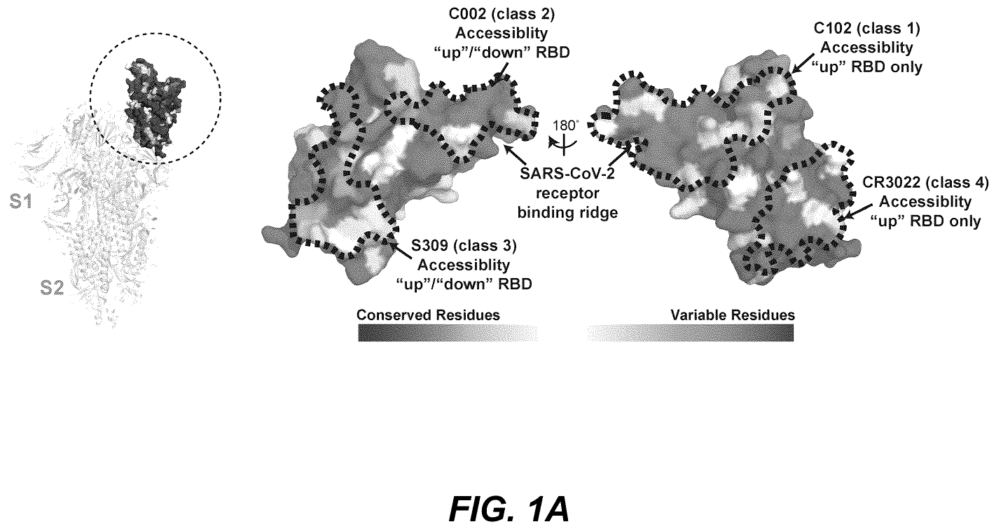

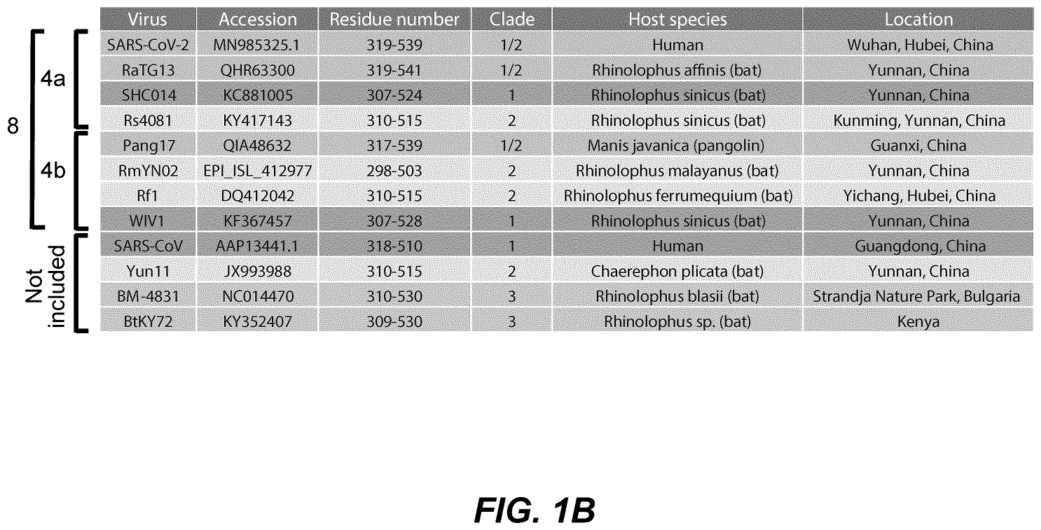

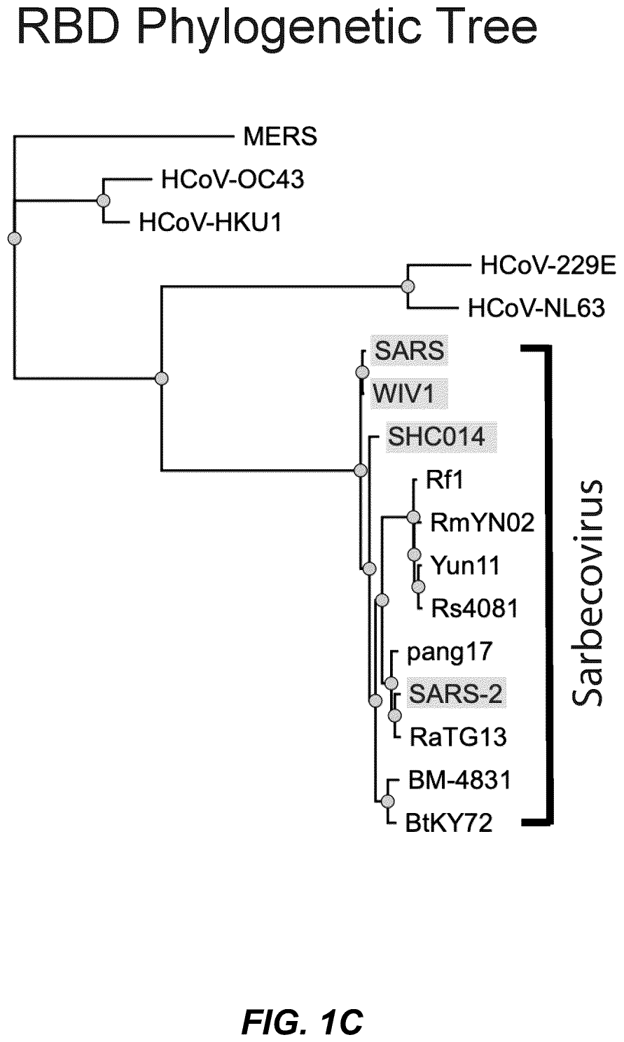

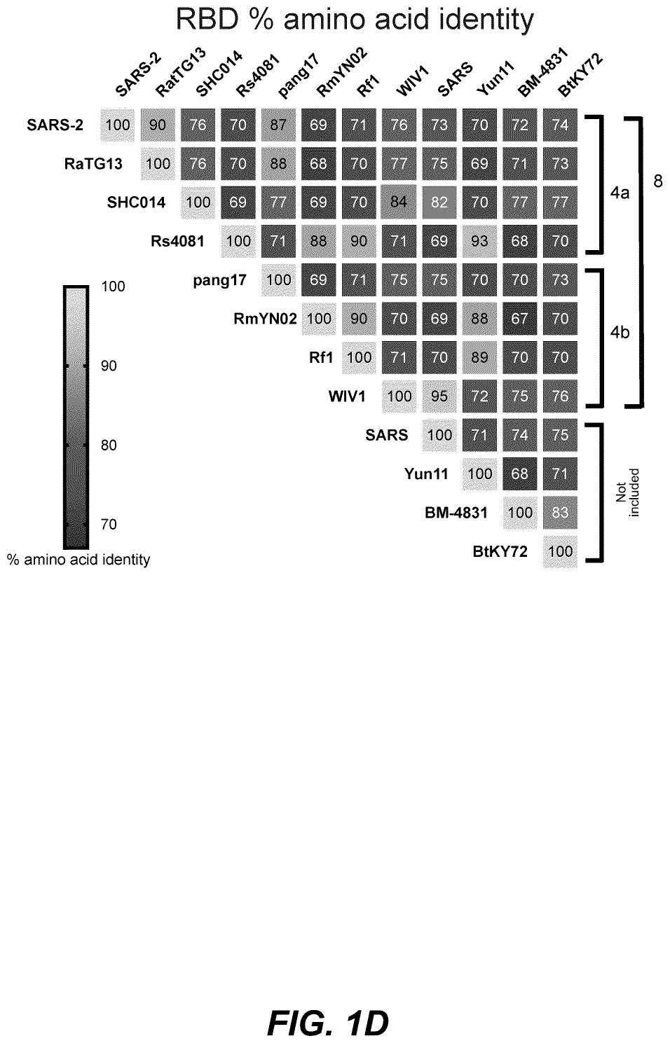

Most current SARS-CoV-2 vaccine candidates include the spike trimer (S), the viral protein that mediates target cell entry after one or more of its RBDs adopt an “up” position to bind a host receptor (FIG. 1A). The RBDs of human coronaviruses SARS-CoV-2, SARS-CoV, HCoV-NL63, and related animal coronaviruses (WIV1 and SCH014) use angiotensin-converting enzyme 2 (ACE2) as their host receptor, while other coronaviruses use receptors such as dipeptidyl peptidase 4 or sialic acids. Consistent with its function in viral entry, S is the primary target of neutralizing antibodies, with many targeting the RBD.

Without being bound by any particular theory, multivalent display of antigen enhances B-cell responses and can provide longer-lasting immunity than monovalent antigens, thus protein-based vaccine candidates often involve a nanoparticle that enables antigen multimerization. Many nanoparticles and coupling strategies have been explored for vaccine design, with “plug and display” strategies being especially useful. In one such approach, multiple copies of an engineered protein domain called SpyCatcher fused to subunits of a virus-like particle form spontaneous isopeptide bonds to purified antigens tagged with a 13-residue SpyTag. The SpyCatcher-SpyTag system was used to prepare multimerized SARS-CoV-2 RBD or S trimer that elicited high titers of neutralizing antibodies. Although promising for protection against SARS-CoV-2, coronavirus reservoirs in bats suggest future cross-species transmission, necessitating a vaccine that protects against emerging coronaviruses as well as SARS-CoV-2. Here SpyCatcher003-mi3 nanoparticles were prepared simultaneously displaying SpyTagged RBDs from human and animal coronaviruses to evaluate whether mosaic particles can elicit cross-reactive antibody responses, as previously demonstrated for influenza head domain mosaic particles. Mice immunized with homotypic or mosaic nanoparticles produced broad binding and neutralizing responses, in contrast to plasma antibodies elicited in humans by SARS-CoV-2 infection. Moreover, mosaic nanoparticles showed enhanced heterologous binding and neutralization properties against human and bat SARS-like Betacoronaviruses (Sarbecoviruses) compared with homotypic SARS-CoV-2 nanoparticles.

Protection against SARS-CoV-2 and SARS-related emergent zoonotic coronaviruses is urgently needed. In this example, homotypic nanoparticles displaying the RBD of SARS-CoV-2 or co-displaying SARS-CoV-2 RBD were made along with RBDs from animal Betacoronaviruses that represent threats to humans (mosaic nanoparticles; 4-8 distinct RBDs). Mice immunized with RBD-nanoparticles, but not soluble antigen, elicited cross-reactive binding and neutralization responses. Mosaic-RBD-nanoparticles elicited antibodies with superior cross-reactive recognition of heterologous RBDs compared to sera from immunizations with homotypic SARS-CoV-2-RBD-nanoparticles or COVID-19 convalescent human plasmas. Moreover, sera from mosaic-RBD-immunized mice neutralized heterologous pseudotyped coronaviruses equivalently or better after priming than sera from homotypic SARS-CoV-2-RBD-nanoparticle immunizations, demonstrating no immunogenicity loss against particular RBDs resulting from co-display. As shown in this example, a single immunization with mosaic-RBD-nanoparticles provides a potential strategy to simultaneously protect against SARS-CoV-2 and emerging zoonotic coronaviruses.

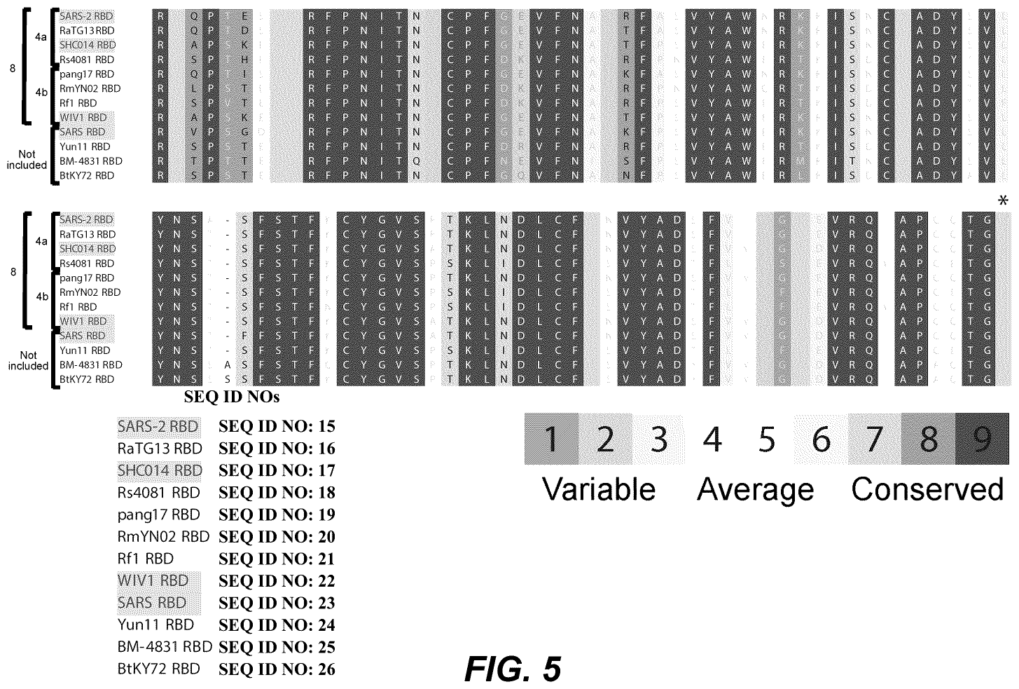

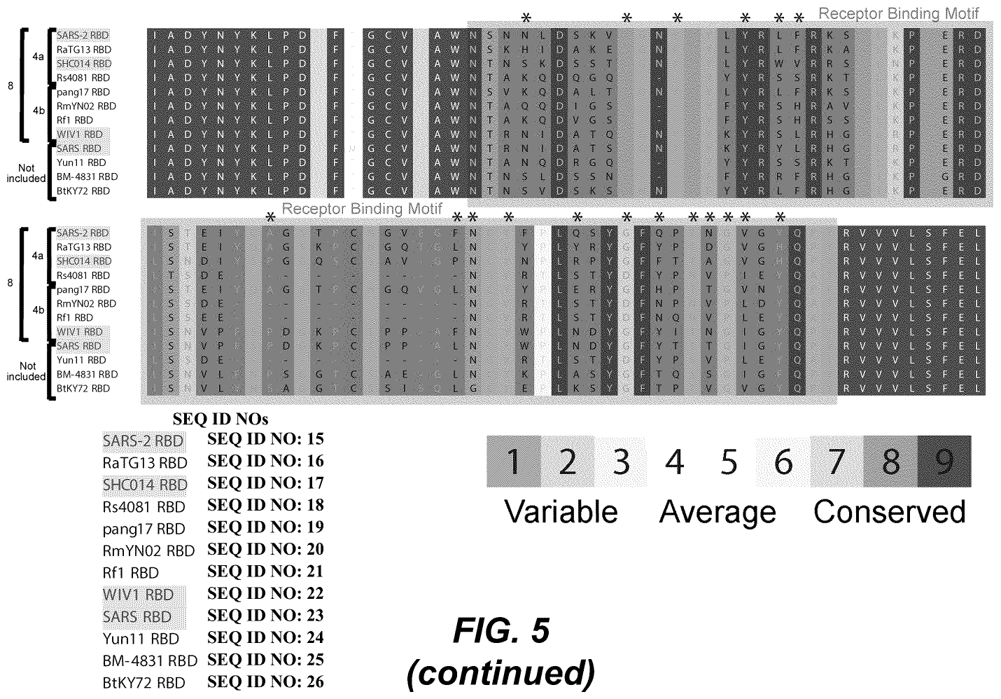

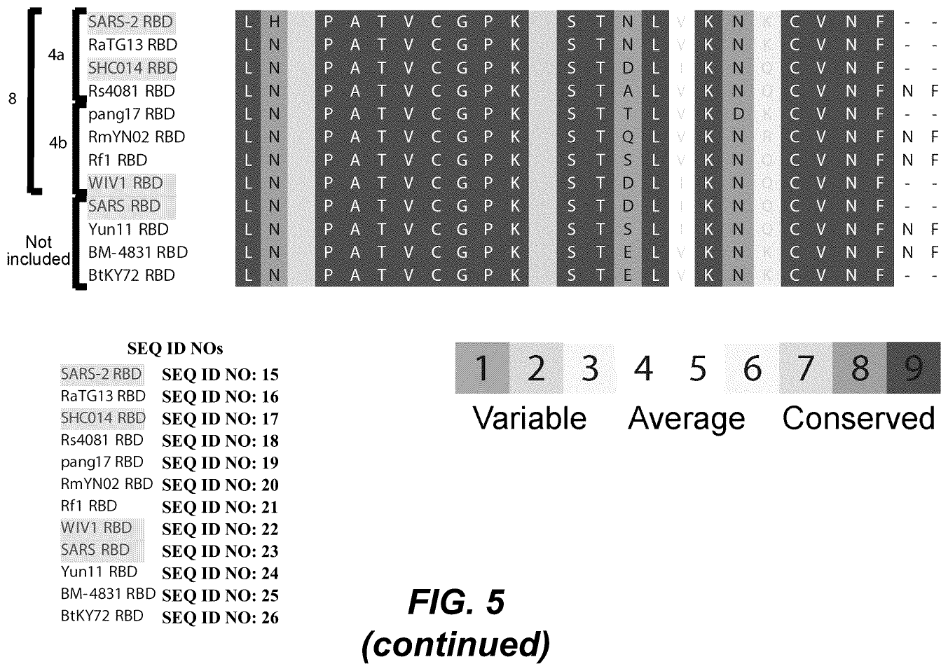

A study of Sarbecovirus RBD receptor usage and cell tropism described in Letko M et al., Nature Microbiology 5, 562-569 (2020) was used to guide the choice of RBDs for co-display on mosaic particles. From 29 RBDs that were classified into distinct clades (clades 1, 2, 1/2, and 3), diverse RBDs were identified from SARS-CoV, WIV1, and SHC014 (clade 1), SARS-CoV-2 (clade 1/2), Rs4081, Yunnan 2011 (Yun11), and Rf1 (clade 2), and BM48-31 (clade 3), of which SARS-CoV-2 and SARS-CoV are human coronaviruses and the rest are bat viruses originating in China or Bulgaria (BM48-31). Also included were RBDs from the GX pangolin clade 1/2 coronavirus (referred to here as pang17), RaTG13, the bat clade 1/2 virus most closely related to SARS-CoV-2, RmYN02, a clade 2 bat virus from China, and BtKY72, a Kenyan bat clade 3 virus. Mapping of the sequence conservation across selected RBDs showed varying degrees of sequence identity (68-95%), with highest sequence variability in residues corresponding to the SARS-CoV-2 ACE2 receptor-binding motif (FIG. 1A-FIG. 1D; FIG. 5A-FIG. 5C). 8 of the 12 RBDs were chosen for making three types of mosaic nanoparticles: mosaic-4a (coupled to SARS-2, RaTG13, SHC014, and Rs4081 RBDs), mosaic-4b (coupled to pang17, RmYN02, RF1, and WIV1 RBDs), and mosaic-8 (coupled to all eight RBDs), and they were compared with homotypic mi3 particles constructed from SARS-CoV-2 RBD alone (homotypic SARS-2). RBDs from SARS, Yun11, BM-4831, and BtKY72, which were not coupled to mosaic particles, were used to evaluate sera for cross-reactive responses.

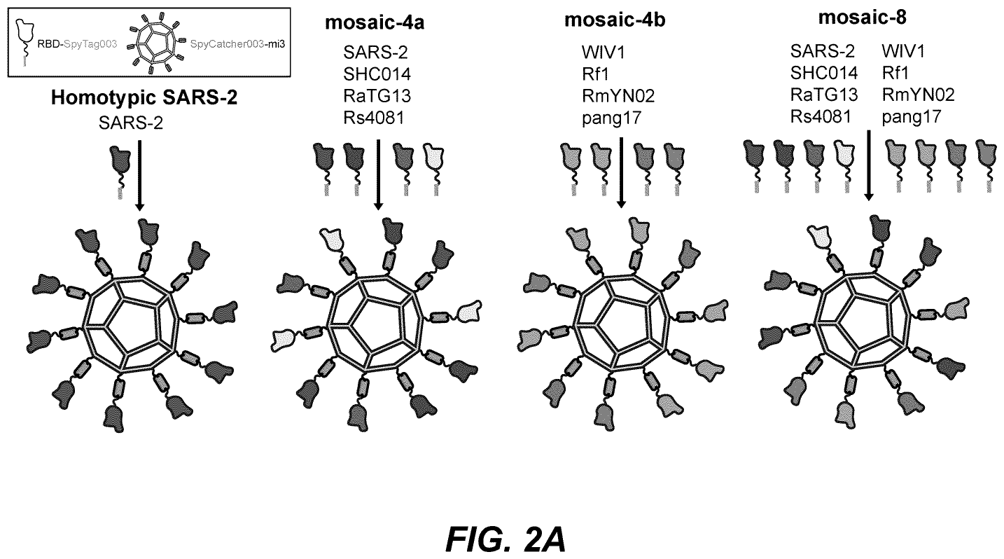

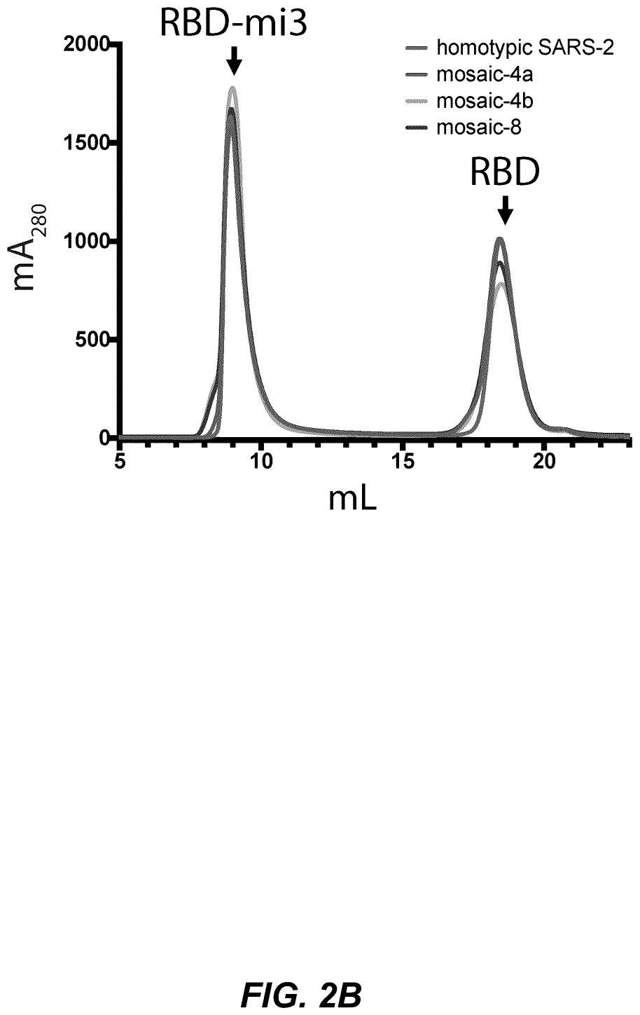

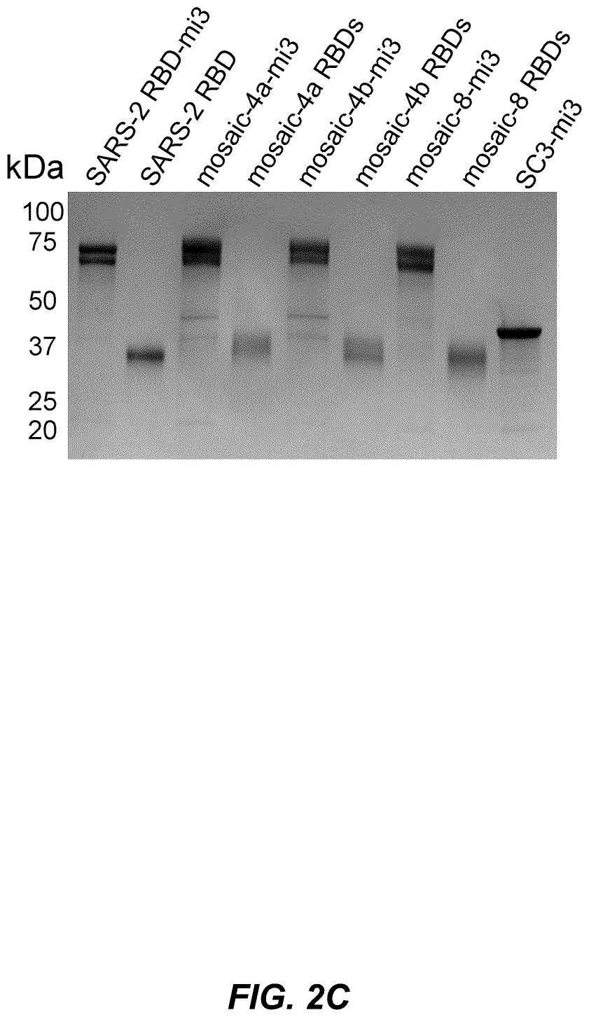

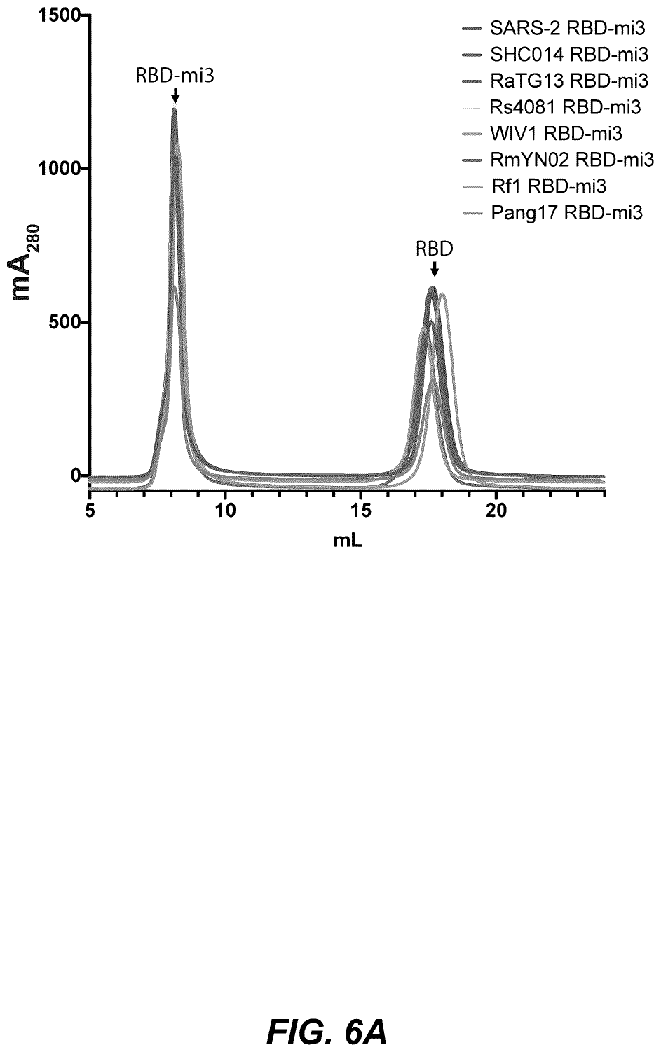

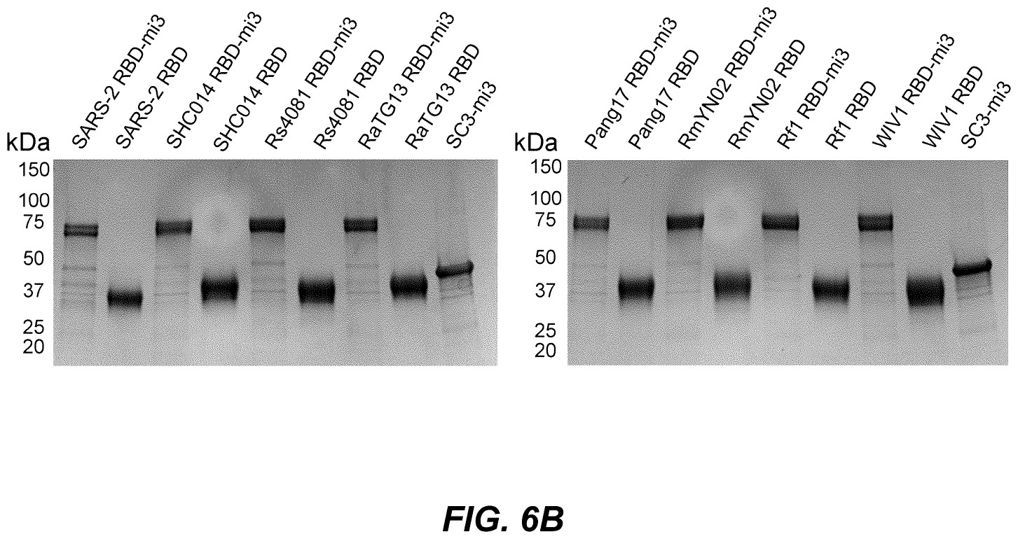

SpyTag003-RBDs were coupled to SpyCatcher003-mi3 (60 potential conjugation sites) to make homotypic and mosaic nanoparticles (FIG. 2A). Particles were purified by size exclusion chromatography (SEC) and analyzed by SDS-PAGE, revealing monodisperse SEC profiles and nearly 100% conjugation (FIG. 2B-FIG. 2C). Representative RBDs were conjugated to SpyCatcher003-mi3 with similar or identical efficiencies (FIG. 6A-FIG. 6B), suggesting that mosaic particles contained approximately equimolar mixtures of different RBDs.

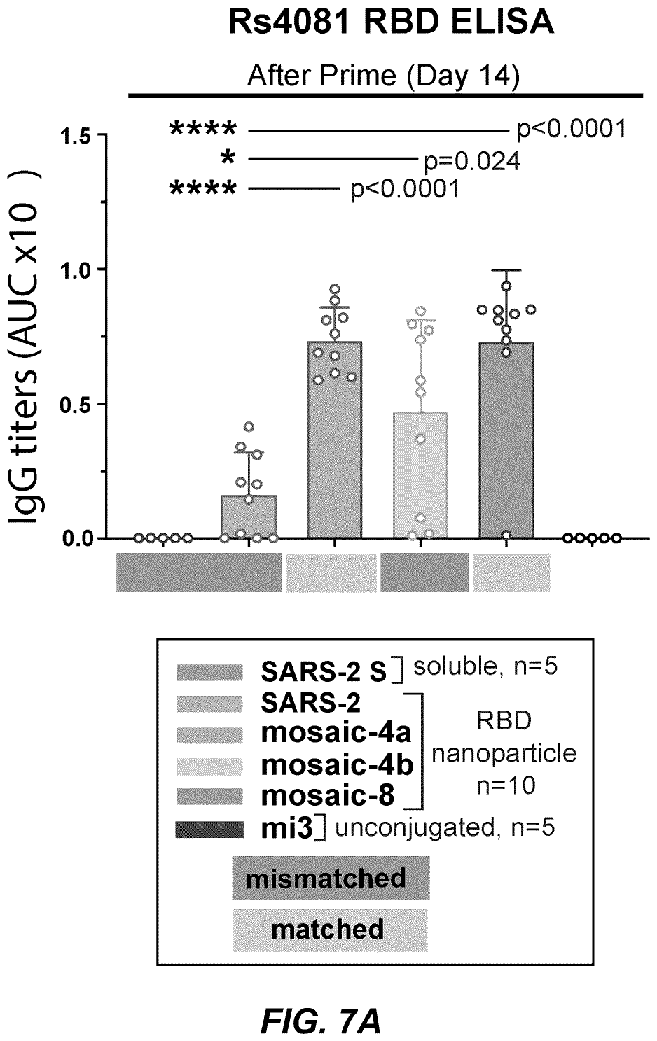

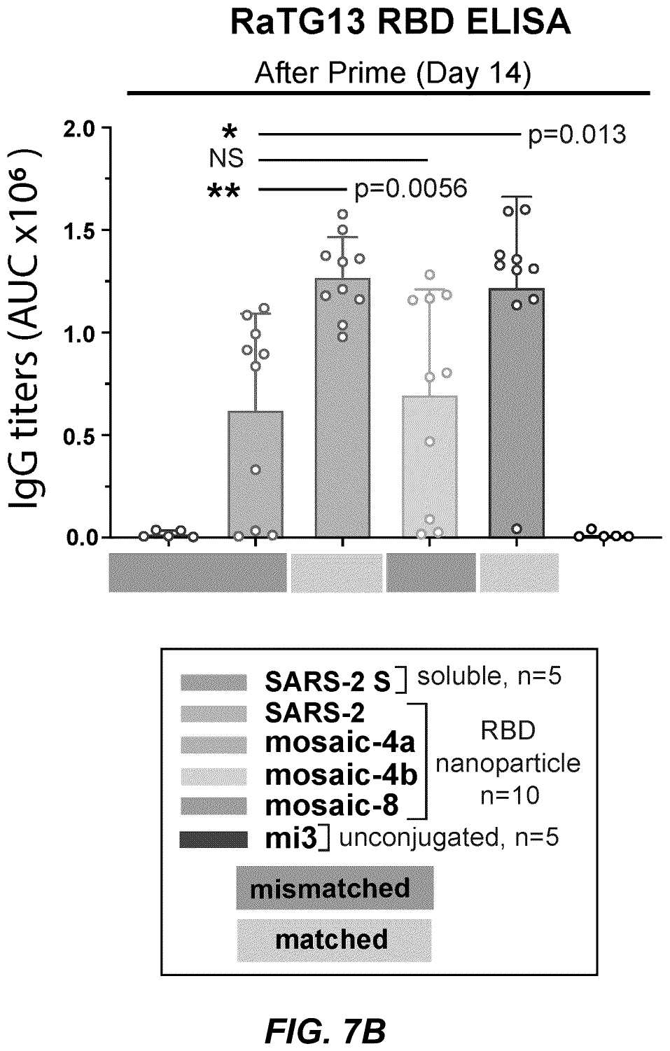

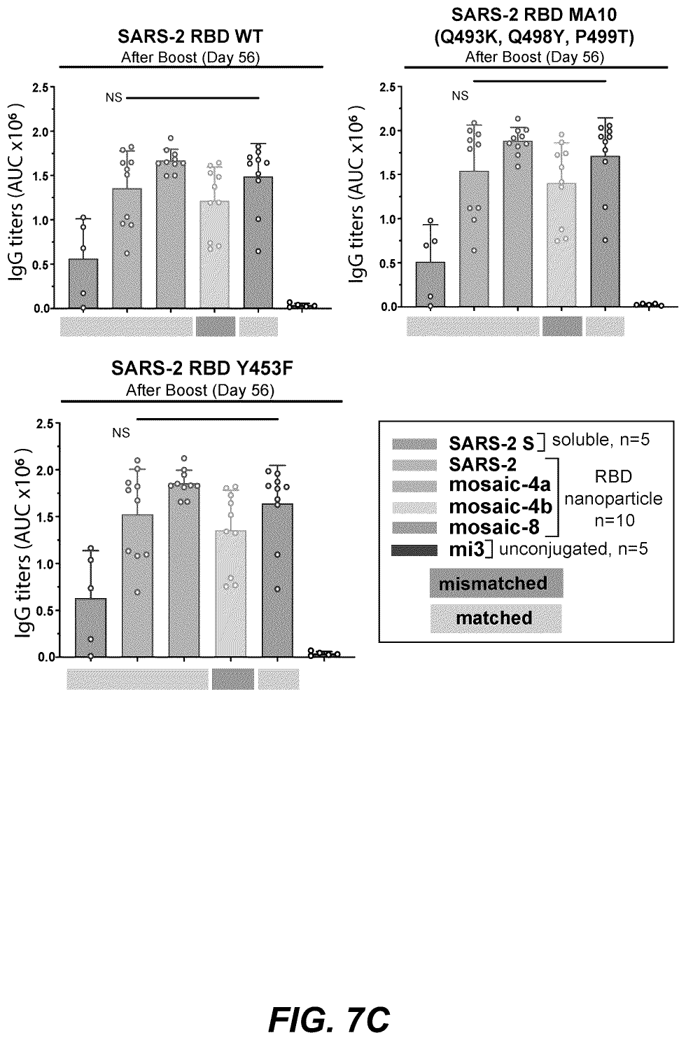

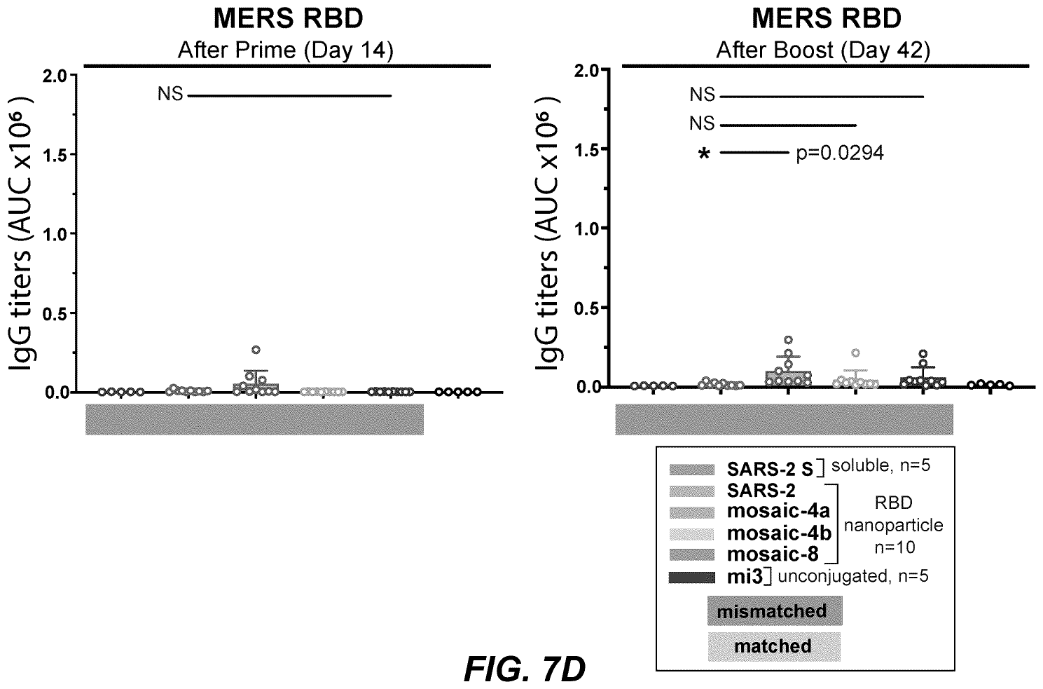

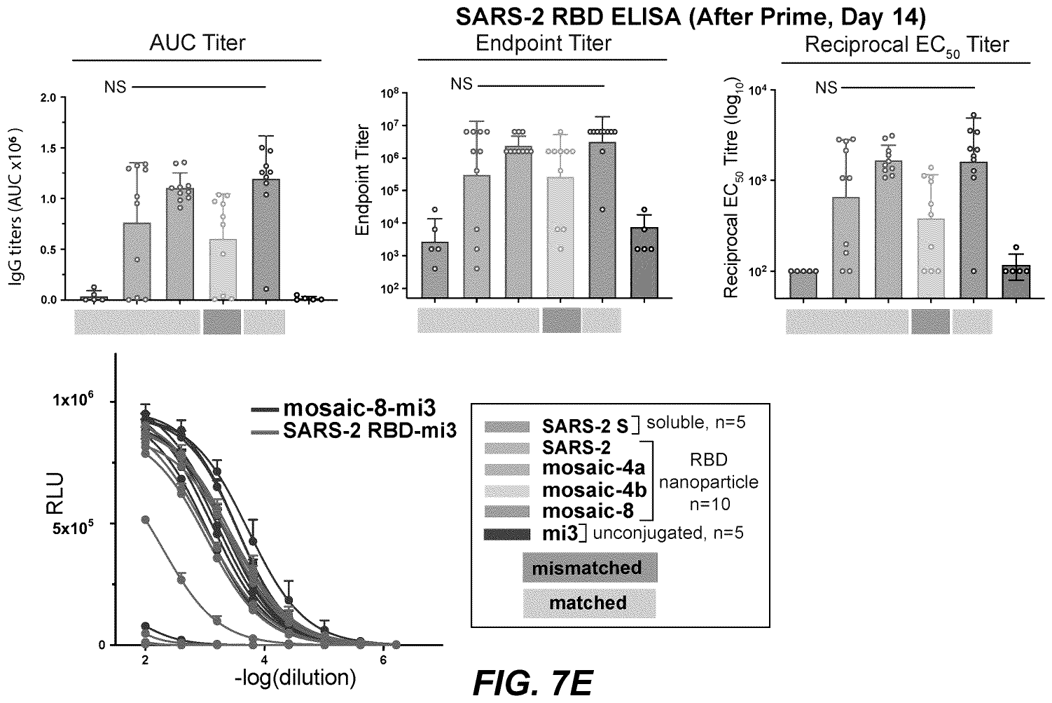

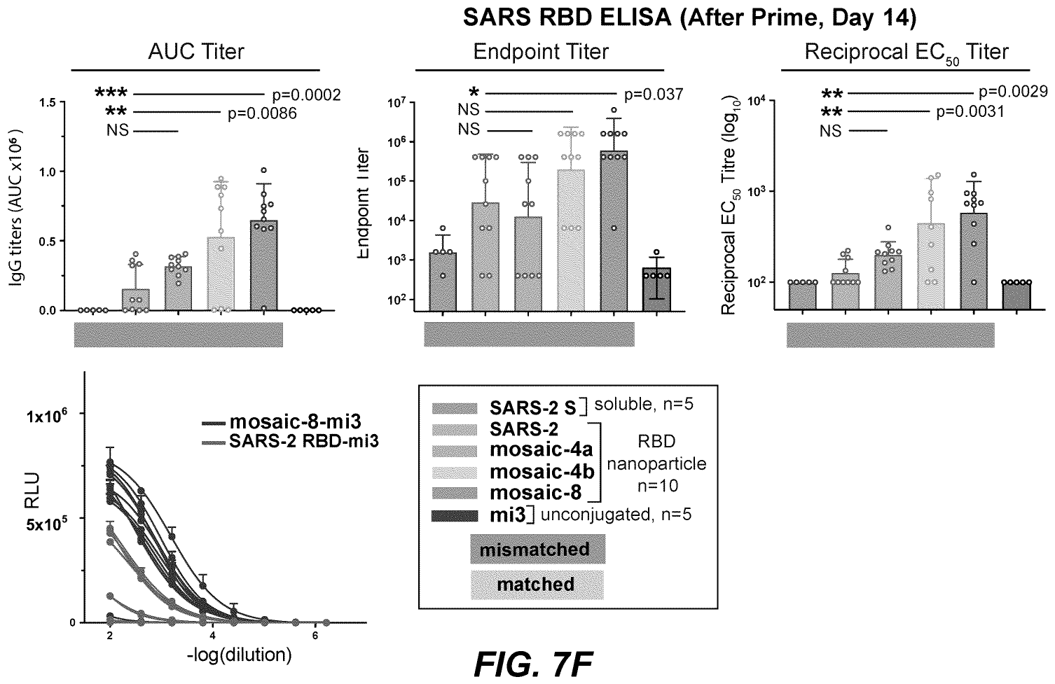

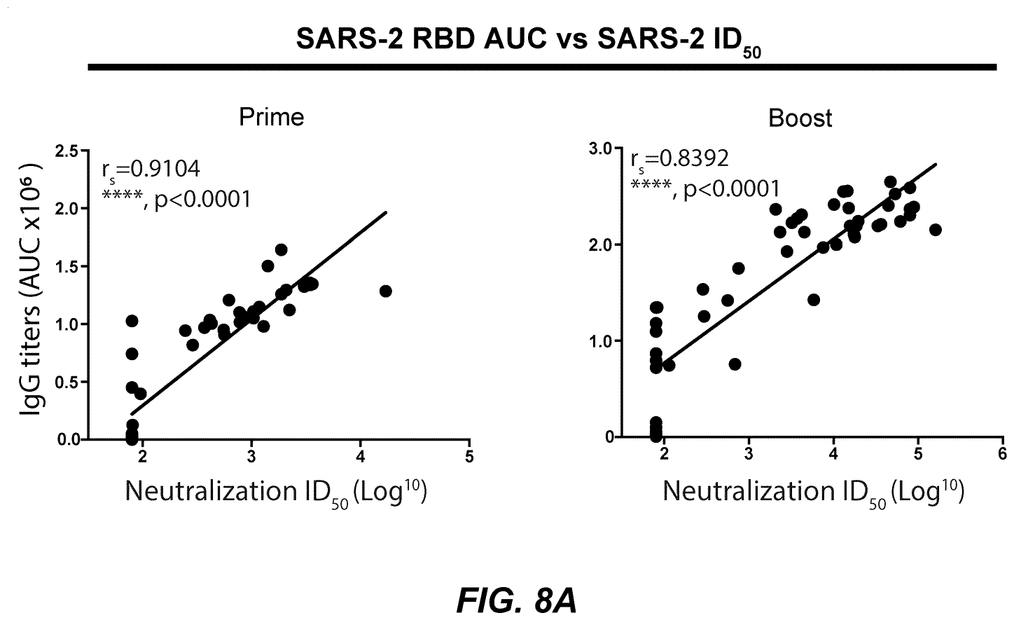

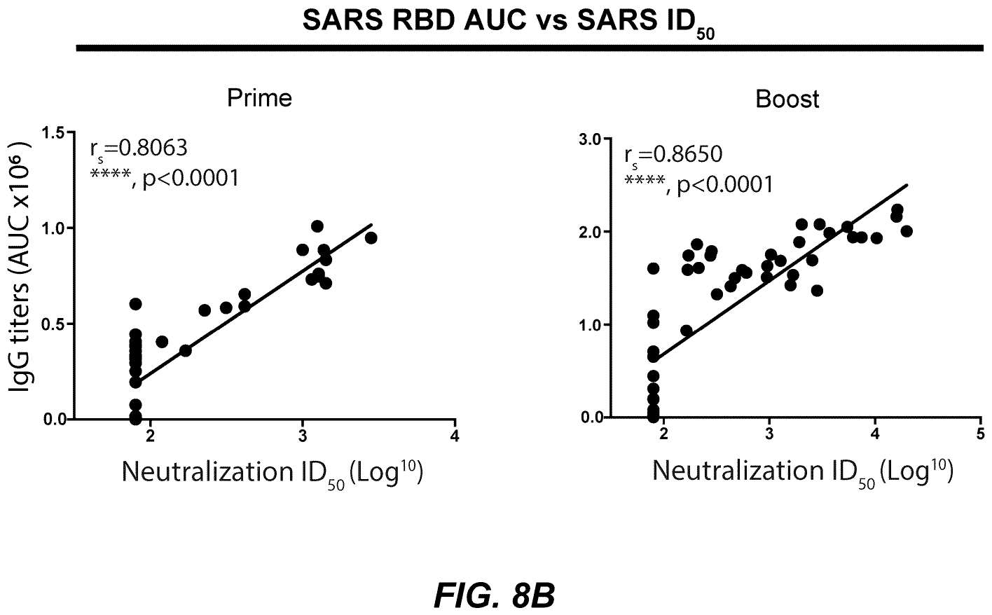

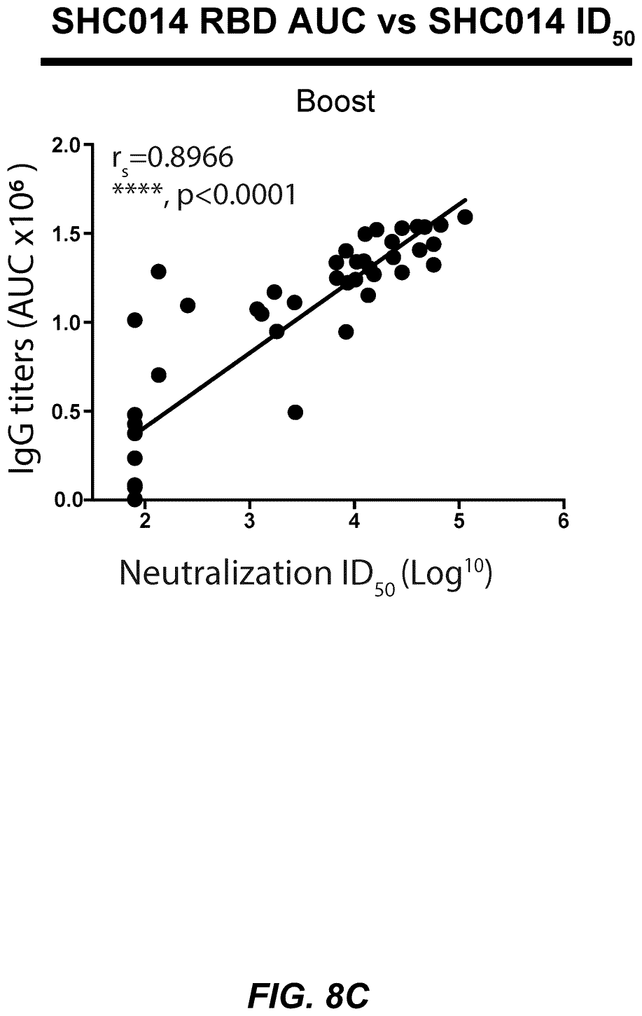

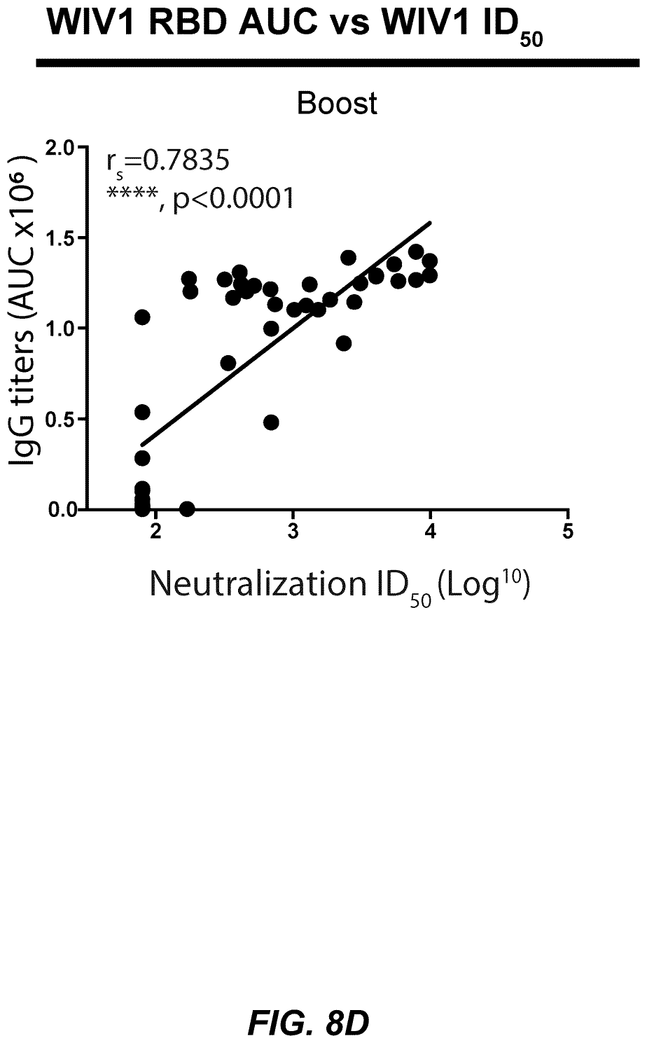

Mice were immunized with either soluble SARS-CoV-2 spike trimer (SARS-2 S), nanoparticles displaying only SARS-2 RBD (homotypic SARS-2), nanoparticles co-displaying RBDs (mosaic-4a, mosaic-4b, mosaic-8), or unconjugated nanoparticles (mi3). IgG responses were evaluated after prime or boost immunizations (FIG. 3A) by ELISA against SARS-2 S (FIG. 3B) or a panel of RBDs (FIG. 3C-FIG. 3F; FIG. 7A-FIG. 7F). Sera from unconjugated nanoparticle-immunized animals (black in FIG. 3A-FIG. 3F, FIG. 7A-FIG. 7F) showed no responses above background. Anti-SARS-2 S trimer and anti-SARS-2 RBD serum responses were similar (FIG. 3B-FIG. 3C), demonstrating that antibodies elicited against RBDs can access their epitopes on SARS-2 S trimer. In vitro neutralization assays were also conducted using a pseudotyped virus assay that quantitatively correlates with authentic virus neutralization for strains known to infect 293T_{ACE2} target cells (SARS-CoV-2, SARS, WIV1 and SHC104). Neutralization and ELISA titers were significantly correlated (FIG. 8A-FIG. 8D), thus suggesting ELISAs are predictive of neutralization results when pseudotyped neutralization assays were not possible due to unknown viral entry receptor usage.

Mice immunized with soluble SARS-2 S trimer (brown bars) showed no binding or neutralization except for autologous responses against SARS-2 after boosting (FIG. 3C-FIG. 3F). By contrast, sera from RBD-nanoparticle-immunized animals (red, green, yellow, and blue bars) exhibited binding to all RBDs (FIG. 3C-FIG. 3F; FIG. 7A) and neutralization against all four strains after boosting (FIG. 3C-FIG. 3E), consistent with increased immunogenicities of multimerized antigen on nanoparticles versus soluble antigen. Homotypic SARS-2 nanoparticles, but not soluble SARS-2 trimer, induced heterologous responses to zoonotic RBDs and neutralization of heterologous coronaviruses (FIG. 3D-FIG. 3F). To address whether co-display of SARS-2 RBD along with other RBDs on mosaic-4a and mosaic-8 versus homotypic display of SARS-2 RBD (homotypic SARS-2) diminished anti-SARS-2 responses, SARS-2-specific ELISA and neutralization titers for mosaic versus homotypic immunizations (FIG. 3C) were compared: there were no significant differences in IgG anti-SARS-2 titers for animals immunized with homotypic (red in FIG. 3C) versus mosaic nanoparticles (green and blue in FIG. 3C). Thus there was no advantage of immunization with a homotypic RBD-nanoparticle versus a mosaic-nanoparticle that included SARS-2 RBD in terms of the magnitude of immune responses against SARS-2.

Serum responses against matched RBDs (RBDs present on an injected nanoparticle; gray horizontal shading) versus mismatched RBDs (RBDs not present on injected nanoparticle; red horizontal shading) were compared next (FIG. 3A-FIG. 3F; FIG. 7A-FIG. 7F). Although SARS-2 RBD was not presented on mosaic-4b, antibody titers elicited by mosaic-4b immunization (yellow) were not significantly different than titers elicited by matched nanoparticle immunizations (homotypic SARS-2 (red), mosaic-4a (green), and mosaic-8 (blue)), and sera from boosted mosaic-4b-immunized mice neutralized SARS-2 pseudovirus (FIG. 3C). In other matched versus mismatched comparisons, sera showed binding and neutralization of SHC014 and WIV1 regardless of whether these RBDs were included on the injected nanoparticle (FIG. 3D), underscoring sharing of common epitopes among RBDs (FIG. 1A).

Demonstrating advantages of mosaic versus homotypic SARS-2 nanoparticles, sera from mosaic-8-immunized mice bound SHC014 and WIV1 RBDs significantly better after the prime than sera from homotypic SARS-2-immunized mice and retained better binding to SHC014 RBD after boosting (FIG. 3D). Thus the potential increased avidity of the homotypic SARS-2 nanoparticle displaying only one type of RBD over the mosaic-8 nanoparticles did not confer increased breadth. Moreover, mosaic-8-immunized and boosted sera were 7-44-fold more potent than sera from homotypic SARS-2-immunized animals in neutralizing SHC014 and WIV1 (FIG. 3D). Neutralization of the SHC014 and WIV1 pseudoviruses by mosaic-8 sera suggests that combining RBDs on a mosaic nanoparticle does not diminish the immune response against a particular RBD, also suggested by ELISA binding of sera to Rs4081 and RaTG13 (FIG. 7A-FIG. 7B).

To further address whether RBD-nanoparticles elicited antibodies that recognized totally mismatched strains and SARS-CoV-2 RBD mutants, sera were evaluated for binding to SARS, Yun11, BM-4831, and BtKY72 RBDs (FIG. 3E-FIG. 3F), SARS-2 RBD mutants (FIG. 7C), MERS-CoV RBD (FIG. 7D), and for neutralization in SARS pseudovirus assays (FIG. 3E). No reductions in SARS-2 RBD binding were found as a result of mutations (Y453F, the “Danish mink variant” or a Q493K/Q498Y/P499T triple mutant) (FIG. 7C), no binding of any elicited sera to MERS-CoV RBD (FIG. 7D), and higher and more cross-reactive antibody responses for mosaic immunizations compared with homotypic SARS-2 immunizations: e.g., mosaic-8-primed and boosted animals showed significantly higher titers against SARS RBD than sera from homotypic SARS-2-immunized mice (FIG. 3E). After the prime, sera from the homotypic SARS-2-immunized animals did not neutralize SARS, whereas the mosaic-4b and mosaic-8 sera were neutralizing (FIG. 3E), perhaps facilitated by these nanoparticles including WIV1 RBD, which is related by 95% amino acid identity to SARS RBD (FIG. 1D). After boosting, SARS-2 and mosaic-4a sera were also neutralizing, although titers were ˜4-fold lower than for mosaic-8-immunized animals (FIG. 3E). ELISA titers against other mismatched RBDs (Yun11, BM-4831, BtKY72) were significantly higher for sera collected after mosaic-8 priming compared to sera from homotypic SARS-2 priming, and heightened binding was retained after boosting (FIG. 3F). Thus mosaic nanoparticles, particularly mosaic-8, induce higher antibody titers against mismatched RBDs than homotypic SARS-2 nanoparticles, again favoring the co-display approach for inducing broader anti-coronavirus responses, especially after a single prime.

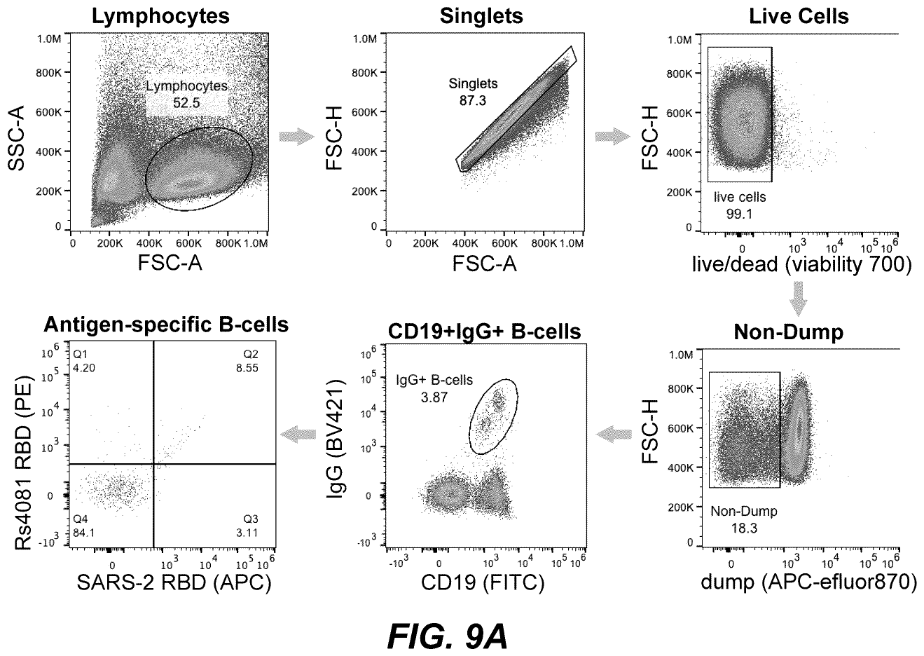

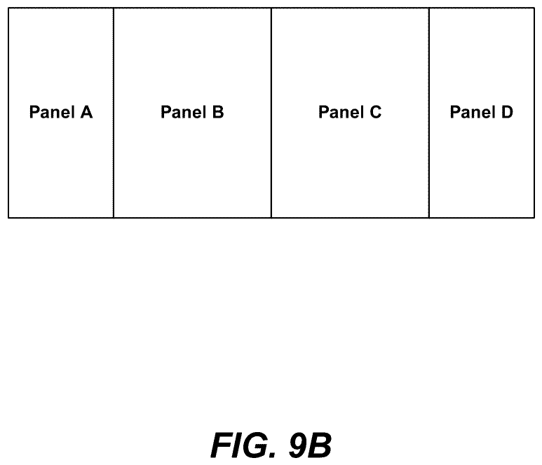

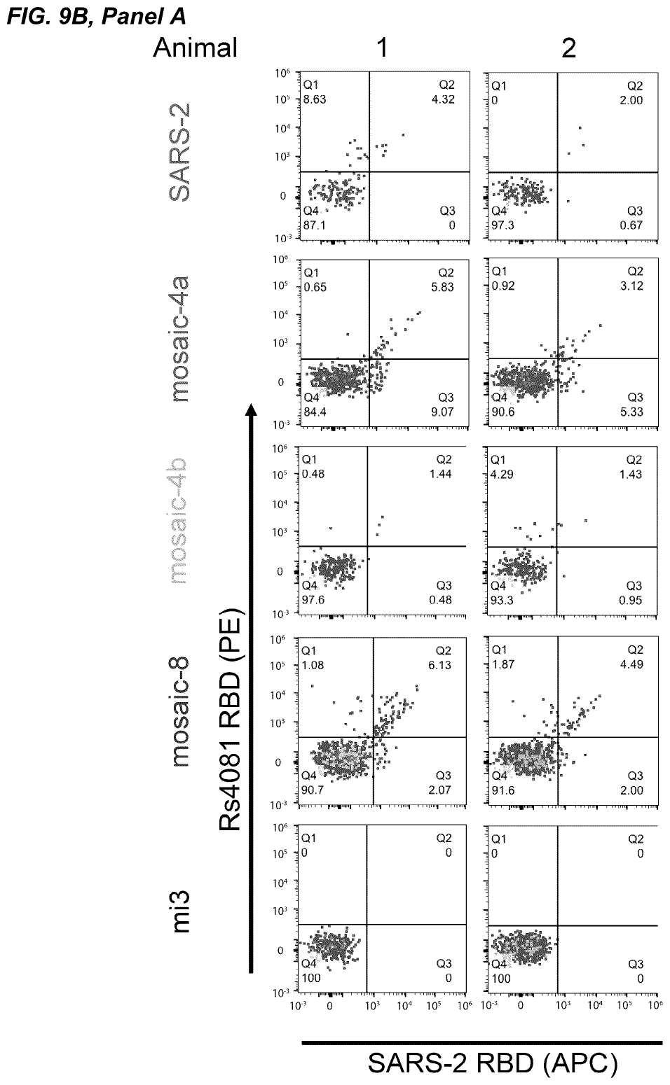

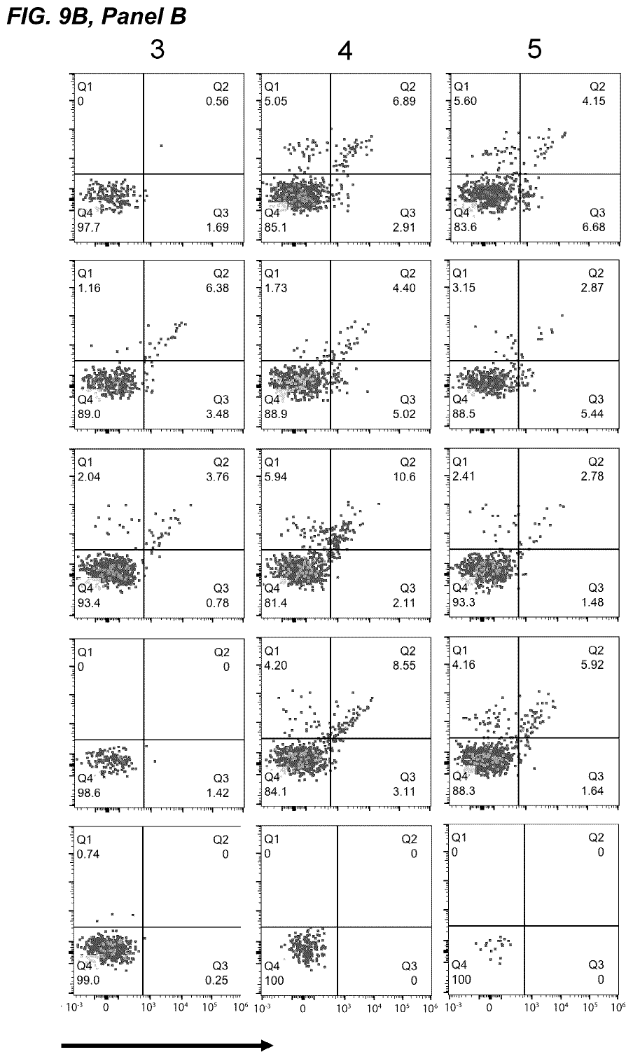

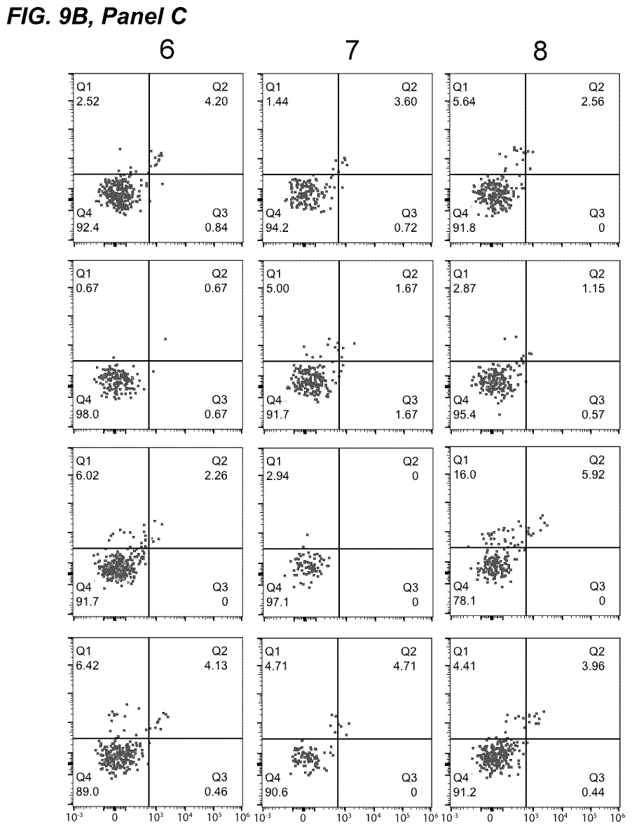

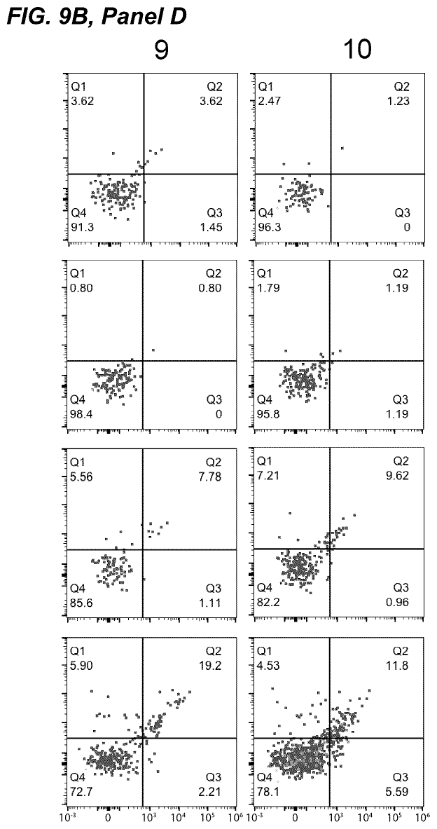

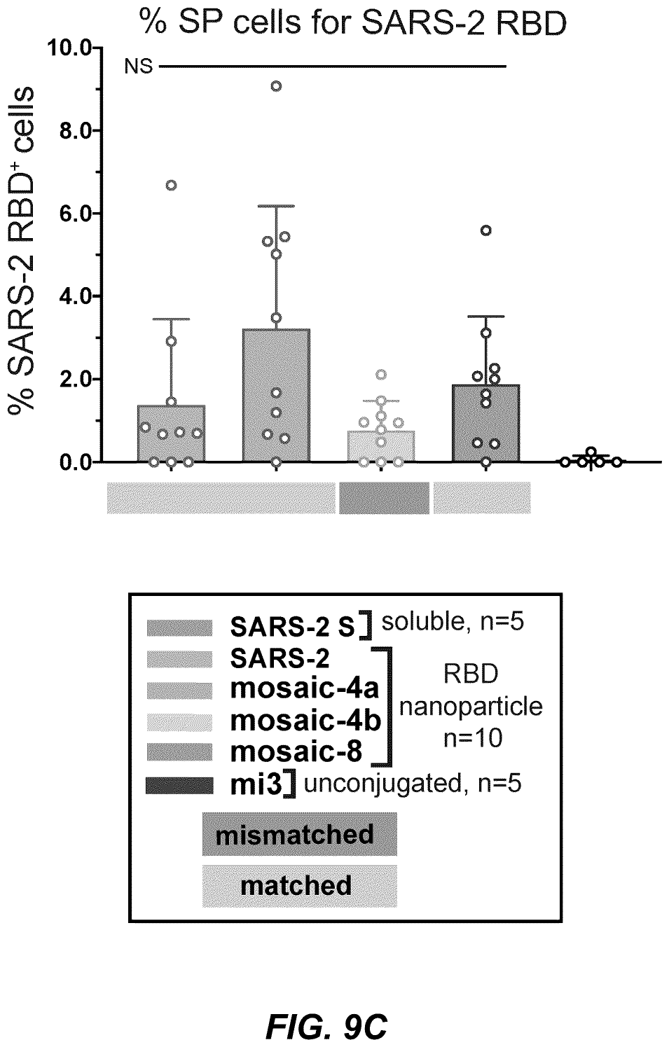

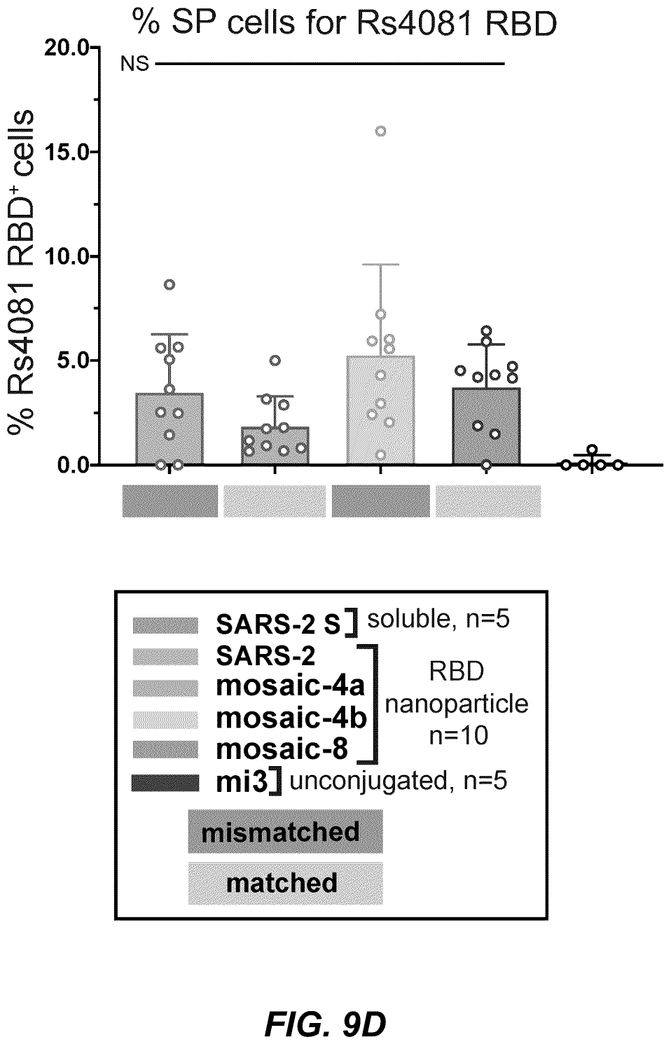

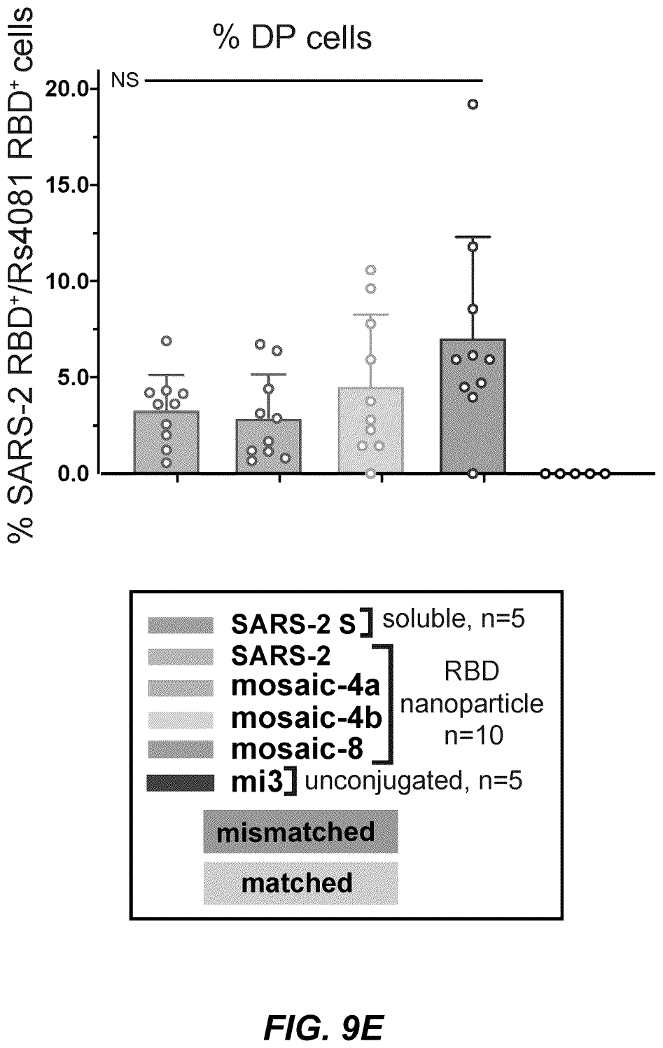

The potential for cross-reactive recognition was investigated using flow cytometry to ask whether B-cell receptors on IgG+ splenic B-cells from RBD-nanoparticle-boosted animals could simultaneously recognize RBDs from SARS-2 and Rs4081 (related by 70% sequence identity) (FIG. 1D; FIG. 9A-FIG. 9E). Whereas control animals were negative, all other groups showed B-cells that recognized SARS-2 and Rs4081 RBDs simultaneously, suggesting the existence of antibodies that cross-react with both RBDs (FIG. 9E).

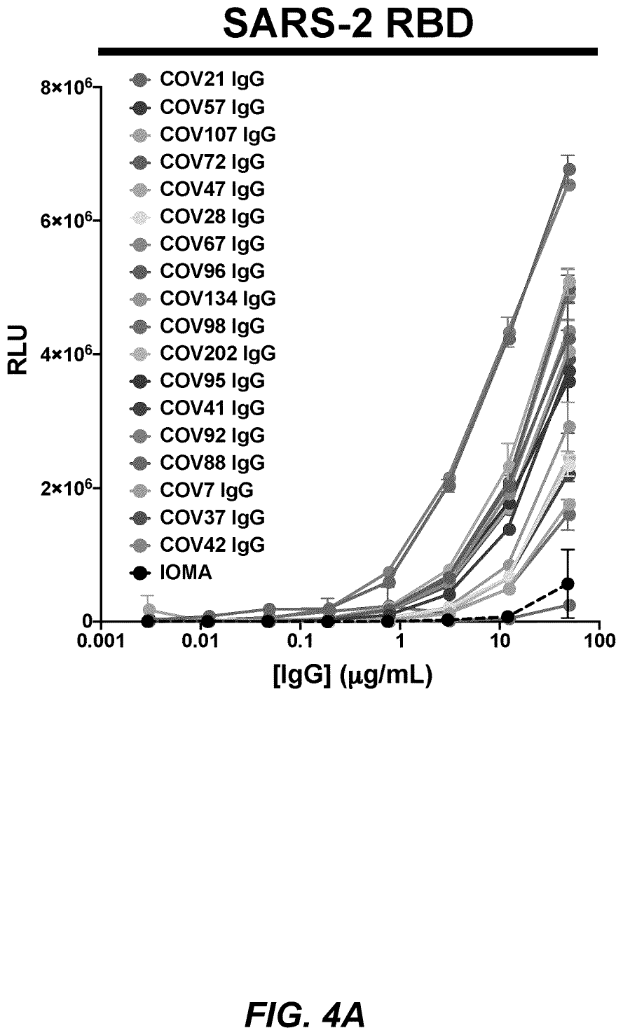

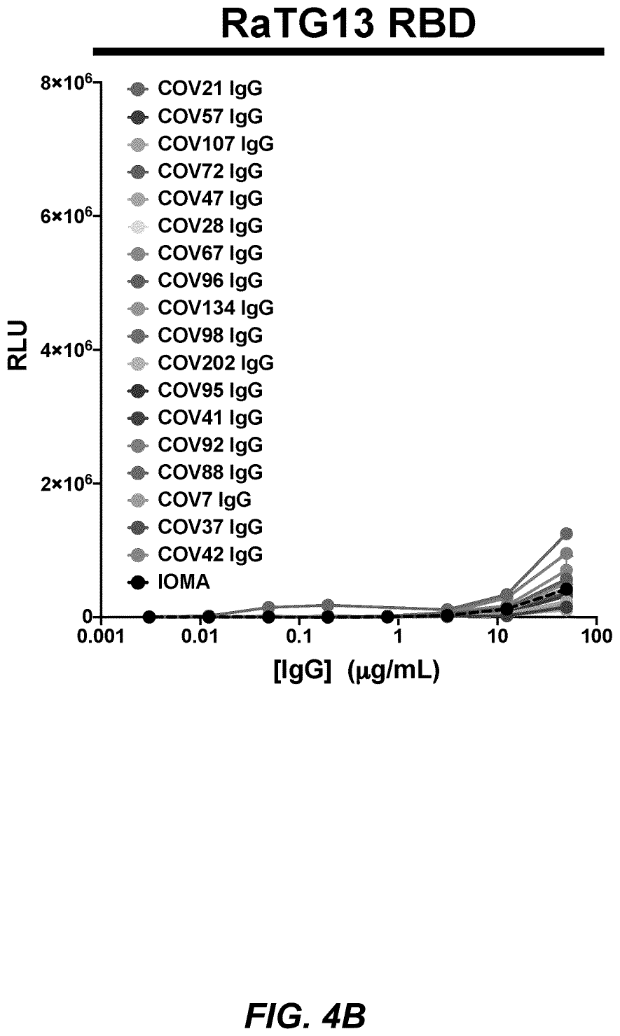

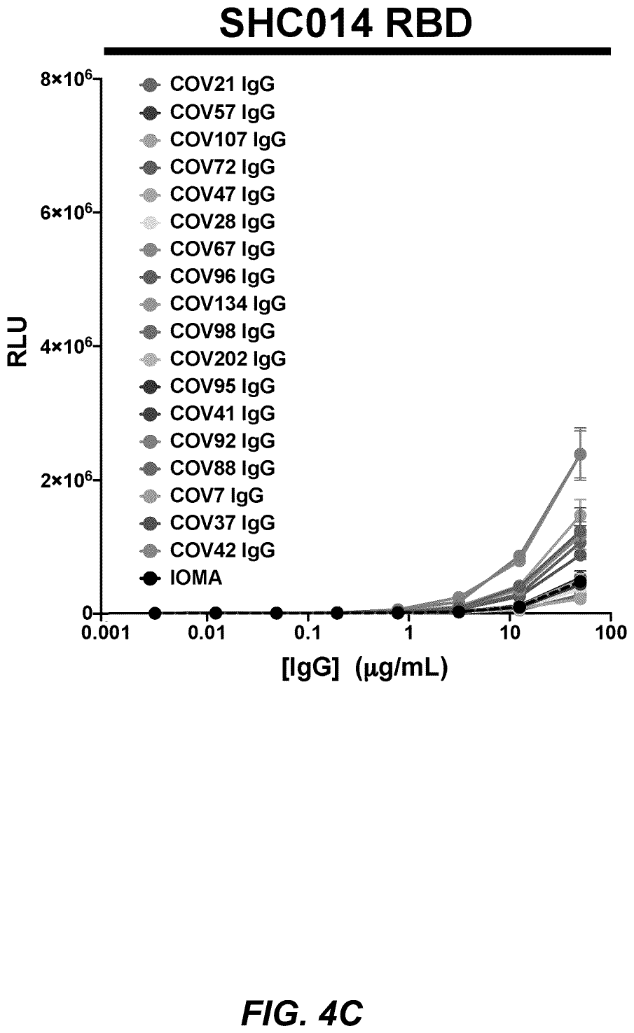

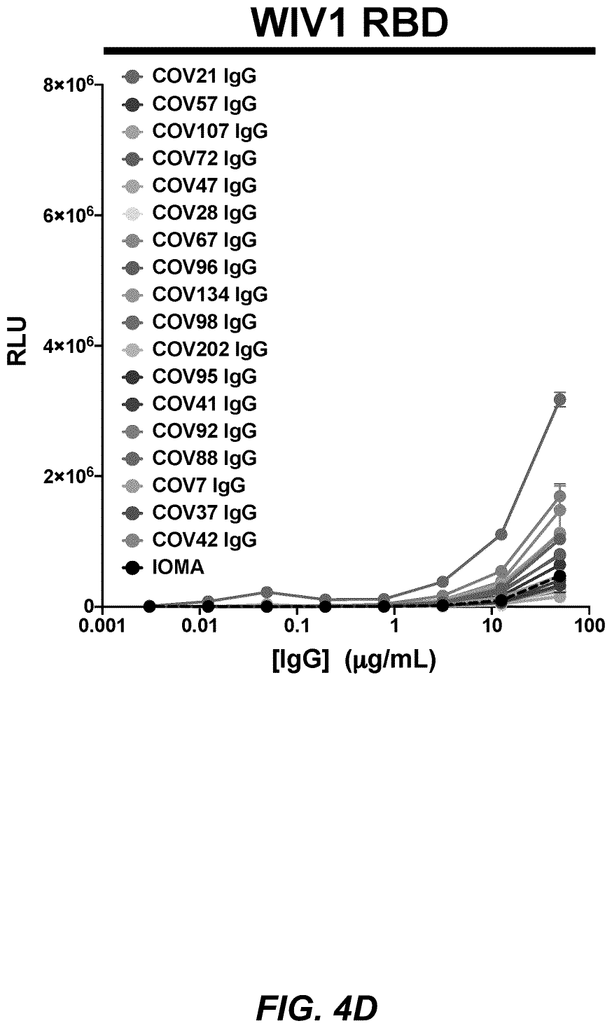

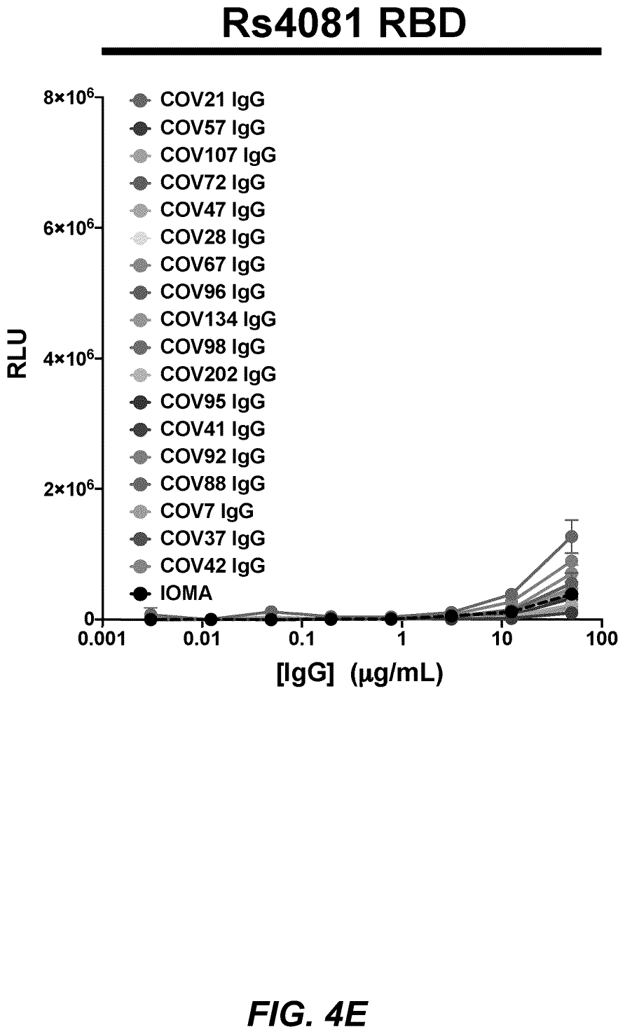

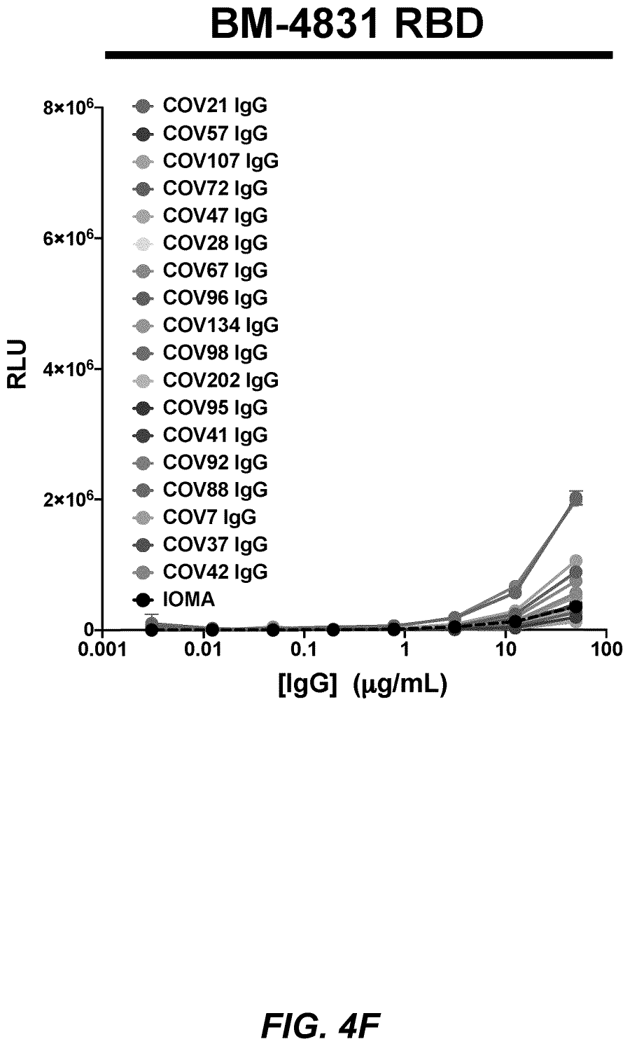

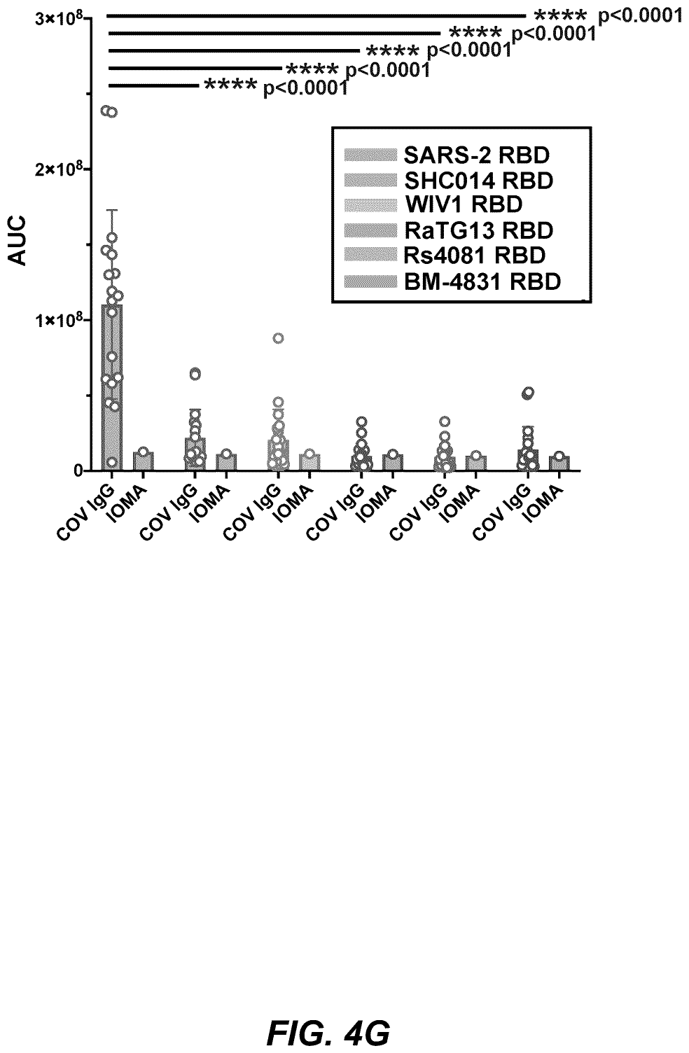

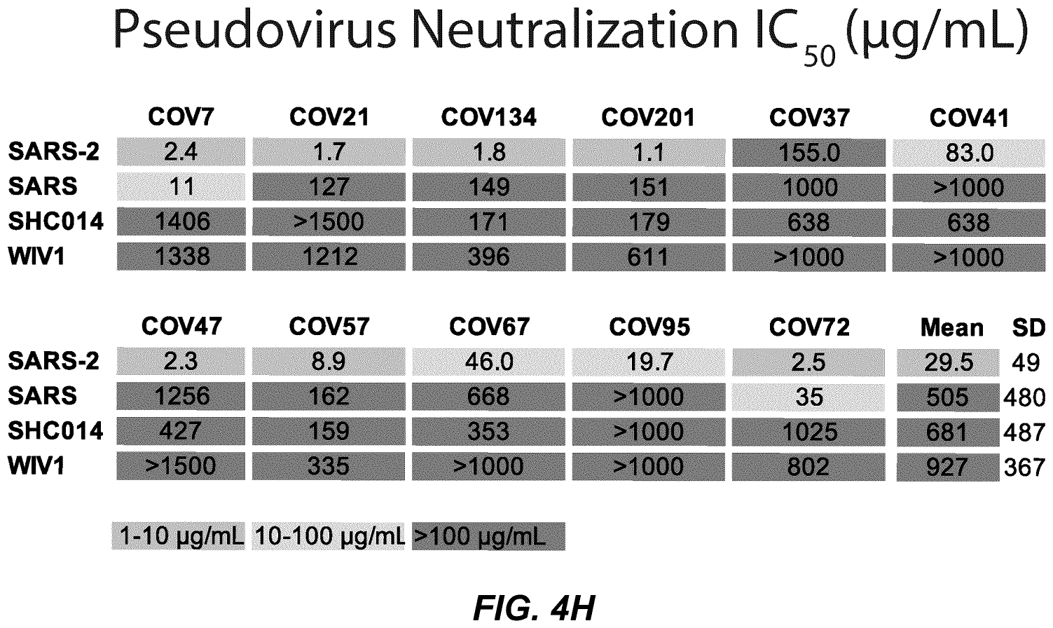

To compare antibodies elicited by RBD-nanoparticle immunization to antibodies elicited by SARS-CoV-2 infection, ELISAs against the RBD panel were repeated using IgGs from COVID-19 plasma donors (FIG. 4A-FIG. 4H). Most of the convalescent plasmas showed detectable binding to SARS-2 RBD (FIG. 4A). However, binding to other Sarbecovirus RBDs (RaTG13, SHC014, WIV1, Rs4081 and BM-4831) was significantly weaker than binding to SARS 2 RBD, with many human plasma IgGs showing no binding above background (FIG. 4B-FIG. 4G). In addition, although convalescent plasma IgGs neutralized SARS-CoV-2 pseudoviruses, they showed weak or no neutralization of SARS, SHC014, or WIV1 pseudoviruses (FIG. 4H). These results are consistent with little to no cross-reactive recognition of RBDs from zoonotic coronavirus strains resulting from SARS-CoV-2 infection in humans.

In conclusion, multimerization of RBDs on nanoparticles enhances immunogenicity compared with soluble antigen and homotypic SARS-2 nanoparticle immunization produced IgG responses that bound zoonotic RBDs and neutralized heterologous coronaviruses after boosting. By contrast, soluble SARS-2 S immunization and natural infection with SARS-CoV-2 resulted in weak or no heterologous responses in plasmas. Co-display of SARS-2 RBD along with diverse RBDs on mosaic nanoparticles showed no disadvantages for eliciting neutralizing antibodies against SARS-CoV-2 compared with homotypic SARS-2 nanoparticles, suggesting mosaic nanoparticles as a candidate vaccine to protect against COVID-19. Furthermore, compared with homotypic SARS-2 RBD particles, the mosaic co-display strategy demonstrated advantages for eliciting neutralizing antibodies against zoonotic Sarbecoviruses, thus potentially also providing protection against emerging coronaviruses with human spillover potential. Neutralization of matched and mismatched strains was observed after mosaic priming, suggesting a single injection of a mosaic-RBD nanoparticle might be sufficient in a vaccine. Since COVID-19 convalescent plasmas showed little to no recognition of coronavirus RBDs other than SARS-CoV-2, COVD-19-induced immunity in humans may not protect against another emergent coronavirus. Embodiments of the mosaic nanoparticles described herein could be used to present RBDs from newly-discovered zoonotic coronaviruses.

### Example 2

### Exemplary Materials and Methods

The following exemplary materials and methods are used in the preparation and practice of the embodiments disclosed herein.

Phylogenetic tree. A sequence alignment of coronavirus RBD domains was made using Clustal Omega. A phylogenetic tree was calculated from this amino acid alignment using PhyML 3.0, and a figure of this tree was made using PRESTO (http://www.atgc-montpellier.fr/presto).

Expression of RBD and S proteins. Mammalian expression vectors encoding the RBDs of SARS-CoV-2 (GenBank MN985325.1; S protein residues 319-537; SEQ ID NO: 1) and SARS-CoV S (GenBank AAP13441.1; residues 318-510; SEQ ID NO: 9) with an N-terminal human IL-2 or Mu phosphatase signal peptide were previously described. Expression vectors were constructed similarly for RBDs from the following other Sarbecovirus strains: RaTG13-CoV (GenBank QHR63300; S protein residues 319-541; SEQ ID NO: 2), SHC014-CoV (GenBank KC881005; residues 307-524; SEQ ID NO: 3), Rs4081-CoV (GenBank KY417143; S protein residues 310-513; SEQ ID NO: 4), pangolin17-CoV (GenBank QIA48632; residues 317-539; SEQ ID NO: 5), RmYN02-CoV (GSAID EPI_ISL_412977; residues 298-501; SEQ ID NO: 6), Rf1-CoV (GenBank DQ412042; residues 310-513; SEQ ID NO: 7), WIV1-CoV (GenBank KF367457; residues 307-528; SEQ ID NO: 8), Yun11-CoV (GenBank JX993988; residues 310-513; SEQ ID NO: 10), BM-4831-CoV (GenBank NC014470; residues 310-528; SEQ ID NO: 11), BtkY72-CoV (GenBank KY352407; residues 309-530; SEQ ID NO: 12). Two versions of each RBD expression vector were made: one including a C-terminal hexahistidine tag (G-HHHHHH, SEQ ID NO: 13) and SpyTag003 (RGVPHIVMVDAYKRYK, SEQ ID NO: 14) (for coupling to SpyCatcher003-mi3) and one with only a hexahistidine tag (for ELISAs). Biotinylated SARS-CoV-2 and Rs4081 RBDs were produced by co-transfection of Avi/His-tagged RBD expression plasmids with an expression plasmid encoding an ER-directed BirA enzyme (kind gift of Michael Anaya, Caltech). RBD proteins were purified from transiently-transfected Expi293F cell (Gibco) supernatants by nickel affinity and size-exclusion chromatography. Peak fractions corresponding to RBDs were identified by SDS-PAGE and then pooled and stored at 4° C. A trimeric SARS-CoV-2 ectodomain with 6P stabilizing mutations was expressed and purified as described. Correct folding of the soluble SARS-CoV-2 S trimer was verified by a 3.3 Å cryo-EM structure of a neutralizing antibody complexed with the trimer preparation used for immunizations. To prepare fluorochrome-conjugated streptavidin-tetramerized RBDs, biotinylated SARS-2 and Rs4081 RBDs were incubated with streptavidin-APC (eBioscience™) and streptavidin-PE (ThermoFisher), respectively, overnight at 4° C. at a 1:1 molar ratio of RBD to streptavidin subunit.

Preparation of human plasma IgGs. Plasma samples collected from COVID-19 convalescent and healthy donors are described in Robbiani et al., Nature 584, 437-442 (2020). Human IgGs were isolated from heat-inactivated plasma samples using 5-mL HiTrap MabSelect SuRe columns (GE Healthcare Life Sciences) as described.

Preparation of RBD-mi3 nanoparticles. SpyCatcher003-mi3 particles were prepared by purification from BL21 (DE3)-RIPL E coli (Agilent) transformed with a pET28a SpyCatcher003-mi3 gene (including an N-terminal 6x-His tag) as described in Cohen A A et al, 2021, PLOS ONE 16(3): e0247963. Briefly, cell pellets from transformed bacteria were lysed with a cell disruptor in the presence of 2.0 mM PMSF (Sigma). Lysates were spun at 21,000×g for 30 min, filtered with a 0.2 μm filter, and mi3 particles were isolated by Ni-NTA chromatography using a pre-packed HisTrap™ HP column (GE Healthcare). Eluted particles were concentrated using an Amicon Ultra 15 mL 30K concentrator (MilliporeSigma) and purified by SEC using a HiLoad® 16/600 Superdex® 200 (GE Healthcare) column equilibrated with 25 mM Tris-HCl pH 8.0, 150 mM NaCl, 0.02% NaN₃ (TBS). SpyCatcher-mi3 particles were stored at 4° C. and used for conjugations for up to 1 month after filtering with a 0.2 μm filter or spinning at 21,000×g for 10 min.

Purified SpyCatcher003-mi3 was incubated with a 3-fold molar excess (RBD to mi3 subunit) of purified SpyTagged RBD (either a single RBD for making homotypic SARS-CoV-2 RBD particles or an equimolar mixture of four or eight RBDs for making mosaic particles) overnight at room temperature in TBS. Conjugated mi3 particles were separated from free RBDs by SEC on a Superose 6 10/300 column (GE Healthcare) equilibrated with PBS (20 mM sodium phosphate pH 7.5, 150 mM NaCl). Fractions corresponding to conjugated mi3 particles were collected and analyzed by SDS-PAGE. Concentrations of conjugated mi3 particles were determined using a Bio-Rad Protein Assay.

Immunizations. Animal procedures and experiments were performed according to protocols approved by the IACUC. Experiments were done using 4-6 week old female Balb/c mice (Charles River Laboratories), with 5 animals each for cohorts immunized with soluble SARS-CoV-2 S or SpyCatcher003-mi3, and 10 animals each for remaining cohorts (FIG. 3A). Immunizations were carried out with intraperitoneal (ip) injections of either 5 μg of conjugated RBD (calculated as the mass of the RBD, assuming 100% efficiency of conjugation to SpyCatcher003-mi3), 5 μg of soluble SARS-CoV-2 S, or 6 μg of unconjugated SpyCatcher003-mi3, in 100 μL of 50% v/v AddaVax™ adjuvant (Invivogen). Animals were boosted 4 weeks after the prime with the same quantity of antigen in adjuvant. Animals were bled every 2 weeks via tail veins, and then euthanized 8 weeks after the prime (Day 56, 57) and bled through cardiac puncture. Blood samples were allowed to clot at room temperature in MiniCollect® Serum and Plasma Tubes (Greiner), and serum was harvested, preserved in liquid nitrogen, and stored at −80° C. until use.

Sera for ELISAs were collected at Day 14 (Prime) and Day 42 (Boost). Sera for neutralization assays were collected at Day 28 (Prime) and Day 56 (Boost) (FIG. 3A-FIG. 3F, FIG. 7A-FIG. 7F).

ELISAs. 10 μg/ml of a purified RBD (not SpyTagged) in 0.1 M NaHCO₃ pH 9.8 was coated onto Nunc® MaxiSorp™ 384-well plates (Sigma) and stored overnight at 4° C. Plates were washed with Tris-buffered saline with 0.1% Tween 20 (TBS-T) after blocking with 3% bovine serum albumin (BSA) in TBS-T for 1 hr at room temperature. Mouse serum was diluted 1:100 and then serially diluted by 4-fold with TBS-T/3% BSA and added to plates for 3 hr at room temperature. A 1:50,000 dilution of secondary HRP-conjugated goat anti-mouse IgG (Abcam) was added after washing for 1 hr at room temperature. Plates were developed using SuperSignal™ ELISA Femto Maximum Sensitivity Substrate (ThermoFisher) and read at 425 nm. Curves were plotted and integrated to obtain the area under the curve (AUC) using Graphpad Prism 8.3 assuming a one-site binding model with a Hill coefficient (FIG. 3A-FIG. 3F, FIG. 7A-FIG. 7F). EC₅₀s and endpoint titers were also calculated, which were determined using the dilution that was at or below the mean+2×the standard deviation of the plate control (no primary serum added) for ELISA binding data (FIG. 7E-FIG. 7F). AUC calculations were used as they better capture changes in maximum binding. Statistical significance of titer differences between groups were calculated using Tukey's multiple comparison test using Graphpad Prism 8.3.

Neutralization assays. SARS-CoV-2, SARS, WIV1, and SHC014 pseudoviruses based on HIV lentiviral particles were prepared as described using genes encoding S protein sequences lacking C-terminal residues in the cytoplasmic tail: 21 amino acid deletions for SARS-CoV-2, WIV1, and SHC014 and a 19 amino acid deletion for SARS-CoV. IC₅₀ values derived from this pseudotyped neutralization assay method were shown to quantitatively correlate with results from neutralization assays using authentic SARS-CoV-2 virus. For pseudovirus neutralization assays, four-fold serially diluted sera from immunized mice were incubated with a pseudotyped virus for 1 hour at 37° C. After incubation with 293T_{ACE2} target cells for 48 hours at 37° C., cells were washed twice with phosphate-buffered saline (PBS) and lysed with Luciferase Cell Culture Lysis 5× reagent (Promega). NanoLuc Luciferase activity in lysates was measured using the Nano-Glo Luciferase Assay System (Promega). Relative luminescence units (RLUs) were normalized to values derived from cells infected with pseudotyped virus in the absence of serum. Half-maximal inhibitory dilutions (ID₅₀ values) were determined using 4-parameter nonlinear regression in AntibodyDatabase. Statistical significance of titer differences between groups were calculated using Tukey's multiple comparison test of ID₅₀s converted to log¹⁰ scale using Graphpad Prism 8.3.

Statistical Analysis. Comparisons between groups for ELISAs and neutralization assays were calculated with one-way analysis of variance (ANOVA) using Tukey's post hoc test in Prism 9.0 (Graphpad). For correlation analysis between ELISA and neutralization titers, significance (p), Spearman coefficients (rₛ), and linear plots were calculated using Prism 9.0 (Graphpad). Differences were considered significant when p values were less than 0.05. Exact p values are in relevant drawings near each corresponding line, with asterisks denoting level of significance (* denotes 0.01<p<0.05, ** denotes 0.001<p<0.01, *** denotes 0.0001<p<0.001, and **** denotes p<0.0001).

Flow cytometry. B-cell analysis using flow cytometry was carried out as described. Briefly, single-cell suspensions were prepared from mouse spleens using mechanical dissociation, and red blood cells were removed using ACK lysing buffer (Gibco). The white blood cell preparation was enriched for IgG+ B-cells using the negative selection protocol in a mouse memory B-cell isolation kit (Miltenyi). The following commercial reagents were used to stain enriched splenocytes: CD4-APC-eFluor 780 (clone: RM4-5), F4/80-APC-eFluor 780 (clone: BM8), CD8a-APC-eFluor 780 (clone: 53-6.7), Ly-6G-APC-eFluor 780 (clone: RB6-8C5), IgM-APC-eFluor 780 (clone: II/41) (Thermo Fisher Scientific), CD19-FITC (clone: 6D5) (Biolegend), IgG1 BV421 (clone: X40) and IgG2 BV421 (clone: R19-15) (BD Bioscience). SARS-2 RBD-APC and Rs4081 RBD-PE for used to identify antigen-specific B-cells. Cell viability was analyzed with Fixable Viability Stain 700 (BD Bioscience). Stained cells were analyzed with a SY3200 Cell Sorter (Sony) configured to detect 6 fluorochromes. 2,000,000 events were collected per sample and analyzed via FlowJo software (TreeStar).

### Terminology

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of obtaining a B cell producing a cross-reactive antibody, comprising:
immunizing a mammal with a vaccine composition comprising a multivalent carrier displaying a first plurality of antigens; and
isolating one or more B-cells from said mammal capable of binding a second plurality of antigens, thereby obtaining a b cell producing a cross-reactive antibody.

2. The method of claim 1, wherein at least one antigen of the first plurality of antigens and the second plurality of antigens is shared.

3. The method of claim 1, wherein at least one antigen of the first plurality of antigens and the second plurality of antigens is different.

4. The method of claim 1, wherein the second plurality of antigens comprises a subset of the first plurality of antigens.

5. (canceled)

6. The method of claim 1, wherein the first plurality of antigens and/or the second plurality of antigens are viral polypeptides.

7. The method of claim 1, wherein the first plurality of antigens and/or the second plurality of antigens comprise viral polypeptides of a same protein type.

8. The method of claim 1, wherein the first plurality of antigens and/or the second plurality of antigens comprise viral polypeptides of a different protein types.

9. The method of claim 1, wherein the first plurality of antigens and/or the second plurality of antigens comprises a first viral polypeptide of a first virus and a second viral polypeptide of a second virus that is different from the first virus.

10.-13. (canceled)

14. The method of claim 1, wherein the multivalent carrier comprises a nanoparticle selected from the group consisting of: lipid-based nanoparticles, polymeric nanoparticles, inorganic nanoparticles, surfactant-based emulsions, nanowires, silica nanoparticles, virus-like particles, peptide or protein-based particles, lipid-polymer particles, nanolipoprotein particles, and combinations thereof.

15.-26. (canceled)

27. The method of claim 1, wherein the method comprises administering the vaccine composition to the mammal two or more times.

28. The method of claim 1, wherein the isolating step is performed about 1 day, 2 days, 4 days, 1 week, 10 days, 2 weeks, 3 weeks, 4weeks, 5 weeks, 6 weeks, 7 weeks, 8 weeks, 9 weeks, or 10 weeks, after the immunizing step.

29.-31. (canceled)

32. The method of claim 1, wherein the isolating step comprises positive selection of B cells and/or negative selection of non-B-cells.

33.-34. (canceled)

35. The method of claim 1, wherein the isolating step comprises isolating splenocytes.

36. The method of claim 1, wherein the cross-reactive antibody is a cross-neutralizing antibody.

37.-59. (canceled)

60. The method of claim 1, wherein the plurality of coronavirus antigens are of coronaviruses in the subgenus of Sarbecovirus.

61.-64. (canceled)

65. An antibody or fragment thereof derived from a B-cell obtained by the method of claim 1.

66. A nucleic acid molecule encoding the antibody or fragment thereof of claim 65.

67. An antibody or fragment thereof derived from recombinant expression of the nucleic acid molecule of claim 66.

68. (canceled)

69. A pharmaceutical composition comprising the antibody or fragment thereof of claim 65.

70. A method of treating or preventing a disease or disorder caused by a viral infection in a subject in need thereof, comprising: administering to the subject a pharmaceutically effective amount of the pharmaceutical composition of claim 69, thereby treating or preventing the disease or disorder caused by the viral infection in the subject.

71.-81. (canceled)


SEQUENCE LISTING (26 sequences)

SEQ ID NO: 1 - protein, 219 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNK

SEQ ID NO: 2 - protein, 223 aa, synthetic construct
RVQPTDSIVRFPNITNLCPFGEVFNATTFASVYAWNRKRISNCVADYSVLYNSTSFSTFKCYGVSPTKLNDLCFTNVYADSFVITGDEVRQIAPGQTGKIADYNYKLPDDFTGCVIAWNSKHIDAKEGGNFNYLYRLFRKANLKPFERDISTEIYQAGSKPCNGQTGLNCYYPLYRYGFYPTDGVGHQPYRVVVLSFELLNAPATVCGPKKSTNLVKNKCVNF

SEQ ID NO: 3 - protein, 218 aa, synthetic construct
RVAPSKEVVRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFLGCVLAWNTNSKDSSTSGNYNYLYRWVRRSKLNPYERDLSNDIYSPGGQSCSAVGPNCYNPLRPYGFFTTAGVGHQPYRVVVLSFELLNAPATVCGPKLSTDLIKNQ

SEQ ID NO: 4 - protein, 204 aa, synthetic construct
RVSPTHEVVRFPNITNRCPFDKVFNASRFPNVYAWERTKISDCVADYTVLYNSTSFSTFKCYGVSPSKLIDLCFTSVYADTFLIRSSEVRQVAPGETGVIADYNYKLPDDFTGCVIAWNTAKQDQGQYYYRSSRKTKLKPFERDLTSDENGVRTLSTYDFYPNVPIEYQATRVVVLSFELLNAPATVCGPKLSTALVKNQCVNF

SEQ ID NO: 5 - protein, 223 aa, synthetic construct
RVQPTISIVRFPNITNLCPFGEVFNASKFASVYAWNRKRISNCVADYSVLYNSTSFSTFKCYGVSPTKLNDLCFTNVYADSFVVKGDEVRQIAPGQTGVIADYNYKLPDDFTGCVIAWNSVKQDALTGGNYGYLYRLFRKSKLKPFERDISTEIYQAGSTPCNGQVGLNCYYPLERYGFHPTTGVNYQPFRVVVLSFELLNGPATVCGPKLSTTLVKDKCVNF

SEQ ID NO: 6 - protein, 204 aa, synthetic construct
RILPSTEVVRFPNITNFCPFDKVFNATRFPNVYAWQRTKISDCIADYTVLYNSTSFSTFKCYGVSPSKLIDLCFTSVYADTFLIRFSEVRQIAPGETGVIADYNYKLPDDFTGCVLAWNTAQQDIGSYFYRSHRAVKLKPFERDLSSDENGVRTLSTYDFNPNVPLDYQATRVVVLSFELLNAPATVCGPKLSTQLVKNRCVNF

SEQ ID NO: 7 - protein, 204 aa, synthetic construct
RVSPVTEVVRFPNITNLCPFDKVFNATRFPSVYAWERTKISDCVADYTVFYNSTSFSTFNCYGVSPSKLIDLCFTSVYADTFLIRFSEVRQVAPGQTGVIADYNYKLPDDFTGCVIAWNTAKQDVGSYFYRSHRSSKLKPFERDLSSEENGVRTLSTYDFNQNVPLEYQATRVVVLSFELLNAPATVCGPKLSTSLVKNQCVNF

SEQ ID NO: 8 - protein, 222 aa, synthetic construct
RVAPSKEVVRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFTGCVLAWNTRNIDATQTGNYNYKYRSLRHGKLRPFERDISNVPFSPDGKPCTPPAFNCYWPLNDYGFYITNGIGYQPYRVVVLSFELLNAPATVCGPKLSTDLIKNQCVNF

SEQ ID NO: 9 - protein, 193 aa, synthetic construct
NITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATV

SEQ ID NO: 10 - protein, 204 aa, synthetic construct
RVSPSTEVIRFPNITNRCPFDRVFNASRFPSVYAWERTKISDCVADYTVLYNSTSFSTFKCYGVSPSKLIDLCFTSVYADTFLIRFSEVRQIAPGETGVIADYNYKLPDEFTGCVIAWNTANQDRGQYYYRSSRKTKLKPFERDLSSDENGVRTLSTYDFYPSVPLEYQATRVVVLSFELLNAPATVCGPKLSTSLIKNQCVNF

SEQ ID NO: 11 - protein, 219 aa, synthetic construct
RVTPTTEVVRFPNITQLCPFNEVFNITSFPSVYAWERMRITNCVADYSVLYNSSASFSTFQCYGVSPTKLNDLCFSSVYADYFVVKGDDVRQIAPAQTGVIADYNYKLPDDFTGCVIAWNTNSLDSSNEFFYRRFRHGKIKPYGRDLSNVLFNPSGGTCSAEGLNCYKPLASYGFTQSSGIGFQPYRVVVLSFELLNAPATVCGPKQSTELVKNKCVNF

SEQ ID NO: 12 - protein, 222 aa, synthetic construct
RVSPSTEVVRFPNITNLCPFGQVFNASNFPSVYAWERLRISDCVADYAVLYNSSSSFSTFKCYGVSPTKLNDLCFSSVYADYFVVKGDDVRQIAPAQTGVIADYNYKLPDDFTGCVLAWNTNSVDSKSGNNFYYRLFRHGKIKPYERDISNVLYNSAGGTCSSISQLGCYEPLKSYGFTPTVGVGYQPYRVVVLSFELLNAPATVCGPKKSTELVKNKCVNF

SEQ ID NO: 13 - protein, 7 aa, synthetic construct
GHHHHHH

SEQ ID NO: 14 - protein, 16 aa, synthetic construct
RGVPHIVMVDAYKRYK

SEQ ID NO: 15 - protein, 223 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 16 - protein, 223 aa, synthetic construct; identical to SEQ ID NO: 2

SEQ ID NO: 17 - protein, 222 aa, synthetic construct
RVAPSKEVVRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFLGCVLAWNTNSKDSSTSGNYNYLYRWVRRSKLNPYERDLSNDIYSPGGQSCSAVGPNCYNPLRPYGFFTTAGVGHQPYRVVVLSFELLNAPATVCGPKLSTDLIKNQCVNF

SEQ ID NO: 18 - protein, 206 aa, synthetic construct
RVSPTHEVVRFPNITNRCPFDKVFNASRFPNVYAWERTKISDCVADYTVLYNSTSFSTFKCYGVSPSKLIDLCFTSVYADTFLIRSSEVRQVAPGETGVIADYNYKLPDDFTGCVIAWNTAKQDQGQYYYRSSRKTKLKPFERDLTSDENGVRTLSTYDFYPNVPIEYQATRVVVLSFELLNAPATVCGPKLSTALVKNQCVNFNF

SEQ ID NO: 19 - protein, 223 aa, synthetic construct; identical to SEQ ID NO: 5

SEQ ID NO: 20 - protein, 206 aa, synthetic construct
RILPSTEVVRFPNITNFCPFDKVFNATRFPNVYAWQRTKISDCIADYTVLYNSTSFSTFKCYGVSPSKLIDLCFTSVYADTFLIRFSEVRQIAPGETGVIADYNYKLPDDFTGCVLAWNTAQQDIGSYFYRSHRAVKLKPFERDLSSDENGVRTLSTYDFNPNVPLDYQATRVVVLSFELLNAPATVCGPKLSTQLVKNRCVNFNF

SEQ ID NO: 21 - protein, 206 aa, synthetic construct
RVSPVTEVVRFPNITNLCPFDKVFNATRFPSVYAWERTKISDCVADYTVFYNSTSFSTFNCYGVSPSKLIDLCFTSVYADTFLIRFSEVRQVAPGQTGVIADYNYKLPDDFTGCVIAWNTAKQDVGSYFYRSHRSSKLKPFERDLSSEENGVRTLSTYDFNQNVPLEYQATRVVVLSFELLNAPATVCGPKLSTSLVKNQCVNFNF

SEQ ID NO: 22 - protein, 222 aa, synthetic construct; identical to SEQ ID NO: 8

SEQ ID NO: 23 - protein, 222 aa, synthetic construct
RVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNF

SEQ ID NO: 24 - protein, 206 aa, synthetic construct
RVSPSTEVIRFPNITNRCPFDRVFNASRFPSVYAWERTKISDCVADYTVLYNSTSFSTFKCYGVSPSKLIDLCFTSVYADTFLIRFSEVRQIAPGETGVIADYNYKLPDEFTGCVIAWNTANQDRGQYYYRSSRKTKLKPFERDLSSDENGVRTLSTYDFYPSVPLEYQATRVVVLSFELLNAPATVCGPKLSTSLIKNQCVNFNF

SEQ ID NO: 25 - protein, 221 aa, synthetic construct
RVTPTTEVVRFPNITQLCPFNEVFNITSFPSVYAWERMRITNCVADYSVLYNSSASFSTFQCYGVSPTKLNDLCFSSVYADYFVVKGDDVRQIAPAQTGVIADYNYKLPDDFTGCVIAWNTNSLDSSNEFFYRRFRHGKIKPYGRDLSNVLFNPSGGTCSAEGLNCYKPLASYGFTQSSGIGFQPYRVVVLSFELLNAPATVCGPKQSTELVKNKCVNFNF

SEQ ID NO: 26 - protein, 222 aa, synthetic construct; identical to SEQ ID NO: 12